(19) United States
(12) Patent Application Publication
(10) Pub. No.: US 2026/0311821 A1
(43) Pub. Date: Oct. 8, 2026

(54) SYSTEMS AND METHODS FOR AI-BASED IONIZABLE LIPID DEVELOPMENT FOR LIPID NANOPARTICLE (LNP)-BASED CARGO MOLECULE DELIVERY

(71) Applicant: The Governing Council of The University of Toronto, Toronto (CA)

(72) Inventors: Bowen Li, Vaughan (CA); Bo Wang, Maple (CA); Yue Xu, Toronto (CA); Shihao Ma, Toronto (CA); Haotian Cui, North York (CA); Jingan Chen, Toronto (CA)

(21) Appl. No.: 19/484,151

(22) PCT Filed: May 13, 2024
(86) PCT No.: PCT/CA2024/050644

§ 371 Date: Nov. 13, 2025

Related U.S. Application Data
(60) Provisional application No. 63/502,091, filed on May 13, 2023.

(51) Int. Cl. G16C 20/70; G06N 20/00; G16C 60/00

(52) U.S. Cl. CPC G16C 20/70; G06N 20/00; G16C 60/00

(57) ABSTRACT

Various embodiments for AI-based systems and methods for engineering ionizable lipids for lipid nanoparticle (LNP)-based cargo molecule delivery are disclosed herein. In one example embodiment, the method comprises: designing a plurality of ionizable lipid structures based on a combination of a plurality of lipid headgroups, a plurality of first tailgroups and a plurality of second tailgroups; training a machine learning model based on the plurality of ionizable lipid structures; subsequently, selecting a first subset of ionizable lipid structures from the plurality of ionizable lipid structures and designating the first subset of ionizable lipid structures as experimental ionizable lipid structures; synthesizing, for example using a high throughput synthetic method, each of the experimental ionizable lipid structures; preparing a first set of separate LNPs, each LNP comprising a unique experimental ionizable lipid structure and the cargo molecule; determining a corresponding cargo molecule transfection efficiency value of each of the prepared LNPs in a target cell; further training the machine learning model based on the prepared LNPs and the corresponding cargo molecule transfection efficiency values; and deploying the further trained machine learning model to identify at least one lead ionizable lipid structure.

### FIELD

Various embodiments are described herein that generally relate to systems and methods for AI-based development of ionizable lipids for use in nanoparticles (LNPs) for example for messenger RNA (mRNA) delivery.

### BACKGROUND

The following paragraphs are provided by way of background to the present disclosure. They are not, however, an admission that anything discussed therein is prior art or part of the knowledge of persons skilled in the art.

Ionizable lipid nanoparticles pivotal to the success of COVID-19 mRNA vaccines hold substantial promise for expanding the landscape of therapies based on LNP delivery. Nevertheless, the risk of delivery to off-target tissues highlight the need for LNPs with enhanced tissue selectivity. The intricate nature of biological systems and inadequate knowledge of lipid structure-activity relationships emphasize the significance of high-throughput methods to produce chemically diverse lipid libraries for LNP delivery screening.

There is a need for a system and method that addresses the challenges and/or shortcomings described above.

### SUMMARY OF VARIOUS EMBODIMENTS

Various embodiments of a system and method for are provided according to the teachings herein.

According to one aspect of the invention, there is disclosed an AI-based method for ionizable lipid engineering for lipid nanoparticle (LNP)-based cargo molecule delivery. The method comprises designing a plurality of ionizable lipid structures based on a combination of a plurality of lipid headgroups, a plurality of first tailgroups and a plurality of second tailgroups; training a machine learning model based on the plurality of ionizable lipid structures; subsequently, selecting a first subset of ionizable lipid structures from the plurality of ionizable lipid structures and designating the first subset of ionizable lipid structures as experimental ionizable lipid structures; synthesizing, for example using a high throughput synthetic method, each of the experimental ionizable lipid structures; preparing a first set of separate lipid nanoparticles (LNPs), each LNP comprising a unique experimental ionizable lipid structure and the cargo molecule; determining a corresponding cargo molecule transfection efficiency value of each of the prepared LNPs in a target cell; further training the machine learning model based on the prepared LNPs and the corresponding cargo molecule transfection efficiency values; and deploying the further trained machine learning model to identify at least one lead ionizable lipid structure.

In various embodiments, deploying the further trained machine learning model comprises selecting a second subset of ionizable lipid structures from the plurality of ionizable lipid structures and designating the second subset of ionizable lipid structures as ionizable lipid candidate structures; predicting the cargo molecule transfection efficiency values of each of the ionizable lipid candidate structures based on the further trained machine learning model; ranking the ionizable lipid candidate structures based on the corresponding predicted cargo molecule transfection efficiency values in accordance with a ranking scheme; and identifying the at least one lead ionizable lipid structure based on the ranking of the ionizable lipid candidate structures for in-vitro and/or in-vivo validation.

In various embodiments, the method further comprises ranking the ionizable lipid candidate structures by first ranking the corresponding cargo molecule transfection efficiency values based on identity of lipid headgroups; and subsequently, ranking the cargo molecule transfection efficiency values within each headgroup based on tailgroups.

In various embodiments, synthesizing the experimental ionizable lipid structures comprises utilizing a one pot method for synthesizing an ionizable lipid of Formula I:

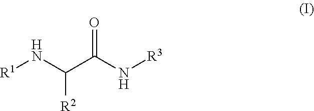

comprising reacting a compound of Formula A, a compound of Formula B and a compound of Formula C:

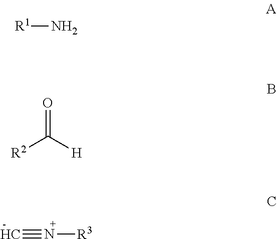

in the presence of a catalyst under conditions to provide the compound of Formula I,
wherein:
R¹ is a cyclic or acyclic group comprising at least one ionizable nitrogen atom;
R² is selected from C₁₋₃₀alkyl and C₂₋₃₀alkenyl, each of which is optionally interrupted by a hydrolysable group; and
R³ is selected from C₁₋₃₀alkyl and C₂₋₃₀alkenyl, each of which is optionally interrupted by a hydrolyzable group.

In at least one embodiment, the method of preparing the ionizable lipid of the above-noted Formula I is performed using combinatorial chemistry procedures.

In various embodiments, the method comprises generating the ionizable lipid candidate structures from the plurality of ionizable lipid structures by identifying ionizable lipid structures comprising cationic lipids.

In various embodiments, the method comprises generating the ionizable lipid candidate structures from the plurality of ionizable lipid structures by identifying ionizable lipid structures comprising ionizable lipids with alkyl chains that are either below a first length threshold or above a second length threshold.

In various embodiments, the method comprises generating the ionizable lipid candidate structures from the plurality of ionizable lipid structures by identifying ionizable lipid structures comprising a predetermined group of reagents.

In various embodiments, the cargo molecule comprises siRNA, tRNA, circRNA or mRNA, or a combination thereof.

In various embodiments, the target cell is selected from: Hela cells, T-cells, stem cells, lung epithelial cells, pancreatic beta cells, natural killer cells, macrophages, dendritic cells, endothelial cells, chondrocytes, skeletal muscle cells, cardiomyocytes, neurons, retinal cells and hepatocytes.

According to another aspect of the invention, there is disclosed an AI-based system for engineering ionizable lipids for lipid nanoparticle (LNP)-based cargo molecule delivery, where the system comprises a processor, a machine learning model coupled to the processor, and a memory coupled to the processor and the machine learning model, the memory comprising a database. The process is configured to design a plurality of ionizable lipid structures based on combinatorial of a plurality of lipid headgroups, a plurality of first tailgroup and a plurality of second tailgroup; store the plurality of ionizable lipid structures in the database in the memory; train the machine learning model based on the plurality of ionizable lipid structures; subsequently, select a first subset of ionizable lipid structures from the plurality of ionizable lipid structures and designate the first subset of ionizable lipid structures as experimental ionizable lipid structures; store the first subset of ionizable lipid structures in the database of the memory; receive a plurality of cargo molecule transfection efficiency values for a corresponding plurality of prepared lipid nanoparticles (LNPs), each LNP comprising a unique synthesized experimental ionizable lipid structure and the cargo molecule, wherein each experimental ionizable lipid structure is synthesized using a high throughput synthetic method and the cargo molecule transfection efficiency values are determined by testing the corresponding prepared lipid nanoparticle in a target cell; store the received plurality of cargo molecule transfection efficiency values in the database of the memory; further train the machine learning model based on the prepared LNPs and the corresponding cargo molecule transfection efficiency values; and deploy the further trained machine learning model to identify at least one lead ionizable lipid structure.

In various embodiments, the processor is further configured to select a second subset of ionizable lipid structures from the plurality of ionizable lipid structures and designate the second subset of ionizable lipid structures as ionizable lipid candidate structures, predict the cargo molecule transfection efficiency values of each of the ionizable lipid candidate structures based on the further trained machine learning model, rank the ionizable lipid candidate structures based on the corresponding predicted cargo molecule transfection efficiency values in accordance with a ranking schema, and identify the at least one lead ionizable lipid structure based on the ranking of the ionizable lipid candidate structures for in-vitro and/or in-vivo validation.

In various embodiments, the processor is further configured to rank the ionizable lipid candidate structures by first ranking the corresponding cargo molecule transfection efficiency values based on identity of lipid headgroups, and subsequently, rank the cargo molecule transfection efficiency values within each headgroup based on tailgroups.

In various embodiments, the processor is further configured to generate the ionizable lipid candidate structures from the plurality of ionizable lipid structures by identifying ionizable lipid structures comprising cationic lipids.

In various embodiments, the processor is further configured to generate the ionizable lipid candidate structures from the plurality of ionizable lipid structures by identifying ionizable lipid structures comprising ionizable lipids with alkyl chains that are either below a first length threshold or above a second length threshold.

In various embodiments, the processor is further configured to generate the ionizable lipid candidate structures from the plurality of ionizable lipid structures by identifying ionizable lipid structures comprising a predetermined group of reagents.

In various embodiments, the machine learning model is selected from: Graph Neural Network (GNN), Convolutional Neural Network (CNN). Recurrent Neural Network (RNN), Transformer Model, Autoencoders, Capsule Network, Message Passing Neural Network (MPNN), Spectral CNN and Diffusion Neural Network.

In various embodiments, the processor is configured to train the machine learning model based on supervised, self-supervised, semi-supervised, unsupervised and/or reinforcement training.

In various embodiments, there is also included a method of synthesizing ionizable lipid structures comprises utilizing the one pot method for synthesizing an ionizable lipid of Formula I as described above and herein. Also included are compounds of Formula I and compositions comprising the compounds of Formula I, including LNP compositions, as well as various uses thereof.

Other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 is a block diagram of a system or platform for AI-guided ionizable lipid engineering in accordance with an example embodiment;

FIG. 2A is an example embodiment of a method for AI-guided ionizable lipid engineering;

FIG. 2B is another example embodiment of a method for AI-guided ionizable lipid engineering;

FIG. 2C is a further example of a method for AI-guided ionizable lipid engineering;

FIG. 2D is another example of a method for AI-guided ionizable lipid engineering;

FIG. 3 illustrates a block diagram of a virtual library design of ionizable lipid structures according to an example embodiment;

FIG. 4 is an example embodiment of a method for generating a library of ionizable lipid candidate structures;

FIG. 5 is an example embodiment of a method for ranking ionizable lipid candidate structures;

FIG. 6 illustrates a method for high-throughput synthesis for ionizable lipids according to an example embodiment;

FIG. 7 illustrates an example embodiment of a schematic diagram of a LNP for cargo molecule encapsulation;

FIG. 8 illustrates an example embodiment of a balloon plot of 1,200 LNPs for Fluc mRNA (mLuc) delivery and corresponding luciferase expression in Hela cells;

FIG. 9 illustrates an example embodiment of a plot of loss value on the training data set and validating data set against fine-tuning step;

FIG. 10 illustrates an example embodiment of a plot of mean square error (mse) scores;

FIG. 11 illustrates an example embodiment of a uniform manifold approximation and projection (UMAP) plot of 1,200 experimental LNPs based on transfection potency;

FIG. 12 illustrates an example embodiment of a plot of transfection efficiency of top 5 headgroups from the data of FIG. 8 in Raw 264.7 cells;

FIG. 13 illustrates an example embodiment of a plot of transfection efficiency of top 3 and bottom 2 tailgroups from the data of FIG. 8 in Raw 264.7 cells;

FIG. 14 illustrates an example embodiment of a plot of transfection efficiency of 15 lipid candidates selected for validation for Raw 264.7 cells;

FIG. 15 illustrates a schematic diagram of top-performing formulation parameters used in the optimization of R080 LNPs in Raw 264.7 cells, according to an example embodiment;

FIG. 16 illustrates a comparison plot of the Fluc-mRNA transfection efficiency of different LNPs in Raw 264.7 cells, according to an example embodiment;

FIG. 17 illustrates a comparison plot of the efficacy of AGILE-LNPs (H278 LNPs and R080 LNPs) in Hela cells, according to an example embodiment;

FIG. 18 illustrates confocal images of R080 LNP that was able to successfully deliver GFP mRNA to Raw 264.7 and expressing green fluorescent protein, according to an example embodiment;

FIG. 19A is an example embodiment of a method for AI-guided ionizable lipid engineering; and

FIG. 19B is another example embodiment of a method for AI-guided ionizable lipid engineering.

Further aspects and features of the example embodiments described herein will appear from the following description taken together with the accompanying drawings.

### DETAILED DESCRIPTION OF THE EMBODIMENTS

Various embodiments in accordance with the teachings herein will be described below to provide an example of at least one embodiment of the claimed subject matter. No embodiment described herein limits any claimed subject matter. The claimed subject matter is not limited to devices, systems, or methods having all of the features of any one of the devices, systems, or methods described below or to features common to multiple or all of the devices, systems, or methods described herein. It is possible that there may be a device, system, or method described herein that is not an embodiment of any claimed subject matter. Any subject matter that is described herein that is not claimed in this document may be the subject matter of another protective instrument, for example, a continuing patent application, and the applicants, inventors, or owners do not intend to abandon, disclaim, or dedicate to the public any such subject matter by its disclosure in this document.

It will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures, and components have not been described in detail so as not to obscure the embodiments described herein. Also, the description is not to be considered as limiting the scope of the embodiments described herein.

### Definitions

It should also be noted that the terms “coupled” or “coupling” as used herein can have several different meanings depending in the context in which these terms are used. For example, the terms coupled or coupling can have a mechanical or electrical connotation. For example, as used herein, the terms coupled or coupling can indicate that two elements or devices can be directly connected to one another or connected to one another through one or more intermediate elements or devices via an electrical signal, electrical connection, or a mechanical element depending on the particular context.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5%, or 10%, for example, if this deviation does not negate the meaning of the term it modifies.

Furthermore, the recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed, such as 1%, 2%, 5%, or 10%, for example.

It should also be noted that the use of the term “window” in conjunction with describing the operation of any system or method described herein is meant to be understood as describing a user interface for performing initialization, configuration, or other user operations.

The term “pharmaceutically acceptable” means compatible with the treatment of subjects.

The term “subject” as used herein includes all members of the animal kingdom including mammals, and suitably refers to humans. Thus, the therapeutic methods and uses of the present application are applicable to both human therapy and veterinary applications.

The term “administered” as used herein means administration of a therapeutically effective amount of one or more compounds or compositions as defined herein to a cell, tissue, organ or subject.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. Thus, for example, the term “C₁₋₆alkyl” (or “C₁-C₆alkyl”) means an alkyl group having 1, 2, 3, 4, 5, or 6 carbon atoms and includes, for example, any of the hexyl alkyl and pentyl alkyl isomers as well as n-, iso-, sec- and tert-butyl, n- and iso-propyl, ethyl and methyl. As another example, “C₁₋₄alkyl” refers to n-, iso-, sec- and tert-butyl, n- and isopropyl, ethyl and methyl.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cₙ₁₋ₙ₂”. For example, the term C₁₋₆alkylene means an alkylene group having 1, 2, 3, 4, 5 or 6 carbon atoms.

The term “alkenyl” whether it is used alone or as part of another group, means a straight or branched chain, unsaturated alkyl groups containing at least one double bond. The number of carbon atoms that are possible in the referenced alkenyl group are indicated by the prefix “Cₙ₁₋ₙ₂”. For example, the term C₂₋₆alkenyl means an alkenyl group having 2, 3, 4, 5 or 6 carbon atoms.

The term “heterocycloalkyl” as used herein, whether it is used alone or as part of another group, refers to cyclic groups containing a saturated ring in which one or more of the atoms are a heteromoiety selected from O, S, SO, SO₂, N, NH and NC₁₋₆alkyl and the remaining atoms are C.

The term “heterocycloalkenyl” as used herein, whether it is used alone or as part of another group, refers to cyclic groups containing an unsaturated ring (i.e., contain one or more double bonds) in which one or more of the atoms are a heteromoiety selected from O, S, SO, SO₂, N, NH and NC₁₋₆alkyl and the remaining atoms are C.

The term “hydrolyzable” as used herein refers to a chemical group that comprises at least one bond that is broken by a hydrolysis reaction under certain physiological conditions.

The term “hydrolysis” as used herein refers to a chemical reaction in which a molecule of water breaks one or more chemical bonds.

As used herein an “ionizable lipid” refers to a class of lipid molecules which remain neutral at physiological pH, but are protonated at low pH, making them positively charged. Therefore the term “ionizable” nitrogen atom refers to a nitrogen atom in a molecule that remains neutral at physiological pH, but becomes protonated at lower pH making it positively charged.

As used herein, the term “nanoparticle” refers to any particle having a diameter making the particle suitable for systemic, in particular intravenous administration, of active agents.

As used herein, the term “lipid nanoparticle” (LNP), also termed solid lipid nanoparticles (SLNP), refers to a nanoparticle comprising lipids. LNPs or SLNPs are often used as a pharmaceutical drug delivery system or pharmaceutical formulation.

The term “transfection efficiency” as used herein refers to the proportion or percentage of cells within a population that successfully uptake the cargo molecule. When the cargo molecule is mRNA, the term “transfection efficiency” as used herein refers to the proportion or percentage of cells within a population that successfully uptake and express the mRNA, introduced into them via the LNP.

The example embodiments of the devices, systems, or methods described in accordance with the teachings herein may be implemented as a combination of hardware and software. For example, the embodiments described herein may be implemented, at least in part, by using one or more computer programs, executing on one or more programmable devices comprising at least one processing element and at least one storage element (i.e., at least one volatile memory element and at least one non-volatile memory element). The hardware may comprise input devices including at least one of a touch screen, a keyboard, a mouse, buttons, keys, sliders, and the like, as well as one or more of a display, a printer, and the like depending on the implementation of the hardware.

It should also be noted that there may be some elements that are used to implement at least part of the embodiments described herein that may be implemented via software that is written in a high-level procedural language such as object-oriented programming. The program code may be written in C++, C#, JavaScript, Python, or any other suitable programming language and may comprise modules or classes, as is known to those skilled in object-oriented programming. Alternatively, or in addition thereto, some of these elements implemented via software may be written in assembly language, machine language, or firmware as needed. In either case, the language may be a compiled or interpreted language.

At least some of these software programs may be stored on a computer readable medium such as, but not limited to, a ROM, a magnetic disk, an optical disc, a USB key, and the like that is readable by a device having a processor, an operating system, and the associated hardware and software that is necessary to implement the functionality of at least one of the embodiments described herein. The software program code, when read by the device, configures the device to operate in a new, specific, and predefined manner (e.g., as a specific-purpose computer) in order to perform at least one of the methods described herein.

At least some of the programs associated with the devices, systems, and methods of the embodiments described herein may be capable of being distributed in a computer program product comprising a computer readable medium that bears computer usable instructions, such as program code, for one or more processing units. The medium may be provided in various forms, including non-transitory forms such as, but not limited to, one or more diskettes, compact disks, tapes, chips, and magnetic and electronic storage. In alternative embodiments, the medium may be transitory in nature such as, but not limited to, wire-line transmissions, satellite transmissions, internet transmissions (e.g., downloads), media, digital and analog signals, and the like. The computer useable instructions may also be in various formats, including compiled and non-compiled code.

### Methods for Preparing Ionizable Lipids, Including Combinatorial Methods Therefore and Lipids Prepared Using the Method

The success of messenger RNA (mRNA) vaccines developed to combat the severe acute respiratory syndrome coronavirus 2 (SARS-COV-2) pandemic has catalyzed a surge of interest in mRNA-based therapies (1, 2). Many applications, spanning prophylactic and therapeutic vaccines, protein replacement therapies, cancer immunotherapies, cellular reprogramming, and gene editing, are under clinical investigation (3). However, an overarching challenge persists for the effective and safe delivery of cargo molecules, such as mRNA to the cytoplasm of target cells. Three hurdles to be addressed with respect to large biomolecules such as mRNA include: rapid degradation and lack of localization of the cargo, especially sensitive biomolecules; impeded passive uptake due to the large size, and in particular for RNA, its polyanionic nature (4); and the necessity for safe, well-tolerated, and body-clearable delivery vehicles (5, 6). Currently, ionizable lipid nanoparticles (LNPs) are widely employed as the delivery vehicle for cargo molecules such as mRNA with demonstrated efficacy and safety profiles in clinical practice. Thus far, LNPs have enabled three FDA-approved RNA-based therapies, including an siRNA drug (Onpattro) (7) and two mRNA vaccines against SARS-COV-2 (Comirnaty and Spikevax) (8, 9). It's noteworthy that numerous mRNA-LNP therapies are progressing through various clinical trial stages (10), including the first-ever CRISPR/Cas9-based therapy approved for in-human testing (ClinicalTrials.gov Identifier: NCT05120830) (11). While LNPs have been instrumental in clinical translation of mRNA, many designs can result in substantial mRNA expression in the liver, potentially causing reversible liver damage. This effect has been documented in the case of Pfizer's COVID-19 vaccine (BNT162b2), which upon intramuscular administration, predominantly localizes to the liver and the injection site, leading to undesirable hepatic injury (12). On the other hand, studies have shown that mRNA-free LNPs do not inflict liver damage (13), suggesting that the safety issues associated with mRNA vaccines might arise from mRNA expression in non-target tissues. Hence, there is an need for LNPs designed for tissue-specific delivery, minimizing off-target effects and addressing safety issues associated with current LNPs.

The classical LNP formulation consists of four components: ionizable lipids, helper phospholipids, cholesterol, and lipid-conjugated polyethylene glycol (PEGylated lipids) (14). Of these, the ionizable lipid is pivotal in this complex as it not only stabilizes cargo molecules such as mRNA via electrostatic interactions but also promotes endosome escape, facilitating efficient cytosolic delivery (15, 16). During formulation, the amine headgroups of ionizable lipids acquire positive charge under acidic conditions, thus favoring the encapsulation of negatively charged molecules into LNPs. The remaining, unbound ionizable lipids then revert to neutrality during buffer exchange at physiological pH, preventing potential toxicity observed upon non-ionizable cationic lipids (17-20). Post endocytosis by target cells, ionizable lipids are protonated again in the acidic endosomal environment, allowing them to interact with the negatively charged endosomal membrane. This facilitates the disruption of both LNP and endosome, ultimately leading to the release of cargos into the cytoplasm. The importance of ionizable lipids is underscored by the fact that all three FDA approved RNA-LNP drugs utilize unique ionizable lipids in their formulations. Therefore, innovating ionizable lipid design is crucial for the development of LNPs with enhanced performance, for example, in muscle-specific mRNA delivery.

While previous research has shed light on the rational design of ionizable lipids, their synthesis remains a complex, time-consuming, and costly process. This necessitates rapid, streamlined synthetic approaches.

Towards this goal, combinatorial chemistry, employing multi-component reactions, has emerged as an effective tool for high-throughput synthesis (HTS) of expansive and chemically diverse lipid libraries. Typical examples include a Ugi-based three-component reaction (3-CR), which has been demonstrated to expedite the synthesis of a combinatorial library of ionizable lipids within 24 hours at room temperature, aiding in the discovery of a STING-activating ionizable lipid beneficial for mRNA vaccine delivery (21). Despite the potential, ionizable lipids synthesized using this approach suffer from inconsistent and poor yield (3.9% to 34%). Such low yields complicate high throughput analysis as the presence of impurities and low-yielding potent ionizable lipids in LNP formulations can obscure their genuine potential. Additionally, ionizable lipids generated by this method lack biodegradable bonds in the lipid tails, which improve the safety profiles of ionizable lipids, increasing the therapeutic window, and enabling repeated dosing of LNPs (22, 23). Rodent studies have shown that LNPs containing non-biodegradable ionizable lipids can cause liver damage (24-26), underlining the need for biodegradable lipid design.

To address this, a Michael Addition-based 3-CR method was reported that generates biodegradable ionizable lipids. This led to the discovery of a lipid uniquely suited for efficient mRNA delivery to the lung epithelium (27). Nevertheless, the applicability of this approach is hampered by its protracted 72-hour duration and the need for heat. Furthermore, ionizable lipids created by both known 3-CR methods were restricted to possessing symmetric tails. Notably, the asymmetry of ionizable lipid tails has been increasingly recognized for facilitating endosomal release and cytosolic payload delivery (28). This is attributed to the pronounced difference in their stretch and bending moduli of asymmetric ionizable lipids significantly compared to naturally occurring symmetric phospholipids, enhancing their efficacy in disrupting the endosomal membranes (28). Consequently, there is a prominent need, yet unmet, for a streamlined HTS platform capable of swiftly synthesizing biodegradable, asymmetrical lipids with a high yield.

As will be described in greater detail herein below, in accordance with the teachings herein, there are provided various embodiments of AI-based systems and methods for developing ionizable lipid nanoparticles for cargo molecule delivery, including mRNA. LNPs for delivering therapeutic cargo molecules, such as, for example, COVID-19 mRNA vaccines, can oftentimes inadvertently transfect off-target tissues, leading to potential safety issues. Disclosed herein are AI-based systems and methods to develop chemically diverse lipids for building LNPs that can facilitate tissue-specific delivery with improved precision.

The various systems and methods disclosed herein include an advanced Ugi-based 3-CR platform that enables the one-pot, high-yield fabrication of biodegradable, asymmetrical ionizable lipids at room temperature. Further, the various systems and methods disclosed herein strategically reconfigure functional groups and optimize component design to enhance the chemical diversity of the resulting lipids, particularly for their lipid tails.

Therefore, in accordance with the teachings herein there is included a one pot method of preparing an ionizable lipid of Formula I:

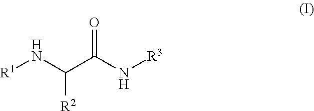

comprising reacting a compound of Formula A, a compound of Formula B and a compound of Formula C:

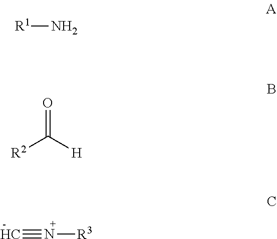

in the presence of a catalyst under conditions to provide the compound of Formula I,
wherein:
R¹ is a cyclic or acyclic group comprising at least one ionizable nitrogen atom,
R² is selected from C₁₋₃₀alkyl and C₂₋₃₀alkenyl, each of which is optionally interrupted by a hydrolysable group; and
R³ is selected from C₁₋₃₀alkyl and C₂₋₃₀alkenyl, each of which is optionally interrupted by a hydrolyzable group.

In some embodiments, R¹ is selected from C₁₋₆alkyleneNR⁴R⁵ and 5- to 8-membered heterocycloalkyl and 5- to 8-membered heterocycloalkenyl, wherein R⁴ and R⁵ are independently selected from H, C₁₋₆alkyl and C₁₋₆alkyl substituted with one or two NR⁶R⁷ and R⁶ and R⁷ are independently selected from H and C₁₋₆alkyl, or R⁴ and R⁵, together with the nitrogen atom to which they are attached, form 5- or 6-membered heterocycloalkyl or 5- to 6-membered heterocycloalkenyl.

In some embodiments, R¹ is selected from C₁₋₆alkyleneNR⁴R⁵ and 5- to 6-membered heterocycloalkyl and 5- to 8-membered heterocycloalkenyl, wherein R⁴ and R⁵ are independently selected from H, C₁₋₄alkyl and C₁₋₄alkyl substituted with one NR⁶R⁷ and R⁶ and R⁷ are independently selected from H and C₁₋₄alkyl, or R⁴ and R⁵, together with the nitrogen atom to which they are attached, form 5- or 6-membered heterocycloalkyl or 5- to 6-membered heterocycloalkenyl.

In some embodiments, the 5- or 6-membered heterocycloalkyl or 5- to 6-membered heterocycloalkenyl comprises one or two additional heteromoieties selected from O, NH and NC₁₋₄alkyl. In some embodiments, the 5- or 6-membered heterocycloalkyl or 5- to 6-membered heterocycloalkenyl comprises one additional heteromoieties selected from O, NH and NCH₃.

In some embodiments R¹ is selected from:

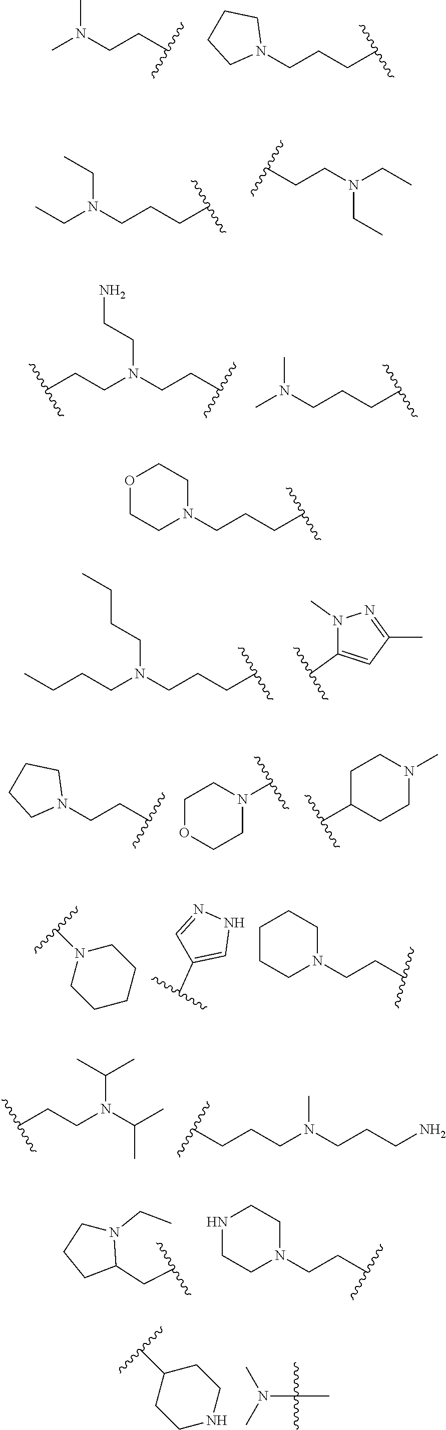

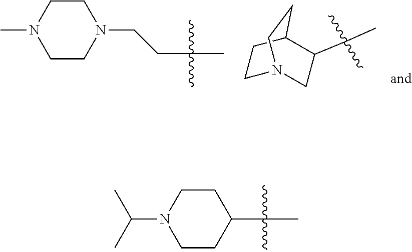

In some embodiments R² is selected from C₁₋₂₀alkyl and C₂₋₂₀alkenyl, each of which is optionally interrupted by a hydrolyzable group. In some embodiments, the C₂₋₂₀alkenyl in R² comprises one double bond.

In some embodiments R³ is selected from C₁₋₂₀alkyl and C₂₋₂₀alkenyl, each of which is optionally interrupted by a hydrolyzable group. In some embodiments, the C₂₋₂₀alkenyl in R³ comprises one double bond.

In some embodiments, one of R² and R³ comprises an alkyl or an alkenyl group that is interrupted by a hydrolyzable group. In some embodiments, R² comprises an alkyl or an alkenyl group that is interrupted by a hydrolyzable group. In some embodiments, the hydrolyzable group is selected from O, OC(O) and NHC(O). In some embodiments, the hydrolyzable group is OC(O).

In some embodiment, R² and R³ are different.

In some embodiments, R² is selected from:

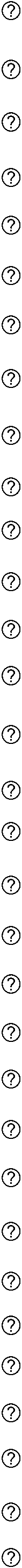

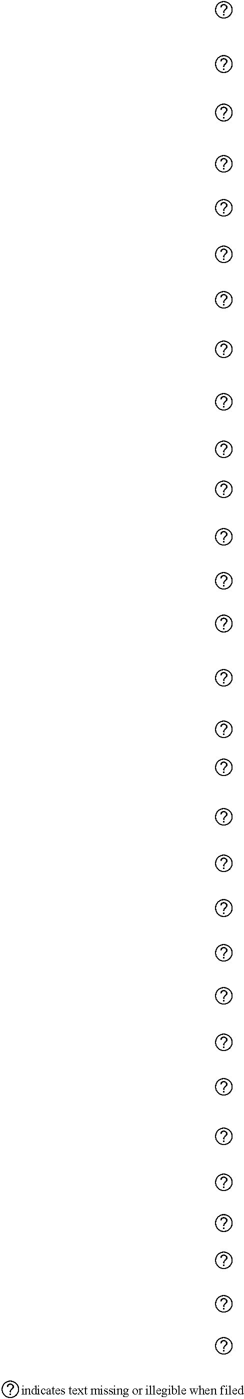

In some embodiments, R² is selected from:

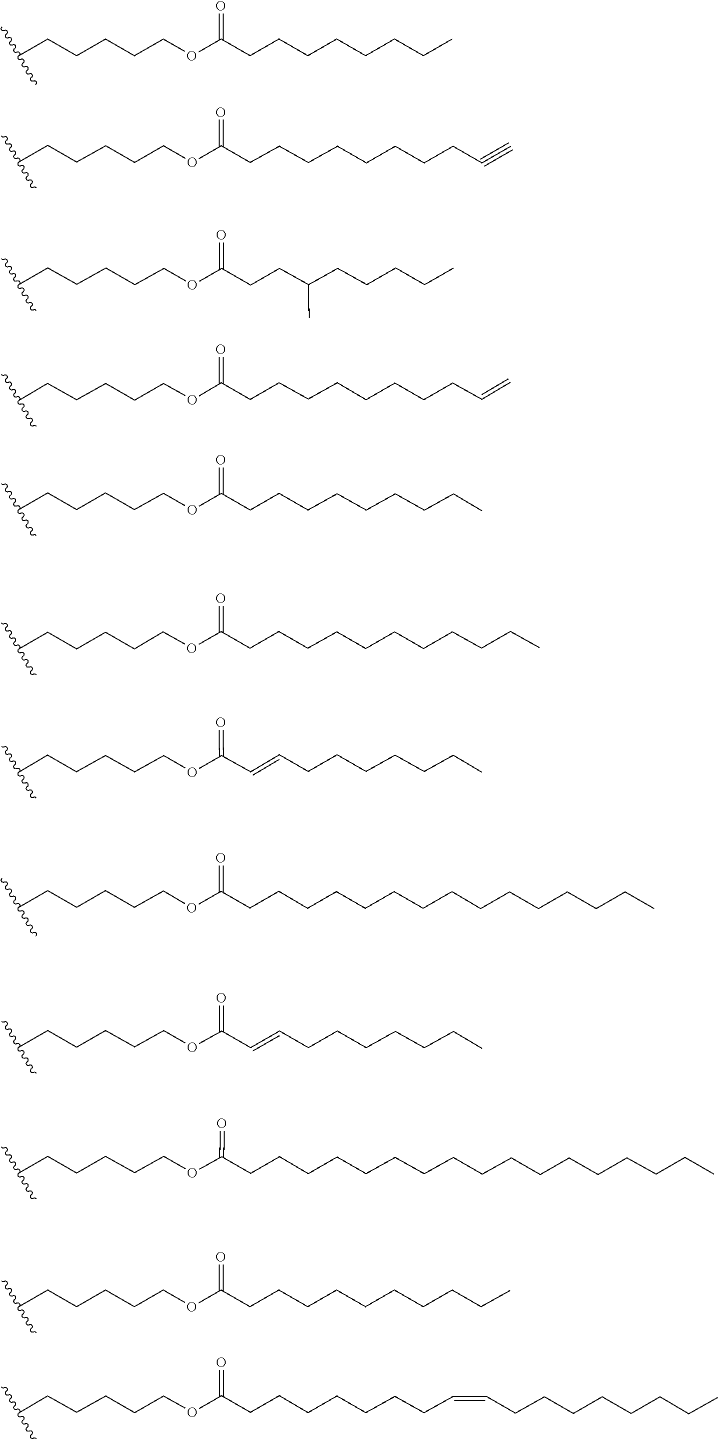

In some embodiments, R³ is selected from:

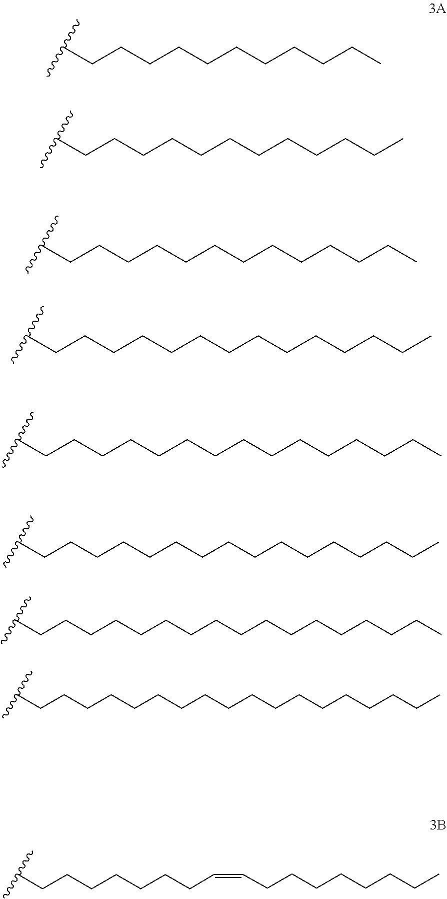

In some embodiments, R³ is selected from:

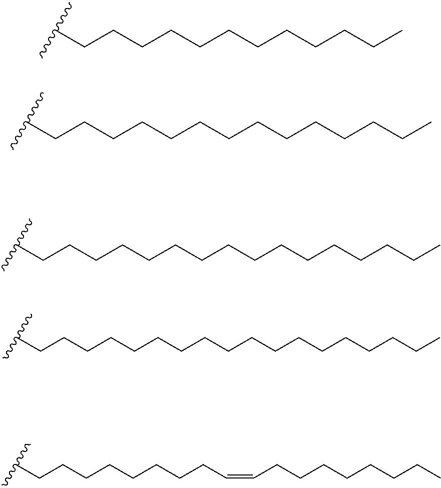 [SMILES: *CCCCCCCCCCCC ; *CCCCCCCCCCCCCC ; *CCCCCCCCCCCCCCCC ; *CCCCCCCCCCCCCCCCCC ; *CCCCCCCC/C=C\CCCCCCCC]

In some embodiments, the compounds of Formula A, Formula B and Formula C are reacted in a molar ratio of about 1:1:1.

In some embodiments, the catalyst is a Lewis acid. In some embodiments, the catalyst is selected from toluene sulfonic acid (TsOH), phosphoric acid (H₃PO₄), perboric acid (H₃BO₄) and phenylphosphinic acid (PhP(O)(H)(OH)). In some embodiments, the catalyst is phenylphosphinic acid.

In some embodiments, the catalyst is used in an amount of about 10 mol % to about 30 mol %, suitably 20 mol %.

In some embodiments, the under conditions to provide the compound of Formula I comprise use of an alcoholic solvent, such as methanol and a reaction temperature of about 20° C. to about 25° C. (room temperature) for about 6 hours to about 48 hours or about 12 hours to about 24 hours.

In some embodiments, the one pot method of preparing an ionizable lipid of Formula I is performed using combinatorial chemistry procedures. Therefore, in some embodiments, the method comprises performing each reaction in an individual well of a multiwell plate and the addition of each reagent, e.g. the compounds of Formula A, Formula B and Formula C, as well as the catalyst and any solvent, are added to the plates using automated systems known in the art. In some embodiments, the compounds of Formula I are not isolated prior to use, for example, for preparation of lipid nanoparticles (LNPs).

In some embodiments, the methods disclosed herein for the preparation of compounds of Formula I have demonstrated a marked improvement in the yield of ionizable lipids by consistently achieving a reaction efficiency exceeding 70% through the incorporation of a non-toxic catalyst, such as phenylphosphinic acid. The methods disclosed herein advantageously provide access to ionizable lipids having two different lipid tails (asymmetrical).

In accordance with the teachings herein, there is also included ionizable lipids of Formula (I), and salts thereof,

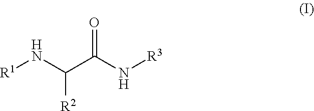

wherein:
R¹ is a cyclic or acyclic group comprising at least one ionizable nitrogen atom,
R² is selected from C₁₋₃₀alkyl and C₂₋₃₀alkenyl, each of which is optionally interrupted by a hydrolysable group; and
R³ is selected from C₁₋₃₀alkyl and C₂₋₃₀alkenyl, each of which is optionally interrupted by a hydrolyzable group.

In some embodiments, R¹ is selected from C₁₋₆alkyleneNR⁴R⁵ and 5- to 8-membered heterocycloalkyl and 5- to 8-membered heterocycloalkenyl, wherein R⁴ and R⁵ are independently selected from H, C₁₋₆alkyl and C₁₋₆alkyl substituted with one or two NR⁶R⁷ and R⁶ and R⁷ are independently selected from H and C₁₋₆alkyl, or R⁴ and R⁵, together with the nitrogen atom to which they are attached, form 5- or 6-membered heterocycloalkyl or 5- to 6-membered heterocycloalkenyl.

In some embodiments, R¹ is selected from C₁₋₆alkyleneNR⁴R⁵ and 5- to 6-membered heterocycloalkyl and 5- to 6-membered heterocycloalkenyl, wherein R⁴ and R⁵ are independently selected from H, C₁₋₄alkyl and C₁₋₄alkyl substituted with one NR⁶R⁷ and R⁶ and R⁷ are independently selected from H and C₁₋₄alkyl, or R⁴ and R⁵, together with the nitrogen atom to which they are attached, form 5- or 6-membered heterocycloalkyl or 5- to 6-membered heterocycloalkenyl.

In some embodiments, the 5- or 6-membered heterocycloalkyl or 5- to 6-membered heterocycloalkenyl comprises one or two additional heteromoieties selected from O, NH and NC₁₋₄alkyl. In some embodiments, the 5- or 6-membered heterocycloalkyl or 5- to 6-membered heterocycloalkenyl comprises one additional heteromoieties selected from O, NH and NCH₃.

In some embodiments, the compounds of Formula I are selected from:

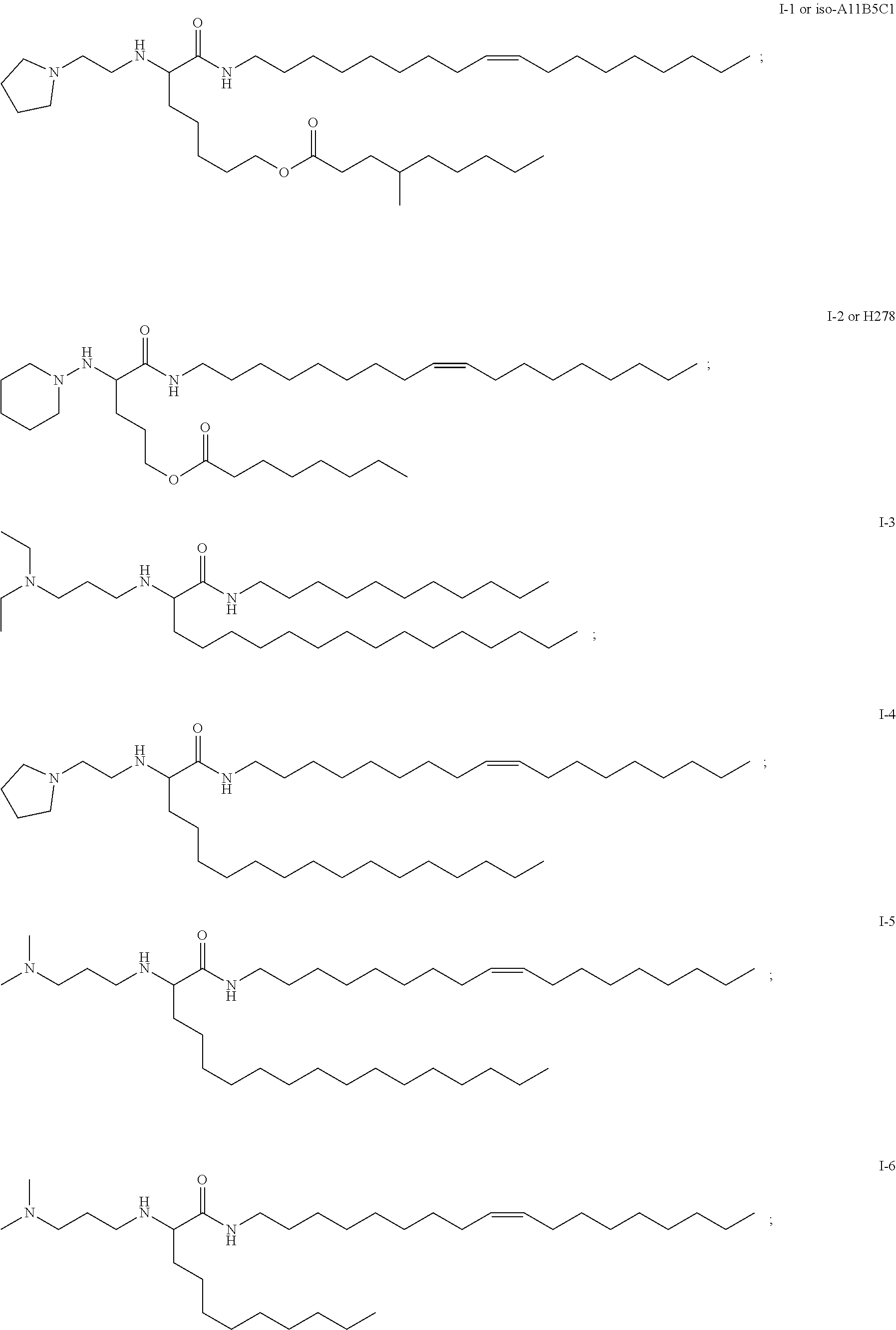

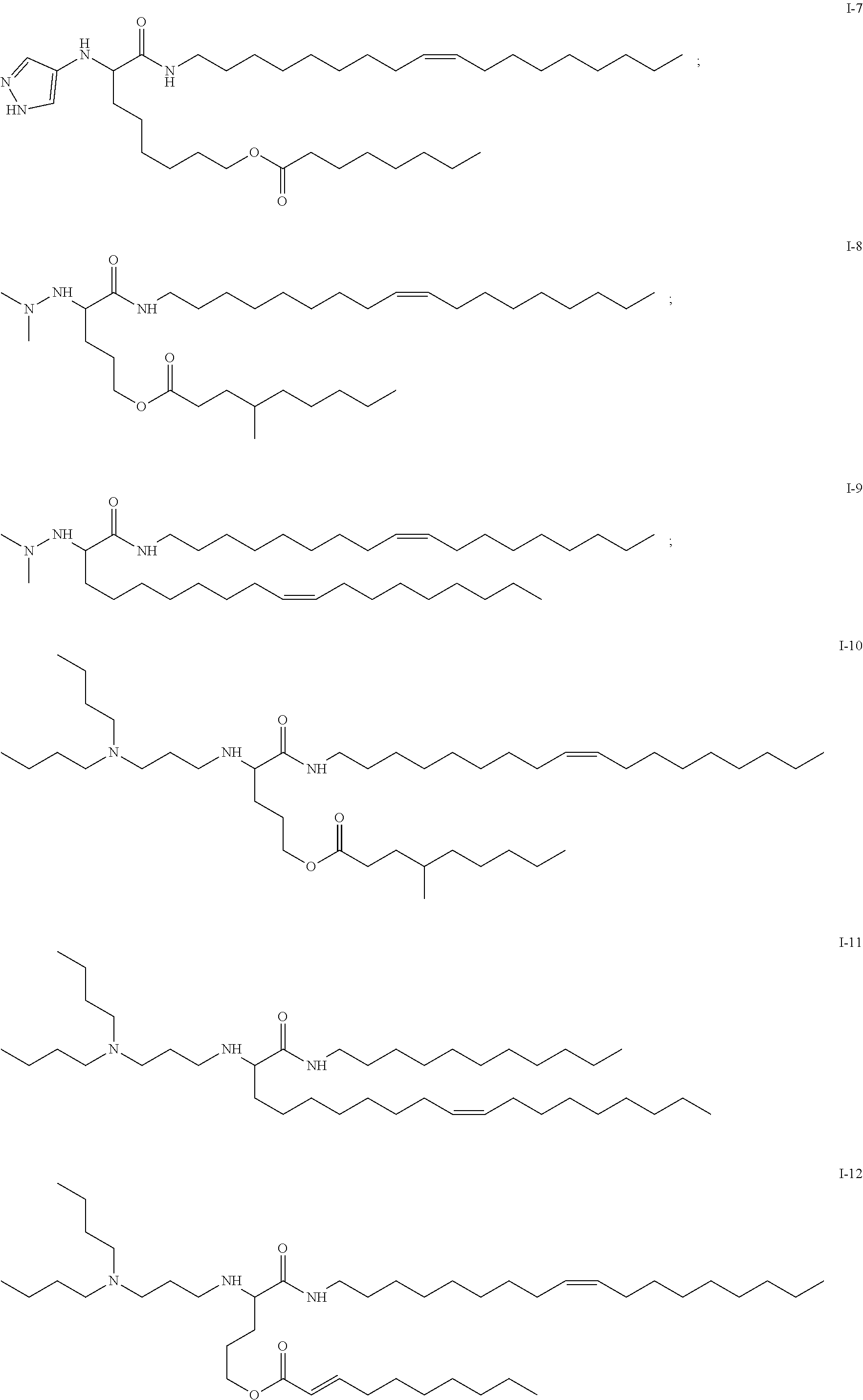

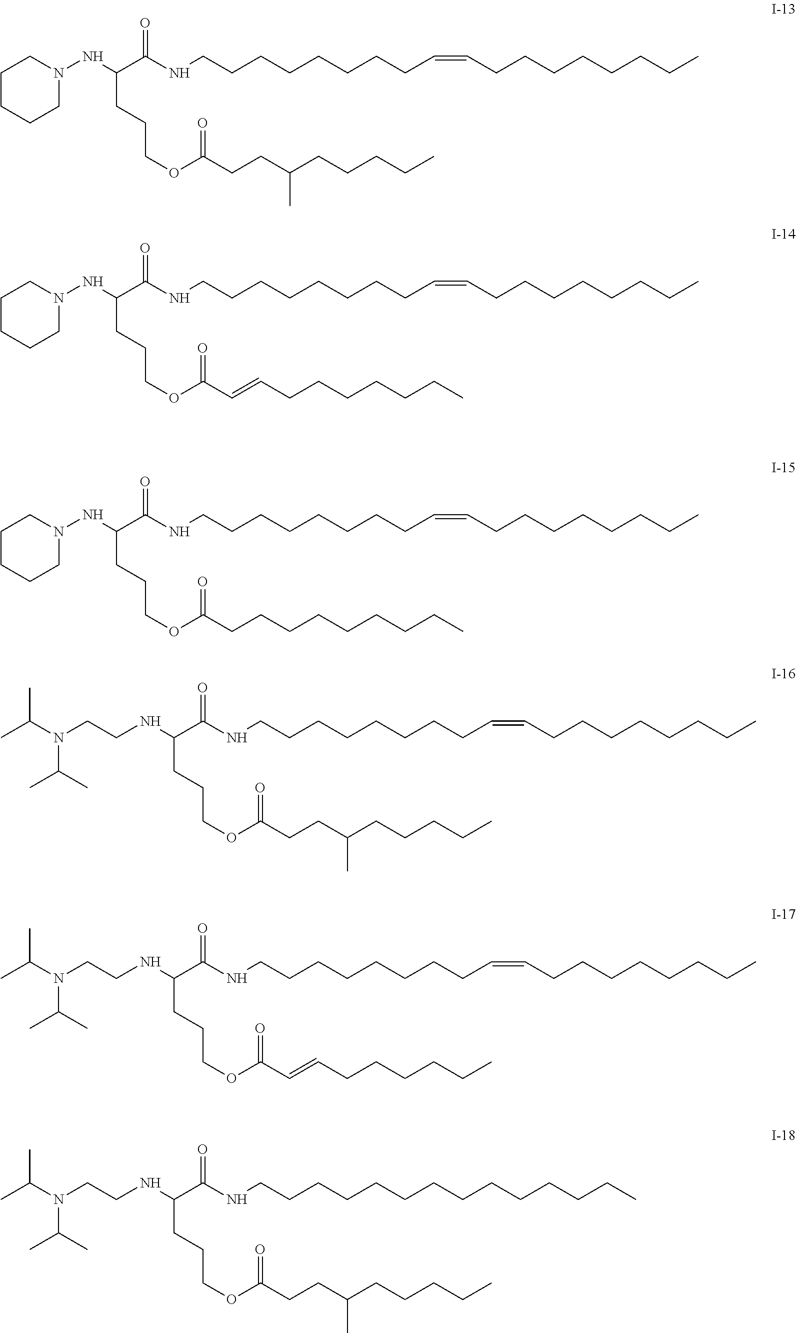

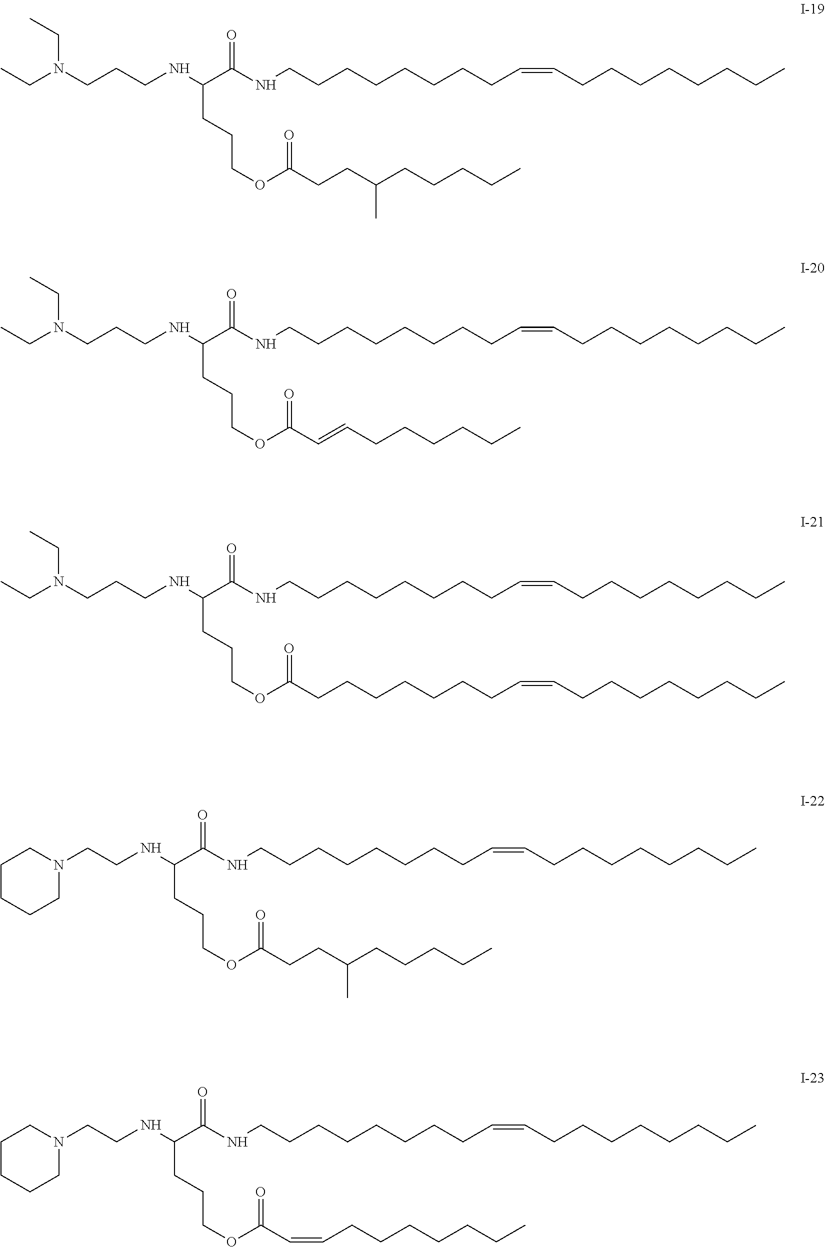

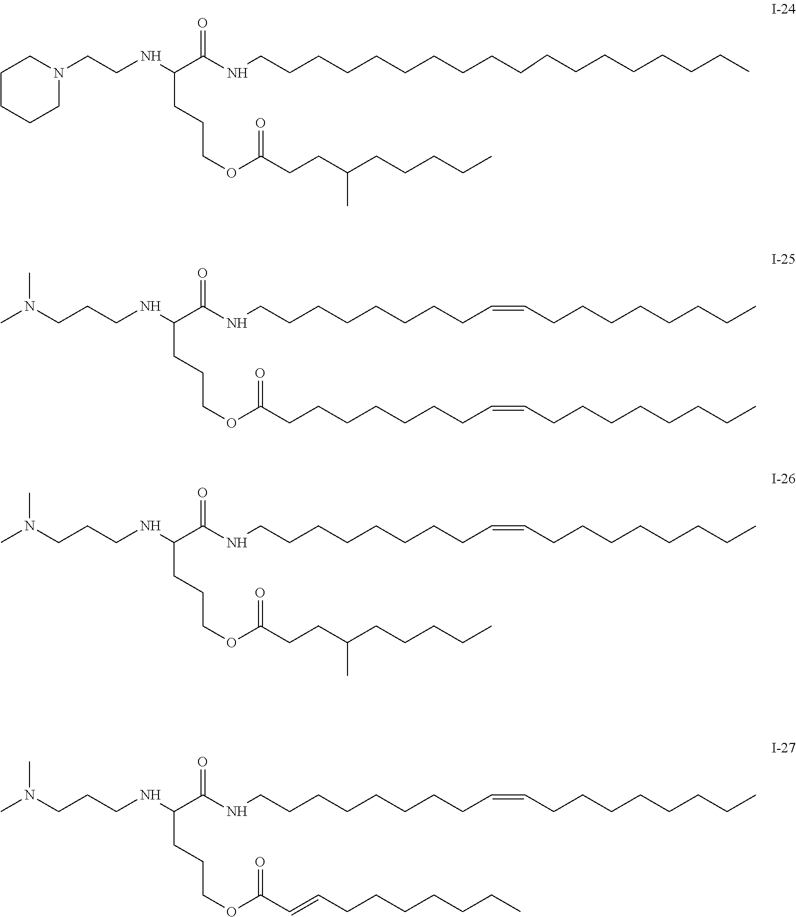

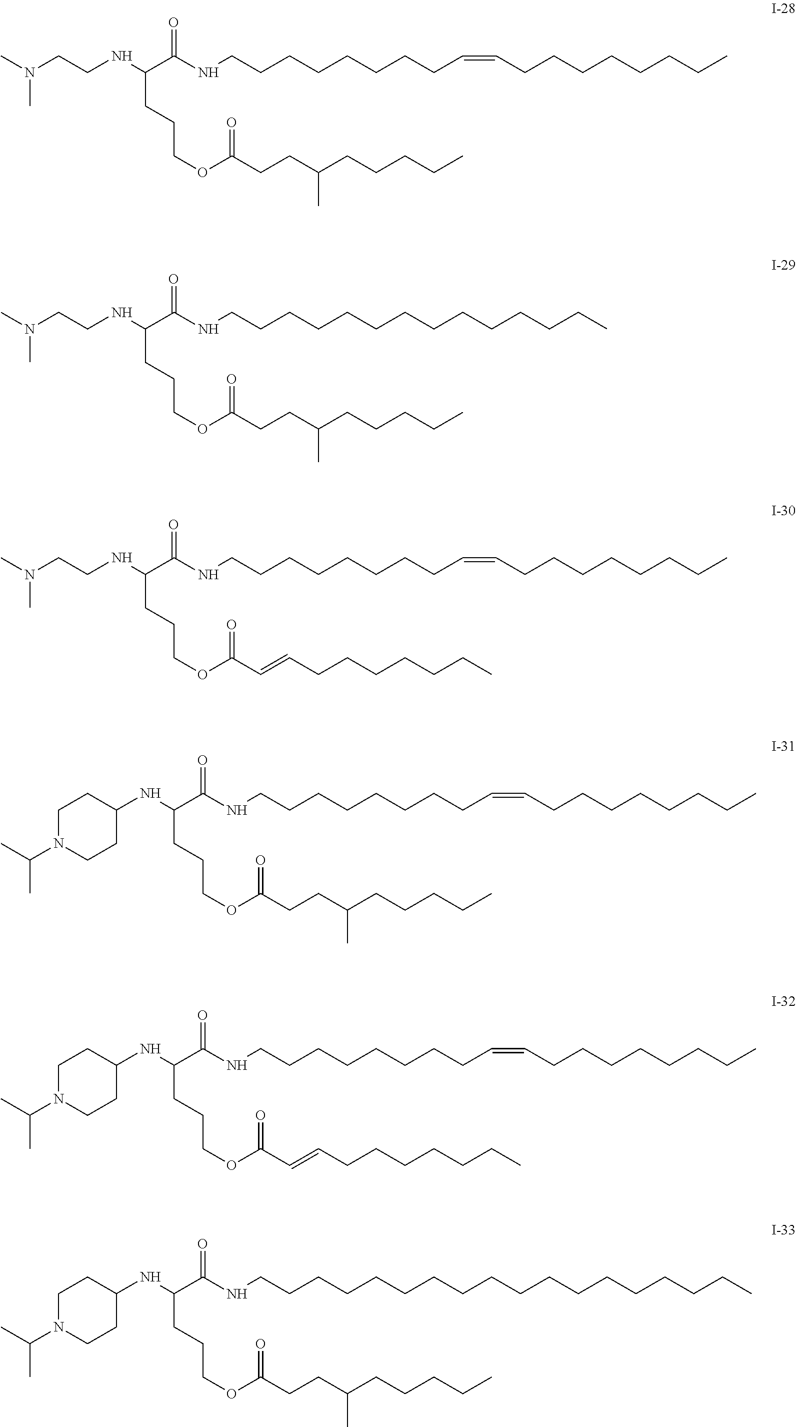

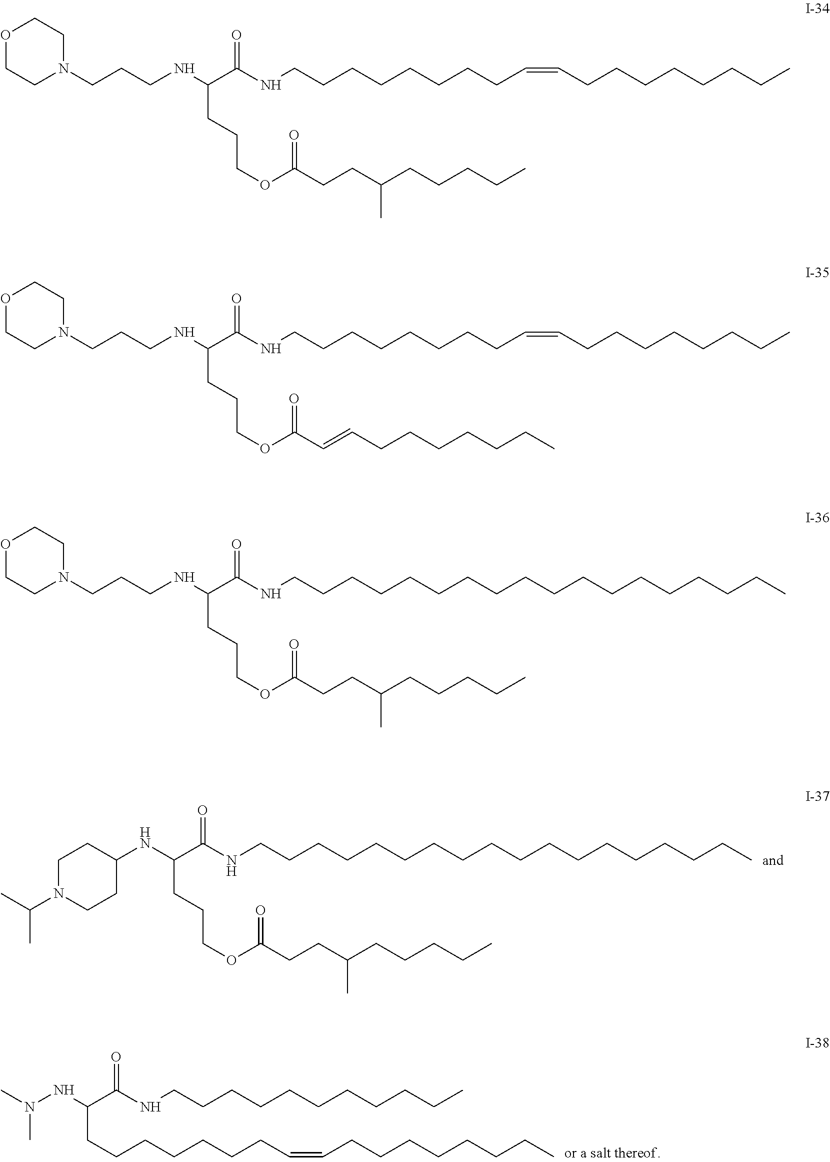

In some embodiments, the ionizable lipids of Formula I exist as different isomers. In particular the lipid chains may exist in the trans or cis configuration such as when they contain double bonds. In some embodiments, the lipids as defined herein occur in the cis configuration.

As used herein, when a compound has one or more stereocenters, each stereocenter may have the R or S configuration, unless stated otherwise. In some embodiment the compound therefore is a racemic mixture of enantiomers and/or diastereoisomers, or in some embodiments, it exists as a mixture have an excess of one or more of the enantiomers and/or diastereoisomers, such as more than 60%, more than 70%, more than 80%, more than 85%, more than 90%, more than 95%, more than 98%, more than 99%.

In some embodiments, the compound of Formula I is iso-A11B5C1 (I-A), with an mRNA delivery potency comparable to SM-102. LNPs with iso-A11B5C1 incorporated in them enable efficient muscle-focused mRNA delivery while minimizing off-target delivery to other tissues and organs, like liver and spleen. Further, the potential of iso-A11B5C1 LNPs in muscle-specific gene editing and its promise as a safe and effective delivery vehicle for cancer mRNA vaccines has also been demonstrated herein.

Despite the iso-A11B5C1's exhibited reduced mRNA transfection potency in lymph nodes and antigen-presenting cells (APCs), iso-A11B5C1-mediated mRNA vaccination through intramuscular injection still has the potential to induce robust cellular immune responses and demonstrate potent anti-tumor effect in a melanoma model, providing key insights into the mechanisms of mRNA vaccines and establishing its potential for future therapeutic mRNA vaccine development.

The teachings in this application provide the advantage of enriching the high throughput toolkit for generating tissue-specific ionizable lipids as well as encouraging a re-assessment of prevailing paradigms in mRNA vaccine design. The teachings herein further provide the advantage of advancing re-thinking of mRNA vaccine design principles, suggesting that achieving high immune cell transfection might not be the sole criterion for developing effective mRNA vaccines.

In some embodiments, there is also included herein a lipid nanoparticle or lipid nanoparticle composition comprising a lipid, in particular an ionizable lipid as defined herein, for example of Formula I or as identified using the AI-based systems and methods disclosed herein.

As noted above, the term “nanoparticle” refers to any particle having a diameter making the particle suitable for systemic, in particular intravenous administration, of active agents typically having a diameter of less than 1000 nanometers (nm), less than 500 nm, less than 200 nm, such as for example between 50 and 200 nm; or between 80 and 160 nm.

In some embodiments, embodiment, the nanoparticle is in the form of a lipid nanoparticle (LNP) or LNP composition comprising a lipid, in particular an ionizable lipid as defined herein.

A lipid nanoparticle is typically spherical with an average diameter between 10 and 1000 nanometers and possesses a lipid core matrix that can solubilize lipophilic molecules. The term lipid is used here in a broader sense and includes triglycerides, diglycerides, monoglycerides, fatty acids, steroids (e.g. cholesterol) and waxes. Biological membrane lipids such as phospholipids, c embodiments, the nucleic acid is deoxyribonucleic acid, ribonucleic acid, recombinantly produced and chemically synthesized molecules. In some embodiments, the nucleic acid is DNA, genomic DNA, cDNA, RNA, tRNA, mRNA, small interfering RNA (siRNA), circRNA, micro RNA (miRNA), antisense oligonucleotides, ribozymes, plasmids, immune stimulating nucleic acids, antisense nucleic acids, antagomirs (anti-miRs), miRs, supermiRs, U1 adaptors, and aptamers. In some embodiments, the nucleic acid is siRNA, tRNA, circRNA or mRNA, or a combination thereof. In some embodiments, the nucleic acid is mRNA.

A nucleic acid is either in the form of a molecule which is single stranded or double stranded and linear or closed covalently to form a circle. In some embodiments the nucleic acid is used for introduction into, i.e. transfection of cells, for example, in the form of RNA which can be prepared by in vitro transcription from a DNA template. The RNA can moreover be modified before application by stabilizing sequences, capping, and/or polyadenylation.

The term “RNA” relates to a molecule which comprises ribonucleotide residues and in some embodiments being entirely or substantially composed of ribonucleotide residues. “Ribonucleotide” relates to a nucleotide with a hydroxyl group at the 2′-position of a β-D-ribofuranosyl group. The term includes double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs. In some embodiments, nucleic acids are comprised in a vector. The vector may be any selected from vectors known to the skilled person including plasmid vectors, cosmid vectors, phage vectors such as lambda phage, viral vectors such as adenoviral or baculoviral vectors, or artificial chromosome vectors such as bacterial artificial chromosomes (BAC), yeast artificial or analogs of naturally-occurring RNA.

In some embodiments the term “RNA” relates to “mRNA” which means “messenger RNA” and relates to a “transcript” which is produced using DNA as template and encodes a peptide or protein. mRNA typically comprises a 5′ untranslated region (5′-UTR), a protein or peptide coding region and a 3′ untranslated region (3′-UTR). mRNA has a limited halftime in cells and in vitro. In some embodiments, mRNA is produced by in vitro transcription using a DNA template. In some embodiments, the RNA is obtained by in vitro transcription or chemical synthesis. The in vitro transcription methodology is known to the skilled person. For example, there is a variety of in vitro transcription kits commercially available.

In a further aspect, also included herein is a pharmaceutical composition comprising one or more nanoparticles as defined herein and a pharmaceutically acceptable agent, such as a carrier, excipient, etc.

Such pharmaceutical compositions are particularly suitable in various fields such as prophylactic vaccines, therapeutic vaccines, protein replacement therapies, gene editing, gene silencing, small molecule delivery and the like.

In some embodiments, the nanoparticles and pharmaceutical compositions as defined herein are used in the induction of an immune response in a subject by administering to the subject a pharmaceutical composition wherein the active agent is an immunostimulatory oligonucleotide. Therefore in some embodiments, a vaccine comprising one or more nanoparticles as defined herein is also included. In some embodiments, the vaccine comprises at least one active agent, such as a nucleic acid molecule, e.g. mRNA molecule encoding an antigen to which an adaptive immune response is mounted. In some embodiments, the antigen is present as a weakened or killed form of a microbe, a protein or peptide, or an antigen encoding a nucleic acid, such as a disease-associated antigen for example a tumor antigen. In some embodiments, the vaccines are prophylactic (for example: to prevent or ameliorate the effects of a future infection by any natural or “wild” pathogen), or therapeutic (example, to actively treat or reduce the symptoms of an ongoing disease).

In some embodiments, the nanoparticles and pharmaceutical compositions as defined herein are used in the treatment of a disease, condition or disorder characterized by the overexpression of a polypeptide in a subject by administering to the subject a pharmaceutical composition as defined herein wherein the active agent is a nucleic acid selected from an siRNA, a microRNA, and an antisense oligonucleotide, and wherein the siRNA, microRNA, or antisense oligonucleotide includes a polynucleotide that specifically binds to a polynucleotide that encodes the polypeptide, or a complement thereof. In some embodiments, the nucleic acid is a siRNA or miRNA.

In some embodiments, the nanoparticles and pharmaceutical compositions as defined herein are used in the treatment of a disease, condition or disorder characterized by underexpression of a polypeptide in a subject by administering to the subject a pharmaceutical composition as defined herein, wherein the active agent is a plasmid that encodes the polypeptide or a functional variant or fragment thereof, such as in the context of protein replacement therapy.

In some embodiments, the nanoparticles and compositions as defined herein are used as a transfection agent that includes the compositions or nanoparticles described herein, wherein the composition or nanoparticles comprise a nucleic acid. The agent, when contacted with cells, can efficiently deliver nucleic acids to the cells. Yet another aspect is a method of delivering a nucleic acid to the interior of a cell, by obtaining or forming a composition or nanoparticles described herein, and contacting the composition or lipid particles with a cell.

### AI-Guided Ionizable Lipid Engineering (AGILE) for Lipid Nanoparticle (LNP) Based Cargo Molecule Delivery

As noted above, innovative ionizable lipid design is important for the development of LNPs with enhanced performance in cargo molecule delivery, including, intramuscular delivery, T-cell delivery, stem cell delivery, or any other tissue or cell specific delivery.

However, as mentioned above, designing effective ionizable lipids remains a challenging and time-consuming task due to the limited understanding of their physiochemical properties, necessitating comprehensive in vitro and in vivo testing. Additionally, the efficacy of delivery can differ among various cell lines and tissue, even when utilizing the same ionizable lipid.

Reference is made herein to an AI-Guided ionizable lipid engineering (AGILE) platform that aims to address the above-noted issues. AGILE platform provides various advantages for rapid ionizable lipid design, such as, for example, i) high-throughput lipid synthesis and screening using combinatorial chemistry; ii) large-scale in-silico screening via deep neural networks; and iii) easy customization to different cells and tissues.

At least one new ionizable lipid resulting from the systems and methods disclosed herein is a H278. The H278-mediated LNP, for use for intramuscular injection, identified from approximately 12,000 candidates demonstrated superior mRNA transfection efficiency compared to MC3 LP.

At least one other new ionizable lipid resulting from the systems and methods disclosed herein is iso-A11B5C1 (compound I-1). The iso-A11B5C1-mediated LNP, for use for intramuscular injection, thus identified demonstrated an mRNA delivery potency comparable to SM-102.

The AGILE platform has also resulted in the discovery of highly efficient LNPs for delivery to macrophages, which demonstrated the potential of the AGILE platform to customize LNPs for specific cells.

In various embodiments disclosed herein, the AGILE-platform includes the use of combinatorial chemistry to generate an extensive and chemically diverse lipid library for high-throughput screening the data from which enhances the identification of exceptional ionizable lipids. In some embodiments, the combinatorial chemistry comprises the method for the preparation of ionizable lipids as described above.

Generally examining thousands of LNPs generated by a combinatorial lipid library for mRNA delivery remains a laborious, time-consuming, and costly endeavor. Additionally, the ionizable lipid design space is still constrained by the headgroups and tailgroups employed in the production of the combinatorial lipid library. The AI-based systems and methods disclosed herein address these challenges and accelerate the discovery and optimization of ionizable lipids for efficient and targeted cargo molecule delivery, such as mRNA delivery.

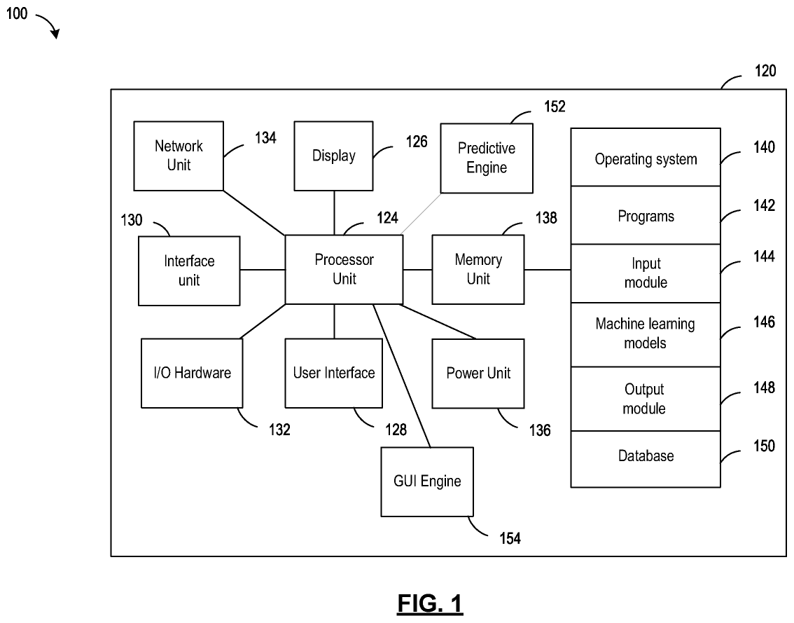

Reference is first made to FIG. 1, showing a block diagram of an example embodiment of system or platform 100 for AI-guided ionizable lipid engineering. The system 100 includes at least one server 120. The server 120 may communicate with one or more user devices (not shown), for example, wirelessly or over the Internet. The system 100 may also be referred to as a machine learning system when used as such.

The user device may be a computing device that is operated by a user. The user device may be, for example, a smartphone, a smartwatch, a tablet computer, a laptop, a virtual reality (VR) device, or an augmented reality (AR) device. The user device may also be, for example, a combination of computing devices that operate together, such as a smartphone and a sensor. The user device may be configured to run an application (e.g., a mobile app) that communicates with other parts of the system 100, such as the server 120.

The server 120 may run on a single computer, including a processor unit 124, a display 126, a user interface 128, an interface unit 130, input/output (I/O) hardware 132, a network unit 134, a power unit 136, and a memory unit (also referred to as “data store”) 138. In other embodiments, the server 120 may have more or less components but generally function in a similar manner. For example, the server 120 may be implemented using more than one computing device.

The processor unit 124 may include a standard processor, such as the Intel Xeon processor, for example. Alternatively, there may be a plurality of processors that are used by the processor unit 124, and these processors may function in parallel and perform certain functions. The display 126 may be, but not limited to, a computer monitor or an LCD display such as that for a tablet device. The user interface 128 may be an Application Programming Interface (API) or a web-based application that is accessible via the network unit 134. The network unit 134 may be a standard network adapter such as an Ethernet or 802.11x adapter.

The processor unit 124 may execute a predictive engine 152 that functions to provide predictions by using machine learning models 146 stored in the memory unit 138. The predictive engine 152 may build a predictive algorithm through machine learning. The training data may include, for example, image data, video data, audio data, and text.

The processor unit 124 can also execute a graphical user interface (GUI) engine 154 that is used to generate various GUIs. The GUI engine 154 provides data according to a certain layout for each user interface and also receives data input or control inputs from a user. The GUI then uses the inputs from the user to change the data that is shown on the current user interface, or changes the operation of the server 120 which may include showing a different user interface.

The memory unit 138 may store the program instructions for an operating system 140, program code 142 for other applications, an input module 144, a plurality of machine learning models 146, an output module 148, and a database 150. The machine learning models 146 may include, but are not limited to, image recognition and categorization algorithms based on deep learning models and other approaches. The database 150 may be, for example, a local database, an external database, a database on the cloud, multiple databases, or a combination thereof.

In at least one embodiment, the machine learning models 146 include a graph neural network. In another embodiment, the machine learning models 146 include Convolutional neural networks (CNNs), which may be designed to recognize spatial patterns in structured data like molecular graphs. In another embodiment, the machine learning models 146 include Recurrent neural networks (RNNs), which may be designed for processing sequences, and which can be adapted for paths or sequence within graph structures.

In some embodiments, the machine learning models 146 may include transformer models, which may be designed to handle sequences and sets, and can be adapted to process graphs by treated nodes and edges as sequential data. In another embodiment, the machine learning models 146 may include autoencoders, which may be useful for unsupervised learning tasks in molecular screening, and learning to encode and decode graph structures efficiently.

In some embodiments, the machine learning models 146 may include one or more of i) Capsule Networks, which may be designed to recognize hierarchical spatial relationships and may potentially handle complex molecular patterns effectively; ii) Message Passing Neural Networks (MPNNs), which may be designed to aggregate information through a message-passing scheme, specifically tailored for graph data; iii) Spectral CNNs, which may use graph Laplacian for capturing global properties of molecular graphs; and iv) Diffusion Neural Networks, which may simulate diffusion processes on graphs, capturing broad structural patterns.

In other embodiments, machine learning models 146 can include other emerging or specialized models that can take molecular graph and/or descriptors as input, and perform regression tasks with molecular data.

The programs 142 comprise program code that, when executed, configures the processor unit 124 to operate in a particular manner to implement various functions and tools for the system 100.

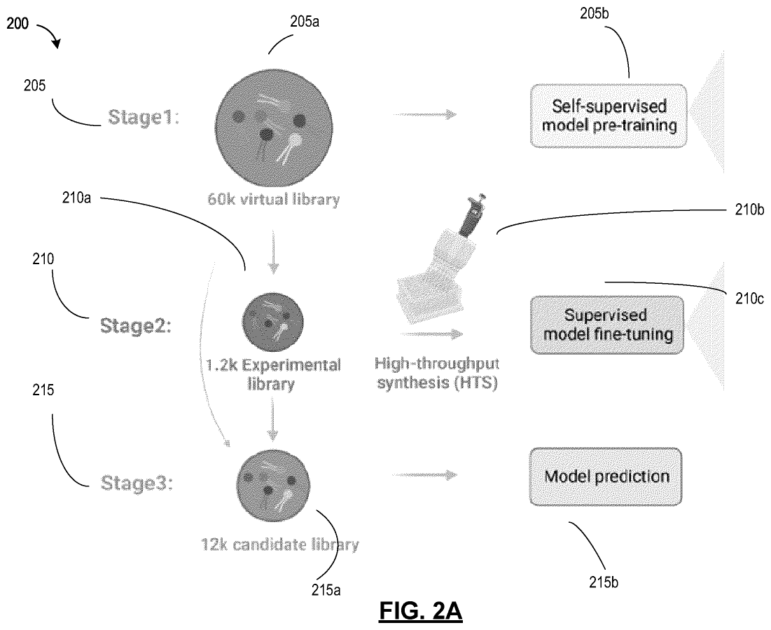

Reference is next made to FIG. 2A, which illustrates a sequence 200 of steps or stages that are performed by the AI-guided ionizable lipid engineering system 100 of FIG. 1.

Sequence 200 includes a first stage 205, which consists of pre-training of the machine learning model used within the system 100. In various embodiments, the first stage 205 also includes construction of a virtual library. In the illustrated embodiment, the pre-training consists of a self-supervised training of the machine learning model. Second stage 210 consists of fine-tuning the pre-trained machine learning model of the first stage. In the illustrated embodiment, the fine-tuning consists of a supervised training of the machine learning model. In various embodiments, the second stage 210 also includes synthesis of an experimental library. The third step 215 consists of a candidate library model prediction, followed by ranking for final candidate selection.

Reference is now made to FIGS. 2A and 2B to discuss the first stage 205. The first stage 205 starts with a machine learning model, such as, one involving a large deep learning neural network, that inherits the structural knowledge learned in millions of small molecules. In one embodiment, the machine learning model includes a graph neural network (GNN) model, which is pretrained on a vast data set of small molecules, such as, over approximately million small molecules. In other embodiments, other machine learning models are used and trained on such small molecules.

In this stage, the machine learning model learns differentiable lipid representations from the large collection of unlabeled lipid molecules. In one embodiment, to enhance the machine learning model with knowledge about ionizable lipid structures, a library of diverse ionizable lipid structures is designed based on 3 component reaction (3-CR) chemistry principles to form a virtual lipid library at step 205a. The virtual lipid library 205a is used to continuously pretrain the model at step 205b.

In the illustrated embodiment, for example guided by combinatorial chemistry methods used for the high through-put construction of lipid molecules, for example based on the 3-CR Ugi reaction or the methods for the preparation of ionizable lipids as defined herein, a virtual lipid library of about 60,000 ionizable lipid structures are designed and inputted at step 205a. In other embodiments, other combinatorial chemistry methods are utilized, and more or less than 60,000 ionizable lipid structures are designed.

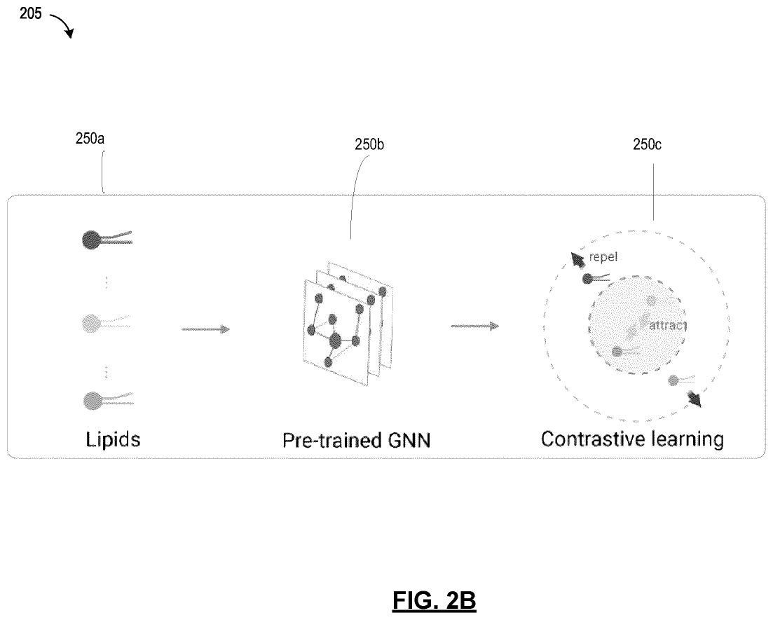

Reference is made to FIG. 3, which illustrates a virtual library design 300 at step 205a. As shown, diverse headgroups 305, connecting groups 310 and two distinct alkyl chains 315, 320 are used to design some 60,000 virtual lipid library for adding to the original chemical library. Examples of diverse headgroups 305 include linear amines, cyclic amines, etc. Examples of diverse connecting groups 310 include ester bonds, amide bonds, etc. Examples of the alkyl chains include alkene tail chains, alkyne tail chains, etc.

Reference is again made to FIG. 2B, which illustrates an example embodiment of steps performed to further train the machine learning model in the first stage. As illustrated in FIG. 2B, the virtual lipid library of 60,000 ionizable lipid structures is used as input to train the machine learning model at step 250a. At step 250b, the pre-trained GNN model, previously trained on millions of small molecules, is used to receive the inputs from step 250a. The GNN model undergoes pretraining by contrastive learning at step 250c. Contrastive learning is a deep learning technique for unsupervised representation learning. The inherited pretraining and continuous pretraining empowers the machine learning model with robust understanding of lipid structure in general, which contributes to the transfection efficiency prediction at later stages.

Reference is next made to the second stage 210 of FIG. 2A. In the second stage, the machine learning model is fine-tuned to enable the machine learning model to predict transfection efficiency of lipids towards a specific cell line. As shown, the second stage includes generation of an experimental library of ionizable lipid structures 210a. The second stage further includes a high-throughput synthesis of the experimental library of ionizable lipids using combinatorial chemistry and assessment of their transfection efficiencies in a target cell line 210b. Next, the second stage includes fine-tuning 210c the machine learning model, using supervised learning, based on in-vitro screening results from step 210b. This provides the advantages of allowing the model to recapitulate the potential transfection ability of molecules.

In one example embodiment, approximately 1,200 ionizable lipids are selected from the virtual library 205a and included in an experimental library. The ionizable lipids from the experimental library are synthesized using an automated high-throughput synthesis platform based on the one-pot Ugi three components reaction (3CR), for example using the method of preparing ionizable lipids as described hereinabove. In some other embodiments, other combinatorial chemistry methods are used to synthesize the ionizable lipids from the experimental library.

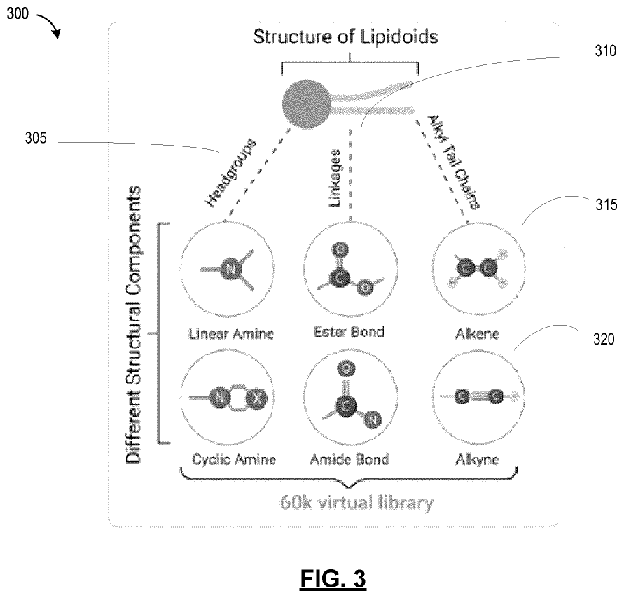

Reference is briefly made to schematic 600 of FIG. 6, illustrating a high-throughput synthesis method for lipidoids. As shown, the method includes multiple components, such as headgroup 605 (for example compounds of Formula A as defined above), a first tailgroup 610 (for example compounds of Formula B as defined above) and a second tailgroup 615 (for example compounds of Formula C as defined above). The method further includes a one-pot synthesis reaction 620 of these multiple components 605, 610, 615, resulting in corresponding modified 3CR-Ugi lipid structures 625. In some embodiments, the one-pot synthesis is the method of preparing ionizable lipids as defined above. Next, the resulting lipid structures 625 undergo a three-dimensional combinatorial screening 630, to generate a synthesized toolbox 635 of about 1,200 lipid structures.

In one example, approximately 1,200 ionizable lipids are synthesized within 24 hours using an automated high-throughput synthesis (HTS) platform. In some examples, using the HTS platform, the LNPs are formulated via a liquid handling robot based on a classical four-composition formulation ratio, containing one of a synthesized ionizable lipids, 2-dioleoyl-sn-glycerol-3-phosphoethanolamine (DOPE), as a helper lipid, cholesterol, and 1,2-dimyristoyl-sn-glycerol-3-phosphoethanolamine-N-[methoxy(polyethyleneglycol)-2000](C14-PEG).

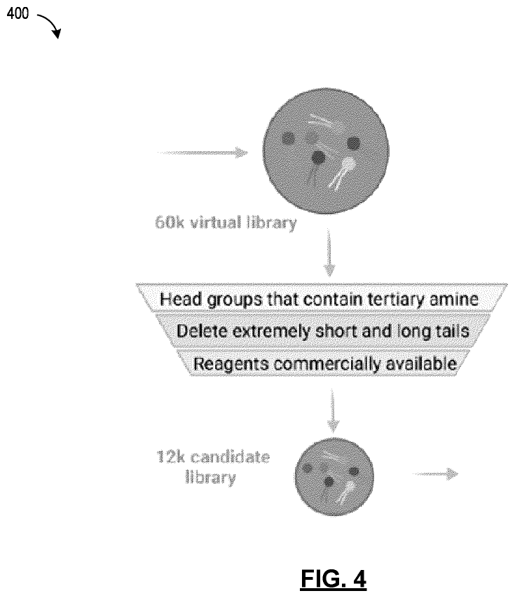

Reference is briefly made to FIG. 7, illustrating a schematic diagram 700 showing the LNP components for mRNA encapsulation in an example embodiment. As shown, the LNP components include the following: cholesterol 705, ionizable lipidoid 710, helper lipid (DOPE) 715, polyethylene glycol (PEG)-phospholipid conjugate (DMG-PEG 2000) 720 and mRNA 725.

In one example, the assessment of the transfection efficiencies of the LNPs from the experimental library involves evaluating the mRNA transfect efficacy of these LNPs in Hela cells. In some cases, the firefly luciferase (Fluc) protein expression activity is measured by encapsulating Fluc mRNA in the LNP.

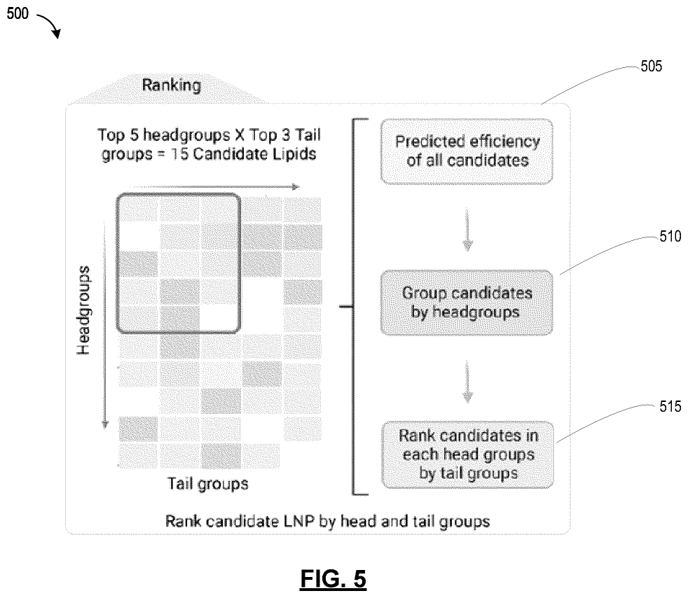

Reference is briefly made to FIG. 8, illustrating a balloon plot 800 of the data used for the fine-tuning of the machine learning model in the second stage. The plot 800 is generated based on Fluc mRNA (mLuc) delivery of 1,200 LNPs from the experimental library and measuring the relative luciferase expression in Hela cells. The plot 800 is based on lipid components, namely amine headgroups 805, aldehyde tails 810 and isocyanide tails 815.

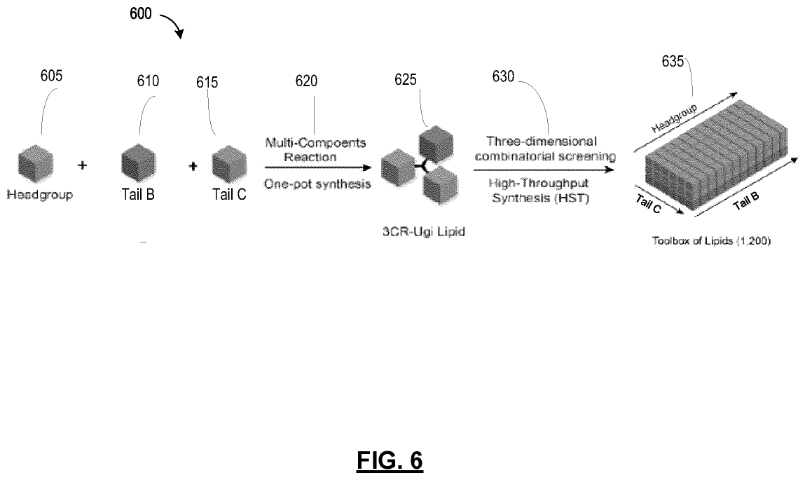

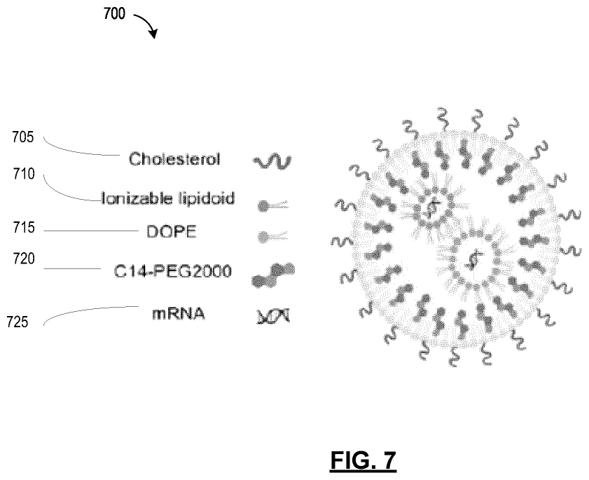

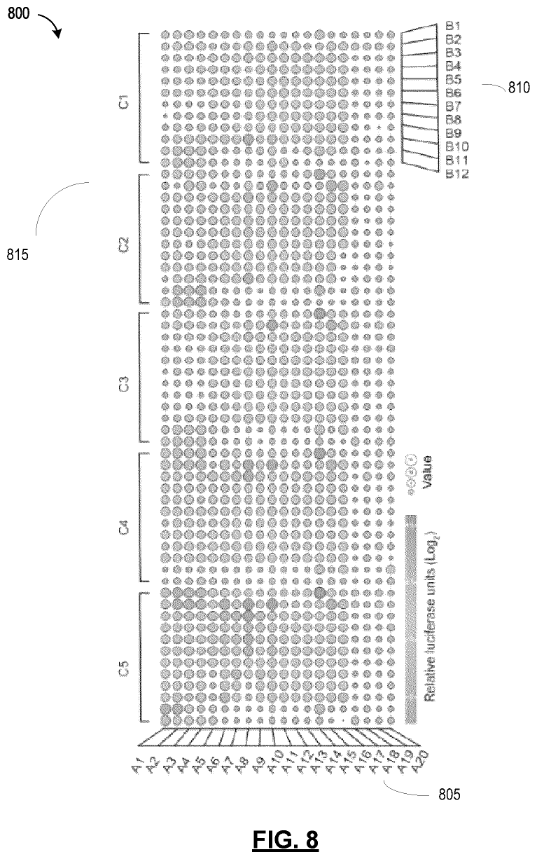

Reference is next made to FIGS. 9-11. FIG. 9 shows a graph 900, illustrating the train loss 905 and validation loss 910 plots. FIG. 10 shows a graph 1000, illustrating the mean square error (mse) score plots (validation rmse plot 1005, test rmse plot 1010). FIG. 11 shows a umap plot 1100 of efficiency of 1,200 LNPs of FIG. 8.

Reference is now made to FIG. 2C to illustrate the steps involved in fine-tuning the machine learning model in the second stage 210. As shown, at step 260a, the lipids from the experimental library and their corresponding transfection efficiency, as measured by synthesizing such lipids and assessing their transfection efficiency in a specific tissue or cell line, are labelled and provided as inputs to the machine learning model. At step 260b, the machine learning model is trained on the inputs from step 260a. In one example, to enhance the model's generalizability and precision, the molecular descriptions using Mordred are extracted and included as inputs for learning efficiency prediction. During the fine-tuning process, the model is trained to minimize the difference between the predicted efficiency and experimental reality. The machine learning model is fine tuned for learning efficiency prediction 260c.

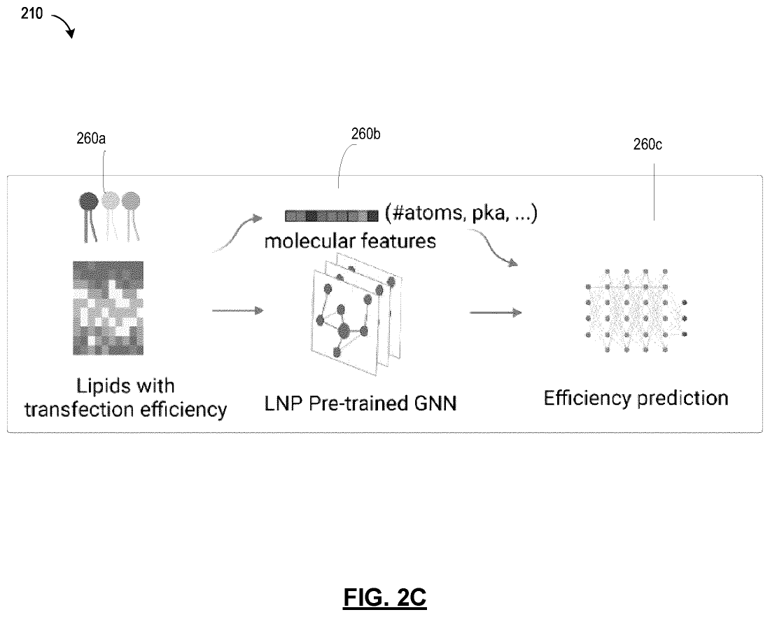

Reference is next made to the third stage 215 of FIG. 2A. In the third stage 215, a candidate library is created based on the virtual library 205a of the first stage 205. In at least one embodiment, as illustrated in FIG. 4, the appropriate candidate structures are selected from the virtual library based on one or more of the following three principles, including (i) removing of cationic lipids which could increase the toxicity of the final LNP delivery system; (ii) removing of ionizable lipids containing alkyl chains which are either too short or too long (such as, for example, less than C6 or greater than C18); and iii) excluding structures containing reagents not commercially available.

In the illustrated embodiment, the candidate library is created by selecting approximately 12,000 appropriate structures from the virtual library of 60,000 structures from the first stage.

In various embodiments, the third stage 215 further includes predicting 215b the efficiencies of the candidate lipid structures in the candidate library. The efficiency prediction is carried out by utilizing the fine-tuned machine learning model of the second stage 210.

Reference is next made to FIG. 2D, which illustrates that once the efficiencies of the candidate lipid structures is determined in the third stage 215, the candidate lipids are ranked 270a based on a ranking scheme. Once ranked, a predetermined number of candidates with a high rank are selected for lipid synthesis and validation 270b.

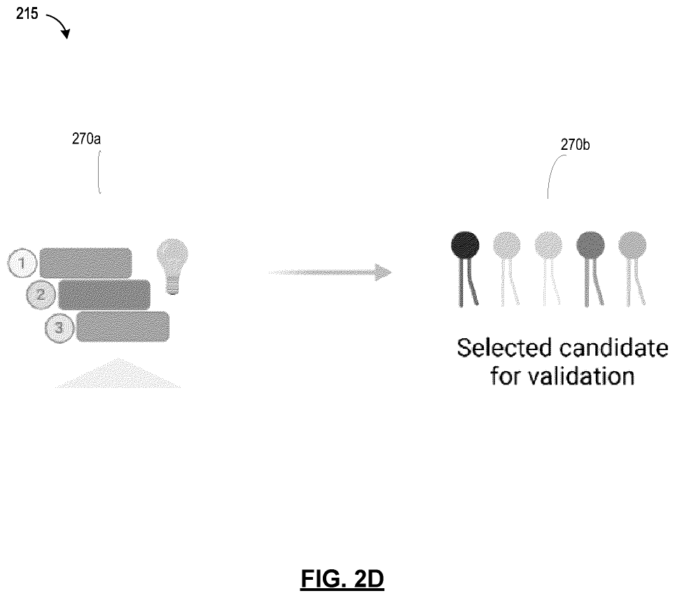

In one embodiment, as illustrated in FIG. 5, the ranking scheme includes ranking candidate lipids by head and tailgroups based on their predicted efficiencies in the third stage. This is because it is observed that the predicted efficiencies exhibit distinct stratification by head and tailgroups, and the structural differences among molecules with the same head and tailgroups are relatively minor.

Accordingly, in this embodiment, once the efficiencies of the candidate lipids are predicted, at step 505, the candidate lipids are first organized by headgroups, at step 510, and subsequently ranked within each headgroup by tailgroups, at step 515, following the same scheme. This provides the advantage of increasing the diversity of the candidates selected for further validation.

In one embodiment, top 15 candidate lipids are selected for further synthesis and validation. In one example, the top 15 candidate lipids are selected by identifying the top 5 headgroups, and selecting the 3 highly ranked tailgroups within each headgroup.

The 15 lead lipid compounds, as discussed above, were rapidly synthesized and evaluated in Hela cells. It was found that the 15 candidates resulted in luciferase protein expression. The in-vivo potential of the 15 lead compounds was also investigated by administering Fluc mRNA encapsulated in LNPs into the muscles of mice.

By this process, the system 100 helped identify ionizable lipid structures and, accordingly, LNP structures, such as, for example, the H278 (I-2)-mediated LNP for intramuscular injection, which demonstrated superior mRNA transfection efficiency compared to MC3 LNP. The H278 (I-2) LNP demonstrated a notably robust bioluminescence signal. After optimizing the KNP formulation of H278(I-2) by using the Design-of-Experiments (DoE), the mRNA transfection efficiency of H278 (I-2) was found to be 2.3 times higher than that of MC3.

Further, the H278 (I-2) LNP was utilized to evaluate mRNA transfection efficiency in vivo by injecting mice intramuscularly. This revealed that the transfection efficiency of the H278(I-2) lipid in muscle was 7.8 times higher than that of the MC3, with no significant difference compared to the ALC-0315.

In order to investigate the potential of H278 (I-2) lipid for vaccination, the efficacy of H278 (I-2) LNP was compared to that of ALC0315 LNP by administering cre-recombinase mRNA LNP to mTmG reporter mouse models. These mice harboured gene mutations in the Gt(ROSA)26Sorlocus, and upon cre-mRNA expression, the mT cassette was excised in the cre-expressing tissue, enabling the expression of the downstream membrane-targeted GFP (mG) cassette. Comparable levels of protein expression were observed at the intramuscular injection site for H278 (I-2) and ALC-0315 LNPs. However, ALC-0315 LNPs showed higher protein expression levels in liver tissue. Quantification of confocal images reveals that H278 (I-2) LNP exhibits 28% lower transfection efficiency in the liver compared to ALC-0315 LNP. Notably, clinical studies have associated ALC-0315-based BNT162b2 mRNA vaccines with autoimmune hepatitis (AIH) following vaccination. Hence, it is anticipated that H278 (I-2) LNP will alleviate the serious potential side effects of hepatitis resulting from the off-target effects of the mRNA-LNP platform.

As previously mentioned, mRNA therapy has the potential to treat diseases that require intracellular protein expression and genome editing. However, effective mRNA delivery requires transfection into the target tissue and cell type. Adeno-associated virus (AAV) vectors have difficulty in transfusing innate immune cells, indicating that it is possible to develop non-viral mRNA delivery vectors for immune cells based on the system 100.

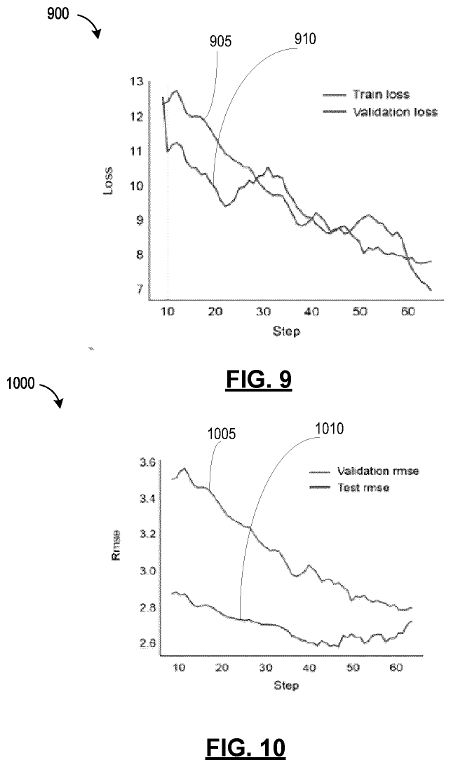

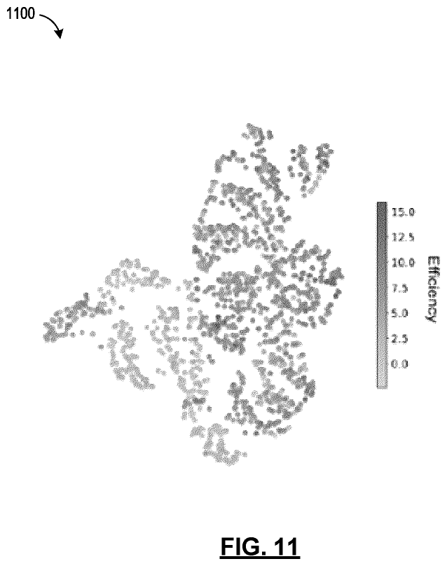

To validate the ability of system 100 to screen ionizable lipids across different cell lines and tissues, system 100 was adapted to the discovery of ionizable lipids that can efficiency transfect immune cells. For this application, the 1,200 lipids from the experimental library in the second stage discussed above (e.g. second stage 210 of FIG. 2) were synthesized and assessed in Raw 264.7 cells to create a macrophage-specific supervised fine-tuning dataset. In this embodiment, LNPs were loaded with Fluc mRNA and tested on both Raw 264.7 and Hela cells to evaluate luciferase protein expression in both these scenarios. The results revealed marked differences in transfection efficiency between these two cell types, with even the same batch of lipids showing disparate outcomes in Hela cells and macrophage cells. Further, fewer lipids performed well in the 1,200 lipid pools in macrophages compared to Hela cells, indicating the higher transfection difficulty of immune cells. FIG. 12 illustrates a plot 1200 of transfection efficiency top 5 headgroups from the data 800 of FIG. 8 in Raw 264.7 cells. FIG. 13 illustrates a plot 1300 of transfection efficiency of top 3 and bottom 2 tailgroups from the data 800 of FIG. 8 in Raw 264.7 cells.

The data from the macrophage application of the synthesized LNPs was then used to train and fine-tune the machine learning model in the second stage. The supervised data trained the machine learning model, such that in the third stage, a new set of 15 potential lipid candidates were identified for better performance with Raw 264.7 cells.

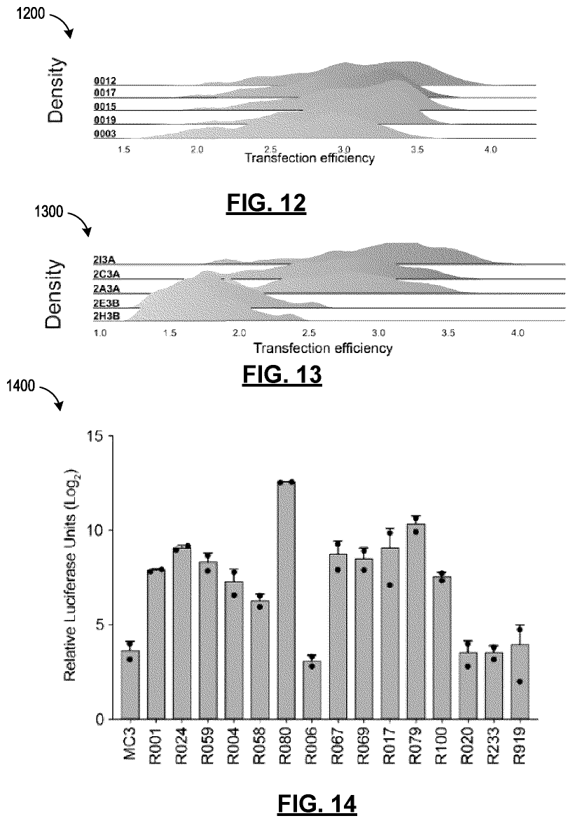

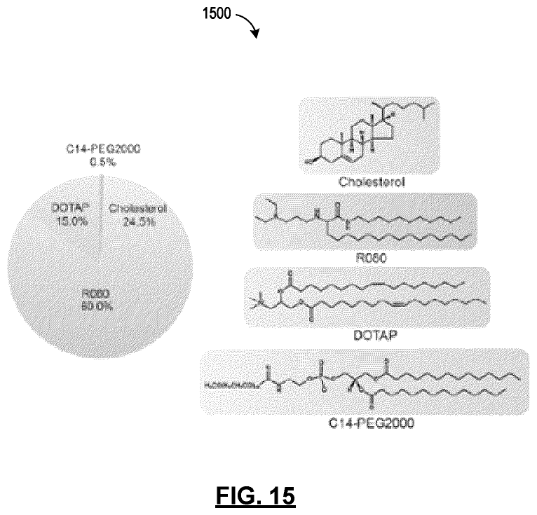

The 15 candidates were synthesized in the lab through the HTS platform and subjected to an initial screen in Raw 264.7 cells, where 11 out of showed superior transfection efficiency compared to MC3. Of these candidates, R080 was identified as the best-performing lipid and was subjected to formulation optimization using DoE. FIG. 14 illustrates a plot 1400 of 15 lipid candidates selected for validation for Raw 264.7 cells. FIG. 15 illustrates a top-performing formulation parameters used in the optimization of R080 (I-3) LNPs in Raw 264.7 cells.

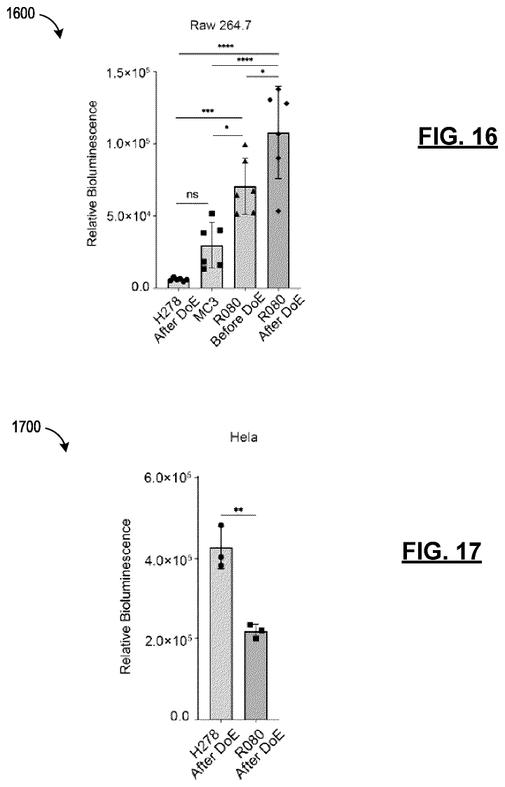

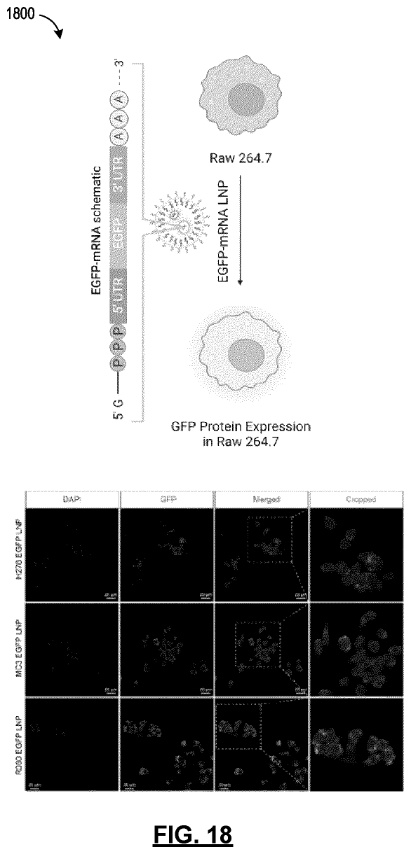

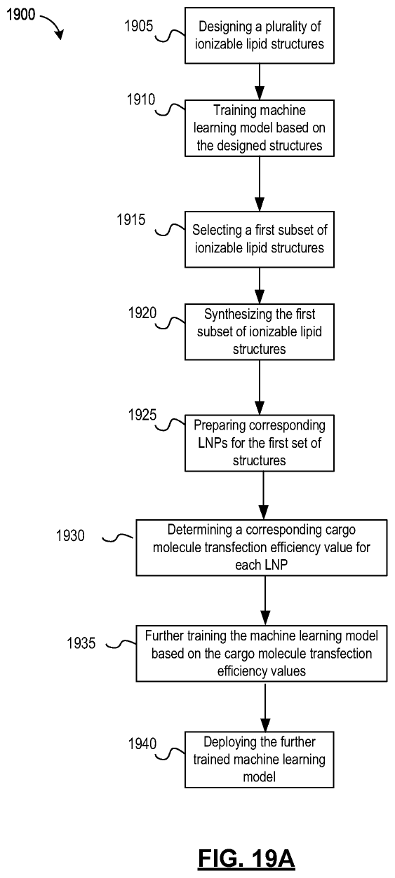

The transfection efficiency of Fluc mRNA was evaluated in Hela cells and Raw 264.7 cells using H278 (I-2) and R080 (I-3), respectively, and formulation optimization. It was noted that H278 (I-2) demonstrated more than a 2-fold increase in transfection efficiency compared to R080 (I-3) in Hela cells. However, R080 (I-3) exhibited significantly higher transfection efficiency in Raw 264.7 cells compared to H278 (I-2) and MC3. FIG. 16 illustrates a comparison of the Fluc-mRNA transfection efficiency of different LNPs in Raw 264.7 cells. (H278 (I-2) LNPs, MC3 LNPs, R080 (I-3) original screen formulation LNPs and optimized formulation LNPs). FIG. 17 illustrates a comparison of the efficacy of AGILE-LNPs (H278 (I-2) LNPs and R080 (I-3) LNPs) in Hela cells. FIG. 18 illustrates confocal images 1800 of R080 (I-3) LNP that was able to successfully deliver GFP mRNA to Raw 264.7 and expressing green fluorescent protein, indicating the potential of this LNP to deliver mRNA drugs.

This demonstrates the benefit of developing a corresponding LNP for a specific type of cell and tissue, rather than one-size-fits-all approach for all targets. System 100 solves this problem for example by i) featuring a large pre-trained deep learning neural network that learns the structural information of lipids, enabling the generation of robust lipid representations; ii) easy adaptability upon vast-scale synthesis and screening of lipids using combinatorial chemistry; and iii) capability of screening a chemical space substantially larger than the synthesized combinatorial library, facilitating the identification of high transfection efficiency lipids for tissue-specific delivery. Further the disclosure herein represents an integration of in-silico and in-vitro screening benefits seamlessly, enabling the exploration of vast chemical spaces beyond the reach of existing experimental methods, thereby significantly accelerating the design and discovery of effective LNPs which offering customization capabilities for various tissues.

Reference is next made to Table 1, providing a list of cell types against which the LNPs can be tested to address specific disease application.

TABLE 1
List of cell types and corresponding applications

| Cells Types | Potential application |
|---|---|
| Hepatocytes | hemophilia |
| Retinal cells | inherited retinal disorders |
| Neurons | Parkinson's disease, Alzheimer's disease, spinal |
| | muscular atrophy (SMA), and amyotrophic lateral |
| | sclerosis (ALS) |
| Cardiomyocytes | cardiovascular diseases |
| Skeletal muscle cells | Duchenne muscular dystrophy |
| Chondrocytes | osteoarthritis and cartilage defects |
| Endothelial cells | vascular disorders |
| T cells | CAR-T immunotherapy |
| dendritic cells | cancer immunotherapy |
| Macrophages | CAR-M immunotherapy |
| Natural Killer cells | CAR-NK immunotherapy |
| Pancreatic beta cells | diabetes |
| Lung epithelial cells | cystic fibrosis and pulmonary |
| | arterial hypertension |
| Hematopoietic stem | blood disorders such as sickle cell disease, |
| cells (HSCs) | thalassemia, and certain types of leukemia |

As discussed above, in order to identify lead LNP candidates for a specific use-case or application, such as hemophilia, Parkinson's disease, diabetes, cardiovascular diseases, etc., the system 100 can be adapted as follows. In the second stage, the lipid structures of the experimental library can be synthesized (such as, by using, a high-throughput synthesis platform) and tested for transfection efficiency in the corresponding cell type identified in Table 1. For example, if candidate LNPs for CAR-T immunotherapy are to be identified, the second stage of FIG. 2 can include conducting wet lab experiments on immune cells, such as T cells, and transfection efficiency data can accordingly be collected. Such experimental data is then used to fine-tune the machine learning model.

Next, in the third stage of FIG. 3, the 12,000 candidates from the candidate library can be provided as input to the fine-tuned machine learning model, to generate a predicted efficiency of such candidates. The candidates and the predicted efficiencies can be ranked according to the ranking schema discussed herein (such as, for example, the ranking schema illustrated in FIG. 5) to generate the lead candidates for the CAR-T immunotherapy application.

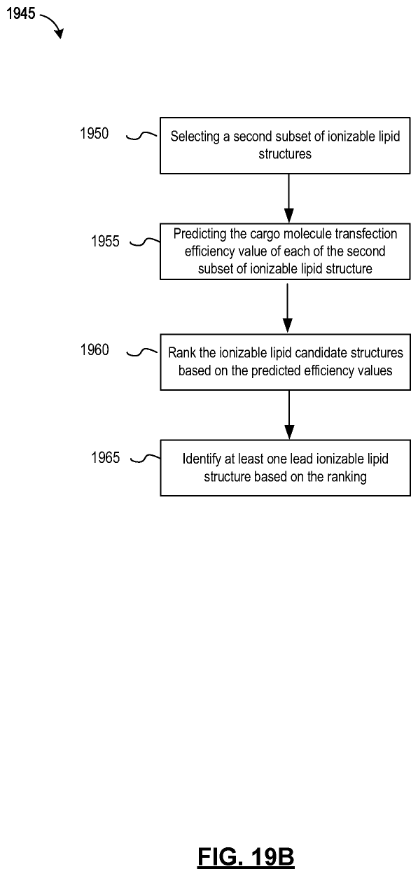

Reference is next made to FIG. 19A illustrating a AI-based method 1900 for ionizable lipid engineering for lipid nanoparticle (LNP)-based cargo molecule delivery, according to an example embodiment. The method of FIG. 19A can be carried out on any system analogous to system 100 of FIG. 1.

As discussed herein, the platform 100 and the methods for AI based ionizable lipid engineering combine deep learning methodologies with high-throughput combinatorial lipid synthesis chemistry to enhance the discovery of new ionizable lipid structures essential for LNP-based delivery of cargo molecules in target cells. In various embodiments disclosed herein, the system 100 comprises deep learning algorithms including a graph encoder and a molecular descriptor encoder to effectively discern the unique characteristics and chemical properties of ionizable lipid molecular structures. In various embodiments, molecular structures of ionizable lipids are represented as graphs where nodes represent the atoms (e.g., as on the periodic table) and edges represent the chemical bons (e.g., single, double, triple, or aromatic bonds, etc.). For each molecule, a simplified molecular-input line-entry system (SMILES) representation can be converted into a molecular graph, which is then input to the neural network model in the platform 100. This representation provides the advantage of capturing the topological structure and properties of a molecule effectively.

The method 100 comprises designing, at step 1905, a plurality of ionizable lipid structures based on a combination of a plurality of lipid headgroups, a plurality of first tailgroup and a plurality of second tailgroup. In some embodiments, this step comprises designing the ionizable lipid structures by selecting a variety of lipid headgroups, first and second tail groups based on suitability for preparation of the ionizable lipid using a high throughput synthesis method, for example using combinatorial chemistry. In some embodiments, the high throughput synthesis method comprises the method of preparing the compounds of Formula I as described herein, i.e. by reacting a compound of Formula A with a compound of Formula B and a compound of Formula C in the presence of a catalyst in a one pot reaction. Therefore, in some embodiments, the selecting of the lipid headgroups, first and second tail groups is based on availability of starting reagents (for example compounds of Formulae A, B and C) that are suitable for reacting to obtain a ionizable lipid structure.

Next, at step 1910, the method comprises training a machine learning model based on the plurality of ionizable lipid structures. This enables the machine learning model to establish a graph encoder with an extensive understanding of lipid structures. In one embodiment, approximately 60,000 lipids are designed at step 1905, and the machine learning model is trained at step 1910 on the 60,000 lipids in a library. The machine learning model may be trained using contrastive learning to optimize the model's performance within the lipid domain.

In some embodiments, the method comprises pre-training the machine learning model, prior to step 1910, to learn generalizable lipid representation that can benefit the downstream prediction. For instance, before the lipid-specific pre-training at step 1910, the machine learning model may be initialized by model parameters with the general pre-trained model, which may be trained on approximately over 10 million distinct small molecules. This may provide the benefit of a warm start to the machine learning model that has already been trained to capture molecular structures.

Referring again to step 1910, in one embodiment, the machine learning model is trained to learn ionizable lipid representation through contrastive positive data pairs against negative pairs. For example, the model is trained to minimize the following loss equation (1):

$${L}_{i , j} = - log \frac{exp ( \frac{sim ( {z}_{i} , {z}_{j} )}{τ} )}{{\sum }_{k = 1}^{2 N} 𝕝 \{ k \neq i \} exp ( \frac{sim ( {z}_{i} , {z}_{k} )}{τ} )} , \qquad Eq . ( 1 )$$

$$sim ( {z}_{i} , {z}_{j} ) = \frac{{z}_{i} {z}_{j}}{{‖ {z}_{i} ‖}_{2} {‖ {z}_{j} ‖}_{2}} ,$$

where zᵢ and zⱼ are the learned lipid representation vectors extracted from a positive data pair, N is the batch size, and T is the temperature parameter, which may be set manually.

In one embodiment, at step 1910, the lipid representation used in the machine learning model only contains the graph structure representation. In such embodiment, to construct the positive data pair, each input lipid molecule graph is transformed into two different but correlated molecule graphs using graph augmentation. The molecule graphs augmented from the same molecule are denoted as a positive pair, and those from different molecules are denoted as negative pairs within each batch. During training, the model learns to maximize the agreement of positive pairs while minimizing the agreement of negative ones.

In some embodiments, one or more of the following data augmentation strategies are adopted: i) Atom masking: Within the lipid molecular graph, atoms are randomly masked according to a specified ratio. This process enables the model to assimilate chemical information, such as atom types and corresponding chemical bond varieties within lipid molecules. ii) Bond deletion: Chemical bonds interconnecting atoms are randomly removed in accordance with a designated ratio. As the formation and dissociation of chemical bonds dictate the properties of ionizable lipid molecules during chemical reactions, bond deletion facilitates the model's learning of correlations between ionizable lipid molecule involvement in various reactions.

At step 1915, the method comprises selecting a first subset of ionizable lipid structures from the plurality of ionizable lipid structures and designating the first subset of ionizable lipid structures as experimental ionizable lipid structures.

Next, step 1920 comprises synthesizing, using a high throughput synthetic method, each of the experimental ionizable lipid structures, according to the embodiments disclosed herein. Step 1925 comprises preparing a first set of separate lipid nanoparticles (LNPs), where each LNP comprises a unique experimental ionizable lipid structure and the cargo molecule.

Step 1930 comprises determining a corresponding cargo molecule transfection efficiency value of each of the prepared LNPs in a target cell. As discussed herein, the target cell can be any one or more of the following cells: Hela cells, T-cells, stem cells, lung epithelial cells, pancreatic beta cells, natural killer cells, macrophages, dendritic cells, endothelial cells, chondrocytes, skeletal muscle cells, cardiomyocytes, neurons, retinal cells and hepatocytes.

Step 1935 comprises further training the machine learning model based on the prepared LNPs and the corresponding cargo molecule transfection efficiency values. This step provides the benefit of helping the model understand the correlation between lipid molecular properties and cargo molecule transfection efficiencies.

In various embodiments, during the fine-tuning step 1935, a molecular descriptor encoder is used along with the graph encoder to represent the lipid molecule. In some cases, the property descriptor input is generated by executing a series of preprocessing procedures aiming to isolate pertinent features of the lipid molecules.

In one example, the preprocessing procedure included eliminating descriptors with a standard deviation of zero, followed by the selection of descriptors exhibiting correlation with the experimentally determined transfection efficiency in both HeLa and Raw 264.7 cells (score of R2>0.006), resulting in the identification of 813 salient descriptors. Subsequently, log transformation was applied to the descriptors possessing extensive data ranges, with normalization conducted accordingly.

The following table 2 shows an example of 50 molecular descriptors and their respective correlation values in both Hela and Raw 264.7 cells, which may be used as data for fine-tuning the machine learning model at step 1935.

In one embodiment, the machine learning model was fine-tuned at step 1935 by performing regression on the cargo molecule transfection efficiency values. In one example, the mean squared loss between the predicted and ground-trust potency is used to optimize the model parameters using equation (2):

$${L}_{m s e} = \frac{1}{n} {\sum }_{i = 1}^{n} {( P r e d ( {z}_{i} ) - {y}_{i} )}^{2} , \qquad Eq . ( 2 )$$

where Pred(⋅) denotes the fully connected layers that perform the cargo molecule transfection efficiency prediction, and yᵢ is the actual transfection efficiency recorded in vitro.

In one example, a scaffold-based 80%-10%-10% train-valid-test split is performed on the experimental library of step 1915, and at step 1935, the machine learning model is fine-tuned on the training set only and the performance on the validation set is evaluated using root mean squared error (RMSE) and Pearson correlation with the ground truth transfection efficiency values.

In some cases, to enhance the machine learning model's robustness and generalizability, the step 1935 is carried out multiple times, such as, for example, ten times, from which the top few models, such as five models, are selected based on RMSE and Pearson correlation performance on the testing data used in step 1935.

Next, step 1940 comprises deploying the further trained machine learning model to identify at least one lead ionizable lipid structure.

In on example, five models resulting from step 1935 are employed for ensemble prediction on candidate lipid structures. In this example, the cargo molecule transfection efficiency predictions are generated from each model to calculate the average and standard deviation of the five predicted values from each candidate molecule. The mean predicted values are then subtracted from the standard deviation, and the resulting predicted score is used to rank the candidates to determine one or more lead ionizable lipid structures.

Reference is next made to FIG. 19B, which illustrates a method 1945 for deploying the further trained machine learning model to identify one or more lead ionizable lipid structures, according to an example embodiment.

Method 1945 comprises selecting, at step 1950, a second subset of ionizable lipid structures from the plurality of ionizable lipid structures and designating the second subset of ionizable lipid structures as ionizable lipid candidate structures. Next, step 1955 comprises predicting the cargo molecule transfection efficiency values of each of the ionizable lipid candidate structures based on the further trained machine learning model. The method further comprises ranking, at step 1960, the ionizable lipid candidate structures based on the corresponding predicted cargo molecule transfection efficiency values in accordance with a ranking schema. Next, the step 1965 comprises identifying the at least one lead ionizable lipid structure based on the ranking of the ionizable lipid candidate structures for in-vitro and/or in-vivo validation.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

### EXAMPLES

The following non-limiting examples are illustrative of the present application.

### Materials and Lipid Library Synthesis.

All materials were prepared and processed without nucleases throughout the synthesis and formulation steps. Firefly luciferase mRNA (mFLuc, Translate), Cre recombinase mRNA (TriLink BioTechnologies), Full-length ovalbumin mRNA (mOVA, TriLink BioTechnologies) and EGFP-mRNA (TriLink BioTechnologies) were directly purchased from vendors. PrestoBlue™ Cell Viability Reagent and Quant-It™ RiboGreen RNA Assay Kit were purchased from Thermo Fisher Scientific Inc. All mRNAs were stored at −80° C. and were allowed to thaw on ice before use. DLin-MC3-DMA and ALC-0315 were purchased from Echelon Biosciences. Amines and other starting materials were obtained from Sigma-Aldrich and TCI America. The tails were purified through flash column chromatography, and their final structures were confirmed using ¹H 400 MHz NMR spectrometry with CDCl₃ and tetramethylsilane (TMS) as a standard at UHN Nuclear Magnetic Resonance Core Facility. To synthesize compounds in each well of a 96-well plate with glass inserts 10 μL of a 350 μM stock solution containing amines and tails were placed into each well of a 96-well plate with glass inserts and pre-stirred overnight. This stock solution was made by mixing the amines and tail components in a 1:1:1 ratio and dissolving them in a 2:1 mixture of methanol with 0.2 eqv. catalyst phenyl hypophosphoric acid (H₃PO₄). The plates were covered and placed on a shaker to stir overnight, with conversion yields typically over 70%. The lipids were formulated into LNP in the same reaction plates. Some lipids were purified through flash column chromatography, and their final structures were confirmed using ¹H 400 MHz NMR spectrometry with CDCl₃ and tetramethylsilane (TMS) as a standard at UHN Nuclear Magnetic Resonance Core Facility. To further analyze these materials, high-resolution mass spectra using an LC-Mass spectrophotometer at the Centre for Pharmaceutical Oncology of the University of Toronto were obtained.

### LNP Synthesis and Formulation for High Throughput Screening.

To conduct high-throughput screening, an organic phase was created by dissolving a mixture of cationic lipid, DOPE (Avanti), cholesterol (Chol, Sigma-Aldrich), and C14-PEG 2000 (Avanti) in ethanol at a predetermined molar ratio. The aqueous phase was prepared using firefly luciferase mRNA (mLuc, Translate), Cre recombinase mRNA (TriLink BioTechnologies) or EGFP-mRNA (TriLink BioTechnologies) in 10 mM sodium citrate buffer (pH 4.0, Fisher). All mRNAs were stored at −80° C. and were allowed to thaw on ice before use. During the high-throughput screening phase, LNPs were synthesized by mixing an aqueous phase containing the mRNA with an ethanol phase containing the lipids using an OT-2 pipetting robot. The aqueous phase was prepared in a 10 mM citrate buffer with the corresponding mRNA. The ethanol phase was prepared by solubilizing a mixture of ionizable lipid, helper phospholipid (DOTAP or DOPE, cholesterol, and C14-PEG 2000 at pre-determined molar ratios with an ionizable lipid/mRNA weight ratio of 10 to 1.

### LNP Synthesis and Formulation for In Vitro and Vivo Tests.

For other in vitro and in vivo tests, all materials were prepared and processed without nucleases throughout the synthesis and formulation steps. DLin-MC3-DMA and ALC-0315 were purchased from Echelon Biosciences. MC3-LNP was prepared at the molar ratio of 50:10:38.5:1.5 (DLin-MC3-DMA:DSPC:cholesterol:DMG-PEG2000) and ALC0315-LNP was prepared at the molar ratio of 46.3:9.4:42.7:1.6 (ALC-0315:DSPC:cholesterol:ALC0159 [Echelon Biosciences]). The optimal formulations of H278 and R080 LNPs for the subsequent experiments were determined by the LNP formulation optimization method. Except for the high-throughput screening, the aqueous and ethanol phases were rapidly mixed by pipette at a 3:1 volumetric ratio. Post incubation for 15 min in a 4° C. fridge.

### LNP Formulation Optimization.

The statistical software JMP 16 (SAS Institute) analyzed the experimental data. In this Design of experiments (DoE) approach, the four-factor Box-Behnken design was suitable for second-order models comprising 17 preparation runs. The design was cited as a common experimental design for screening crucial factors. In this design, all factors (lipid/mRNA weight ratio, ionizable lipid molar ratio, helper lipid molar ratio, and PEG molar ratio) have the low, center, and high levels.

### In Vitro High Throughput Screening.

The lipid library, which was not purified, was directly combined with ethanol and the aqueous solution of mLuc. For in vitro transfection, the lipid-mRNA mixture, containing 0.1 μg of mRNA, was added to pre-seeded Hela and Raw264.7 cells in 96-well plates. Following overnight incubation, the transfection efficiency of mLuc was measured using the One-Glo Luciferase Assay System (Promega), following the manufacturer's instructions. The luminescence was quantified using the Cytation imaging reader (BioTek). Finally, the resulting bioluminescence values are assigned to each SMILE string.

In Vivo Luciferase mRNA for Bioluminescence.

At 6 h after the intramuscular (i.m.) administration of the mRNA LNPs, mice were injected intraperitoneally (i.p.) with 0.2 ml d-luciferin (10 mg/ml in PBS). The mice were anesthetized in a ventilated anesthesia chamber with 1.5% isofluorane in oxygen and imaged 10 min after the injection with an in vivo imaging system (IVIS, PerkinElmer). Luminescence was quantified using the Living Image software (PerkinElmer). C57BL/6 mice (4-8 weeks) were purchased from the Jackson Laboratories.

### ROAS^{mT/mG}Cre Reporter Mice Transfection Analysis

For gene-recombinant Cre mRNA delivery, LNPs co-formulated with Cre mRNA were i.m. injected into ROSA^{mT/mG} Cre reporter mice (The Jackson Laboratory). After 7 d, mice were killed, and major organs were collected and imaged using an IVIS imaging system (PerkinElmer). For direct fluorescence imaging, organs and muscle tissues were fixed in 4% buffered paraformaldehyde overnight at 4° C., then equilibrated in 30% sucrose overnight at 4° C. before freezing in OCT. Three nonconsecutive sections from each organ sample were mounted with DAPI to visualize nuclei and imaged for DAPI, tdTomato, and GFP. sectioned into 10 μm depth, and further imaged using a Fluorescence microscope (Zeiss AXIO Observer 7 Inverted LED Fluorescence Motorized Microscope).

### Statistical Analysis

The data were subjected to statistical analyses using GraphPad Prism 9 (GraphPad Software). A two-tailed unpaired Student's t-test was conducted to assess the significance of the comparisons as indicated. Data are expressed as mean±s.d. P values<0.05 (*), P<0.01 (**), P<0.001 (***) and P<0.0001 (****) were statistically significant.

Lipid synthesis and Characterization

### General Synthesis Route of Lipid Tail A

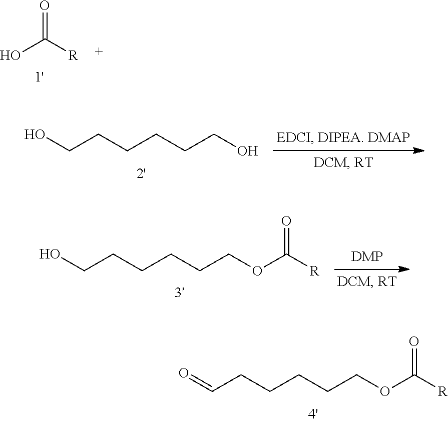

### Acids (1′) Used:

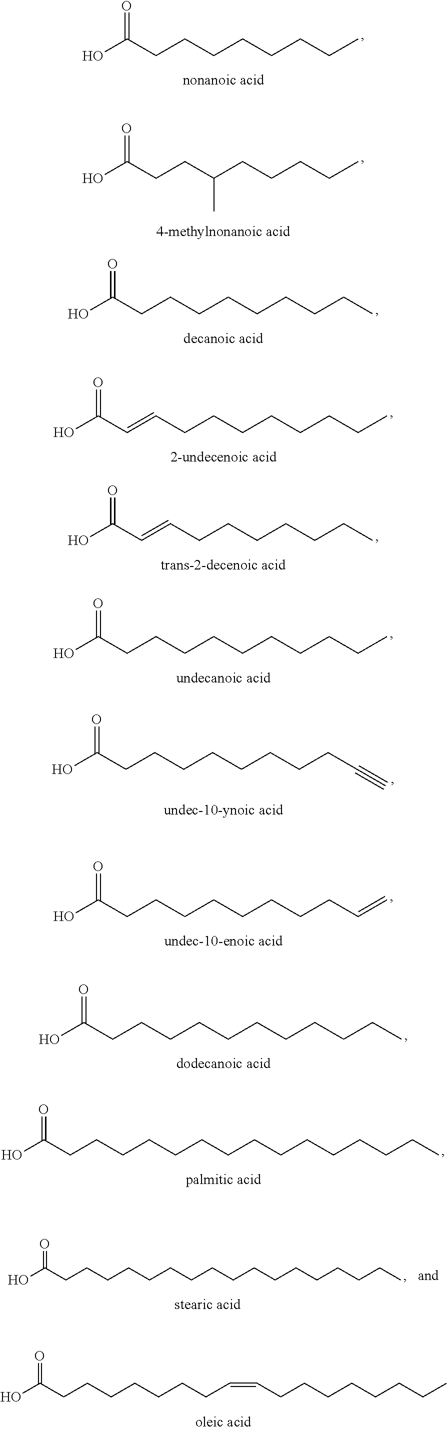

Acid (1′, 17.7 mmol), N-(3-dimethyl aminopropyl)-N′-ethyl carbodiimide hydrochloride (EDC HCl, 26.6 mmol), hexane-1,6-diol (2′, 88.5 mmol), 4-(dimethylamino) pyridine (DMAP, 8.9 mmol) and N, N-diisopropylethylamine (DIPEA, 35.4 mmol) were dissolved in dichloromethane (200 ml). The reaction was stirred at room temperature under nitrogen for 18 h, then washed with a saturated aqueous sodium bicarbonate solution. The organic layer was separated, washed with brine, dried over Na₂SO₄, filtered, and the filtrate was evaporated under a vacuum. The residue was purified by silica gel chromatography (0-50% ethyl acetate in hexanes) to give compound 3′. 3′ (15.2 mmol) was dissolved in dichloromethane (DCM) 300 ml followed by Dess-Martin Periodinane (9.6 g, 22.8 mmol). The reaction was stirred under nitrogen at room temperature for 2 h. After confirmation of reaction completion by thin layer chromatography, sodium thiosulfate pentahydrate (50% w/v, 200 ml) was added to the reaction and left to stir for additional 15 mins. The organic layer was thereafter separated, washed with brine, dried over Na₂SO₄, filtered, and the filtrate was evaporated under a vacuum. The residue was purified by silica gel chromatography (0-50% ethyl acetate in hexanes) to give compound 4′.

### Synthesis of Tail 3′

### Synthesis of Tail 6-hydroxyhexyl nonanoate

Followed the synthesis method above. ¹H NMR (400 MHz, CDCl₃) δ 4.02 (t, J=6.7 Hz, 2H), 3.59 (t, J=6.6 Hz, 2H), 2.25 (t, J=7.6 Hz, 2H), 1.67-1.47 (m, 6H), 1.35 (p, J=4.0 Hz, 4H), 1.30-1.13 (m, 10H), 0.91-0.76 (m, 3H).

### Synthesis of Tail 6-hydroxyhexyl 4-methylnonanoate

Followed the synthesis method above. ¹H NMR (400 MHz, CDCl₃) δ 4.02 (dd, J=7.2, 6.3 Hz, 2H), 3.59 (t, J=6.6 Hz, 2H), 2.36-2.12 (m, 2H), 1.64-1.47 (m, 5H), 1.40-1.15 (m, 13H), 1.10-0.71 (m, 6H).

### Synthesis of Tail 6-hydroxyhexyl decanoate

Followed the synthesis method above. ¹H NMR (400 MHz, CDCl₃) δ 4.02 (t, J=6.7 Hz, 2H), 3.58 (t, J=6.6 Hz, 2H), 2.24 (t, J=7.6 Hz, 2H), 1.73-1.46 (m, 6H), 1.39-1.31 (m, 4H), 1.26-1.17 (m, 11H), 0.85-0.80 (m, 3H).

### Synthesis of Tail 6-hydroxyhexyl undec-2-enoate

Followed the synthesis method above. ¹H NMR (400 MHz, CDCl₃) δ 6.92 (dt, J=15.6, 7.0 Hz, 1H), 5.87-5.41 (m, 1H), 4.05 (dt, J=19.2, 6.7 Hz, 2H), 3.58 (t, J=6.6 Hz, 2H), 2.15 (qd, J=7.1, 1.6 Hz, 2H), 1.72-1.46 (m, 4H), 1.45-1.31 (m, 6H), 1.25-1.21 (m, 6H), 0.94-0.69 (m, 3H).

### Synthesis of Tail 6-hydroxyhexyl (E)-dec-2-enoate

Followed the synthesis method above. ¹H NMR (400 MHz, CDCl₃) δ 6.91 (dt, J=15.7, 7.0 Hz, 1H), 5.76 (dt, J=15.6, 1.6 Hz, 1H), 4.04 (dt, J=23.5, 6.7 Hz, 2H), 3.58 (t, J=6.6 Hz, 2H), 2.27-2.06 (m, 2H), 1.76-1.46 (m, 5H), 1.45-1.30 (m, 6H), 1.24-1.19 (m, 6H), 0.89-0.76 (m, 3H).

### Synthesis of Tail 6-hydroxyhexyl undecanoate

Followed the synthesis method above. ¹H NMR (400 MHz, CDCl₃) δ 4.02 (t, J=6.7 Hz, 2H), 3.59 (t, J=6.6 Hz, 2H), 2.25 (t, J=7.6 Hz, 2H), 1.70-1.45 (m, 6H), 1.41-1.31 (m, 4H), 1.30-1.13 (m, 14H), 0.93-0.71 (m, 3H).

### Synthesis of Tail 6-hydroxyhexyl undec-10-ynoate

Followed the synthesis method above. ¹H NMR (400 MHz, CDCl₃) δ 4.02 (t, J=6.7 Hz, 2H), 3.59 (t, J=6.6 Hz, 2H), 2.25 (t, J=7.5 Hz, 2H), 2.13 (td, J=7.1, 2.7 Hz, 2H), 1.90 (t, J=2.6 Hz, 1H), 1.67-1.42 (m, 8H), 1.40-1.15 (m, 12H).

### Synthesis of Tail 6-hydroxyphenyl undec-10-enoate

Followed the synthesis method above. ¹H NMR (400 MHz, CDCl₃) δ 5.74 (ddt, J=16.9, 10.2, 6.7 Hz, 1H), 5.04-4.77 (m, 2H), 4.00 (t, J=6.7 Hz, 2H), 3.56 (t, J=6.6 Hz, 2H), 2.23 (t, J=7.5 Hz, 2H), 1.97 (dt, J=8.0, 6.7 Hz, 2H), 1.70-1.43 (m, 6H), 1.41-0.97 (m, 14H).

### Synthesis of Tail 6-hydroxyhexyl dodecanoate

Followed the synthesis method above. ¹H NMR (400 MHz, CDCl₃) δ 4.02 (t, J=6.7 Hz, 2H), 3.58 (t, J=6.6 Hz, 2H), 2.25 (t, J=7.6 Hz, 2H), 1.65-1.46 (m, 6H), 1.43-1.06 (m, 20H), 1.02-0.69 (m, 3H).

### Synthesis of Tail 6-hydroxyhexyl palmitate

Followed the synthesis method above. ¹H NMR (400 MHz, CDCl₃) δ 4.06 (t, J=6.7 Hz, 2H), 3.64 (t, J=6.5 Hz, 2H), 2.28 (t, J=7.6 Hz, 2H), 1.60 (dddd, J=14.5, 12.9, 6.8, 3.2 Hz, 6H), 1.38 (p, J=3.3 Hz, 4H), 1.25 (s, 22H), 0.97-0.76 (m, 3H).

### Synthesis of Tail 6-hydroxyhexyl stearate

Followed the synthesis method above. ¹H NMR (400 MHz, CDCl₃) δ 4.04 (t, J=6.7 Hz, 2H), 3.62 (t, J=6.6 Hz, 2H), 2.27 (t, J=7.6 Hz, 2H), 1.59 (dddd, J=15.1, 9.8, 6.7, 3.7 Hz, 7H), 1.37 (p, J=3.8, 3.3 Hz, 4H), 1.23 (s, 28H), 0.90-0.77 (m, 3H).

### Synthesis of Tail 6-hydroxyhexyl oleate

Follow the synthesis method above. ¹H NMR (400 MHz, CDCl₃) δ 5.46-5.21 (m, 2H), 4.04 (t, J=6.7 Hz, 2H), 3.62 (t, J=6.6 Hz, 2H), 2.27 (t, J=7.6 Hz, 2H), 2.06-1.85 (m, 4H), 1.63-1.53 (m, 6H), 1.41-1.34 (m, 4H), 1.33-1.16 (m, 20H), 0.97-0.79 (m, 3H).

### Synthesis of Tail 4′

### Synthesis of Tail 6-oxohexyl nonanoate

Followed the synthesis method above. ¹H NMR (400 MHz, CDCl₃) δ 9.76 (t, J=1.7 Hz, 1H), 4.05 (t, J=6.6 Hz, 2H), 2.57-2.07 (m, 4H), 1.75-1.49 (m, 6H), 1.46-1.33 (m, 2H), 1.32-1.18 (m, 11H), 0.91-0.80 (m, 3H).

### Synthesis of Tail 6-oxohexyl 4-methylnonanoate

Followed the synthesis method above. ¹H NMR (400 MHz, CDCl₃) δ 9.76 (t, J=1.7 Hz, 1H), 4.05 (t, J=6.6 Hz, 2H), 2.48-2.26 (m, 4H), 1.69-1.54 (m, 6H), 1.40 (tdd, J=8.2, 6.5, 4.5 Hz, 4H), 1.29-1.21 (m, 8H), 0.86 (td, J=6.6, 1.3 Hz, 6H).

### Synthesis of Tail 6-oxohexyl decanoate

Followed the synthesis method above. ¹H NMR (400 MHz, CDCl₃) δ 9.74 (t, J=1.7 Hz, 1H), 4.04 (t, J=6.6 Hz, 2H), 2.55-2.14 (m, 4H), 1.62 (ddq, J=14.3, 9.9, 7.3 Hz, 6H), 1.43-1.13 (m, 14H), 0.92-0.78 (m, 3H).

### Synthesis of Tail 6-oxohexyl undec-2-enoate

Followed the synthesis method above. ¹H NMR (400 MHz, CDCl₃) δ 9.76 (t, J=1.7 Hz, 1H), 6.95 (dt, J=15.6, 7.0 Hz, 1H), 5.79 (dt, J=15.6, 1.6 Hz, 1H), 4.12 (t, J=6.6 Hz, 2H), 2.48-2.25 (m, 3H), 2.18 (qd, J=7.1, 1.6 Hz, 2H), 1.68-1.62 (m, 4H), 1.45-1.35 (m, 5H), 1.26-1.23 (m, 6H), 0.86 (d, J=7.1 Hz, 3H).

### Synthesis of Tail 6-oxohexyl (E)-dec-2-enoate

Followed the synthesis method above. ¹H NMR (400 MHz, CDCl₃) δ 9.82-9.66 (m, 1H), 6.93 (dt, J=15.6, 6.9 Hz, 1H), 5.77 (dt, J=15.6, 1.8 Hz, 1H), 4.09 (t, J=6.6 Hz, 2H), 2.43 (td, J=7.3, 1.7 Hz, 2H), 2.16 (qd, J=7.3, 1.6 Hz, 2H), 1.71-1.61 (m, 4H), 1.46-1.35 (m, 4H), 1.26-1.18 (m, 6H), 0.84 (t, J=6.7 Hz, 3H).

### Synthesis of Tail 6-oxohexyl undecanoate

Followed the synthesis method above. ¹H NMR (400 MHz, CDCl₃) δ 9.74 (t, J=1.7 Hz, 1H), 4.04 (t, J=6.6 Hz, 2H), 2.43 (td, J=7.3, 1.7 Hz, 2H), 2.26 (t, J=7.6 Hz, 2H), 1.61 (ddt, J=16.2, 9.8, 7.3 Hz, 6H), 1.42-1.34 (m, 2H), 1.24 (d, J=9.2 Hz, 14H), 0.87-0.82 (m, 3H).

### Synthesis of Tail 6-oxohexyl undec-10-ynoate

Followed the synthesis method above. ¹H NMR (400 MHz, CDCl₃) δ 4.02 (t, J=6.6 Hz, 2H), 2.41 (td, J=7.3, 1.7 Hz, 1H), 2.32 (t, J=7.5 Hz, 1H), 2.24 (t, J=7.5 Hz, 2H), 2.13 (td, J=7.1, 2.7 Hz, 2H), 1.90 (t, J=2.7 Hz, 1H), 1.65-1.55 (m, 6H), 1.52-1.43 (m, 2H), 1.39-1.31 (m, 4H), 1.27-1.17 (m, 6H).

### Synthesis of Tail 6-oxohexyl undec-10-enoate

Followed the synthesis method above. ¹H NMR (400 MHz, CDCl₃) δ 9.76 (t, J=1.7 Hz, 1H), 5.79 (ddt, J=16.9, 10.2, 6.7 Hz, 1H), 5.07-4.79 (m, 2H), 4.06 (t, J=6.6 Hz, 2H), 2.45 (td, J=7.3, 1.7 Hz, 2H), 2.28 (t, J=7.5 Hz, 2H), 2.08-1.95 (m, 2H), 1.71-1.55 (m, 6H), 1.45-1.37 (m, 2H), 1.36-1.12 (m, 9H).

### Synthesis of Tail 6-oxohexyl dodecanoate

Followed the synthesis method above. ¹H NMR (400 MHz, CDCl₃) δ 9.73 (t, J=1.8 Hz, 1H), 4.03 (t, J=6.6 Hz, 2H), 2.42 (td, J=7.3, 1.7 Hz, 2H), 2.25 (t, J=7.5 Hz, 2H), 1.62 (dd, J=15.3, 7.4 Hz, 6H), 1.37 (ddt, J=9.3, 6.7, 3.4 Hz, 2H), 1.22 (s, 16H), 0.83 (d, J=7.0 Hz, 3H).

### Synthesis of Tail 6-oxohexyl palmitate

Followed the synthesis method above. ¹H NMR (400 MHz, CDCl₃) δ 9.76 (t, J=1.6 Hz, 1H), 4.05 (t, J=6.6 Hz, 2H), 2.49-2.34 (m, 2H), 2.28 (t, J=7.5 Hz, 2H), 1.72-1.56 (m, 6H), 1.39 (tt, J=9.8, 6.3 Hz, 2H), 1.24 (s, 22H), 0.87 (t, J=6.7 Hz, 3H).

### Synthesis of Tail 6-oxohexyl stearate

Followed the synthesis method above. ¹H NMR (400 MHz, CDCl₃) δ 9.76 (t, J=1.7 Hz, 1H), 4.05 (t, J=6.6 Hz, 2H), 2.44 (td, J=7.3, 1.7 Hz, 2H), 2.28 (t, J=7.6 Hz, 2H), 1.72-1.53 (m, 6H), 1.48-1.06 (m, 32H), 0.91-0.78 (m, 3H).

### Synthesis of Tail 6-oxohexyl oleate

Followed the synthesis method above. ¹H NMR (400 MHz, CDCl₃) δ 9.75 (t, J=1.7 Hz, 1H), 5.37-5.27 (m, 2H), 4.04 (t, J=6.6 Hz, 2H), 2.43 (td, J=7.3, 1.7 Hz, 2H), 2.27 (t, J=7.6 Hz, 2H), 1.99 (q, J=6.7 Hz, 3H), 1.71-1.54 (m, 6H), 1.43-1.34 (m, 2H), 1.32-1.19 (m, 20H), 0.87-0.81 (m, 3H).

### General Synthesis Route of Tail C

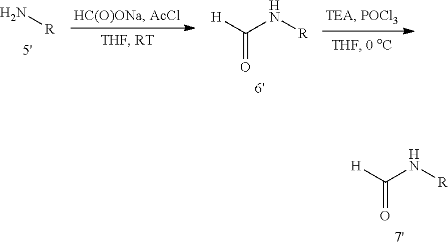

### Amines (5′) Used:

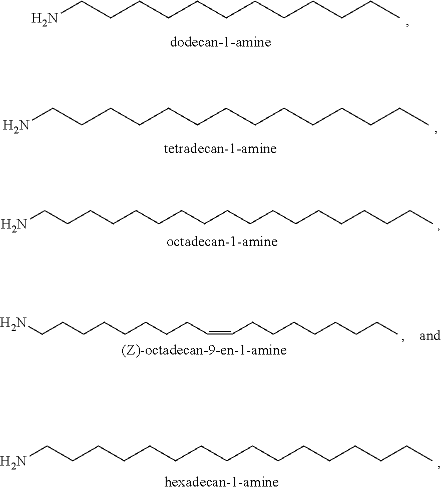 [SMILES: CCCCCCCCCCCCN ; CCCCCCCCCCCCCCN ; CCCCCCCCCCCCCCCCCCN ; CCCCCCCC/C=C\CCCCCCCCN ; CCCCCCCCCCCCCCCCN]

Tetrahydrofuran (82.75 ml) and solid formate (2.2511 g) were added into a 250 mL round bottom flask and stirred to form a suspension. A DCM (34.755 mL) solution containing acetyl chloride (1.0 M) was taken in a constant pressure titration funnel and added dropwise to the suspension prepared above. The mixture was stirred at room temperature for 12 h after the dropwise addition to obtain reaction solution 1. 5′ was dissolved in THE and added dropwise to reaction solution 1. The mixture was stirred at room temperature for 4 h after the dropwise addition to obtain reaction solution 2. Reaction solution 2 was diluted with 125 ml of deionized water, and the aqueous solution was extracted twice with 240 ml of ethyl acetate, each with an amount of 120 ml. The resulting extract was dried with anhydrous magnesium sulphate for 0.5 h and spin dried to obtain the crude 6′.

The crude 6′ product and THE (50 mL) solution were added to a round-bottomed flask (100 mL) and placed in an ice bath at 0° C. The mixture was stirred for 10 min and TEA was added to the solution. The mixture was stirred in the ice bath for 15 min to obtain the reaction solution 1. THE (12.9 mL) and phosphorus oxychloride (3.519 g) were taken to prepare solution 1. Under ice bath conditions, solution 1 was added slowly dropwise to reaction solution 1 with a constant pressure-dropping funnel. The reaction was stirred for 1.5 h. 65 mL of a saturated sodium bicarbonate solution was added to dilute and wash the mixture, and the washed solution was extracted twice with 130 mL of ether, 65 mL each time. The extracts were combined, and anhydrous magnesium sulphate was added to the organic phase, stirred and dried for 0.5 h. The solution was removed by rotary evaporation. The crude product (7′) was purified by column using a Hexane/EA system.

### Synthesis of Tail 1-isocyanododecane

Followed the synthesis method above. ¹H NMR (400 MHz, CDCl₃) δ 3.49-3.22 (m, 2H), 1.72-1.57 (m, 2H), 1.50-1.34 (m, 2H), 1.26 (d, J=10.0 Hz, 16H), 0.89-0.84 (m, 3H).

### Synthesis of Tail 1-isocyanotetradecane

Followed the synthesis method above. ¹H NMR (400 MHz, CDCl₃) δ 3.47-3.24 (m, 2H), 1.66 (ddt, J=10.8, 8.4, 4.2 Hz, 2H), 1.46-1.37 (m, 2H), 1.27 (d, J=11.2 Hz, 20H), 0.94-0.74 (m, 3H).

### Synthesis of Tail 1-isocyanohexadecane

Followed the synthesis method above. ¹H NMR (400 MHz, CDCl₃) δ 3.45-3.26 (m, 2H), 1.66 (ddtt, J=11.4, 6.7, 4.6, 2.3 Hz, 2H), 1.46-1.38 (m, 2H), 1.26 (d, J=11.1 Hz, 24H), 0.87 (t, J=6.8 Hz, 3H).

### Synthesis of Tail 1-isocyanooctadecane

Followed the synthesis method above. ¹H NMR (400 MHz, CDCl₃) δ 3.48-3.23 (m, 2H), 1.73-1.61 (m, 2H), 1.46-1.37 (m, 2H), 1.25 (s, 28H), 0.91-0.84 (m, 3H).

### Synthesis of Tail (Z)-1-isocyanooctadec-9-ene

Followed the synthesis method above. ¹H NMR (400 MHz, CDCl₃) δ 5.41-5.21 (m, 2H), 3.45-3.29 (m, 2H), 1.99 (dq, J=14.2, 6.0 Hz, 3H), 1.66 (ddd, J=8.4, 5.6, 3.4 Hz, 2H), 1.48-1.35 (m, 2H), 1.28 (dt, J=17.3, 6.2 Hz, 20H), 0.92-0.84 (m, 3H).

### Tail Synthesis for ML-Predicted Structure

### Synthesis of olealdehyde

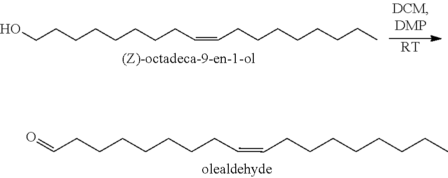

Olealdehyde was made using the same procedure as the synthesis method above. ¹H NMR (400 MHz, CDCl₃) δ 9.75 (t, J=1.9 Hz, 1H), 5.34 (qd, J=3.8, 1.7 Hz, 2H), 2.41 (td, J=7.4, 1.9 Hz, 2H), 2.00 (q, J=6.1 Hz, 4H), 1.62 (p, J=7.3 Hz, 2H), 1.34-1.22 (m, 20H), 0.91-0.83 (m, 3H).

### Synthesis of palmitaldehyde

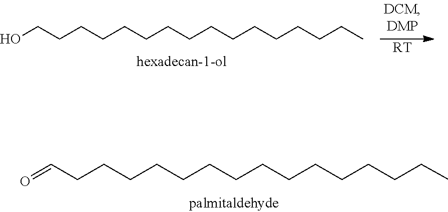

Palmitaldehyde was made using the same procedure as the synthesis method above. ¹H NMR (400 MHz, CDCl₃) δ 9.76 (s, 1H), 2.41 (td, J=7.4, 1.9 Hz, 2H), 1.62 (dd, J=9.3, 5.2 Hz, 2H), 1.25 (s, 23H), 0.90-0.84 (m, 3H).

### Synthesis of 4-oxobutyl 4-methylnonanoate

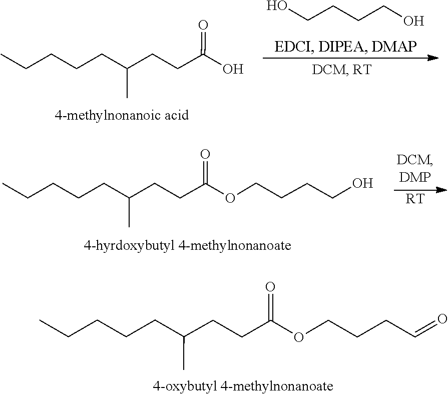

4-hydroxybutyl 4-methylnonanoate was made using the same procedure as the synthesis method above. ¹H NMR (400 MHz, CDCl₃) δ 4.09-4.04 (m, 2H), 3.64 (t, J=6.3 Hz, 2H), 2.27 (td, J=9.3, 6.1 Hz, 2H), 1.70-1.57 (m, 4H), 1.45-1.15 (m, 10H), 0.90-0.77 (m, 6H). 4-oxobutyl 4-methylnonanoate was made using the same procedure as the synthesis method above. ¹H NMR (400 MHz, CDCl₃) δ 9.78 (s, 1H), 4.08 (t, J=6.3 Hz, 2H), 2.53 (td, J=7.2, 1.3 Hz, 2H), 2.28 (td, J=9.4, 6.1 Hz, 2H), 2.01-1.90 (m, 2H), 1.66-1.57 (m, 1H), 1.44-1.19 (m, 10H), 0.88-0.83 (m, 6H).

### Synthesis of 4-oxobutyl (E)-dec-2-enoate

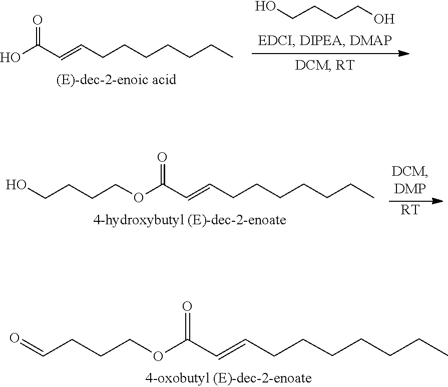

4-hydroxybutyl (E)-dec-2-enoate was made using the same procedure as the synthesis method above. ¹H NMR (400 MHz, CDCl₃) δ 6.89 (dt, J=15.6, 6.9 Hz, 1H), 5.73 (dt, J=15.7, 1.6 Hz, 1H), 4.08 (t, J=6.5 Hz, 2H), 3.58 (t, J=6.4 Hz, 2H), 2.12 (qd, J=7.1, 1.6 Hz, 2H), 1.71-1.54 (m, 5H), 1.41-1.34 (m, 2H), 1.24-1.19 (m, 8H), 0.80 (d, J=7.1 Hz, 3H).

4-oxobutyl (E)-dec-2-enoate was made using the same procedure as the synthesis method above. ¹H NMR (400 MHz, CDCl₃) δ 9.78 (t, J=1.3 Hz, 1H), 6.95 (dt, J=15.6, 6.9 Hz, 1H), 5.78 (d, J=15.7 Hz, 1H), 4.15 (t, J=6.3 Hz, 2H), 2.55 (td, J=7.2, 1.3 Hz, 2H), 2.18 (qd, J=7.1, 1.6 Hz, 2H), 1.99 (ddd, J=13.6, 7.3, 6.3 Hz, 2H), 1.46-1.38 (m, 2H), 1.29-1.23 (m, 8H), 0.88-0.84 (m, 3H).

### Synthesis of 4-oxobutyl octanoate

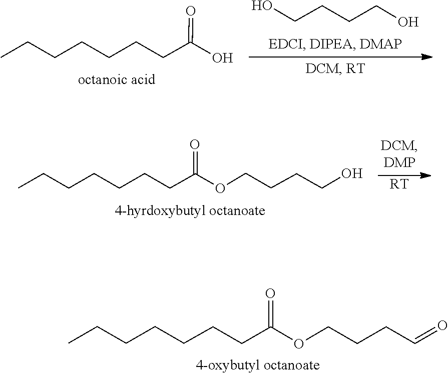

4-hydroxybutyl octanoate was made using the same procedure as the synthesis method above. ¹H NMR (400 MHz, CDCl₃) δ 4.06 (q, J=7.1 Hz, 2H), 3.67 (p, J=6.7 Hz, 1H), 3.08 (q, J=7.4 Hz, 1H), 2.23-2.19 (m, 3H), 1.31 (d, J=6.8 Hz, 7H), 1.22-1.20 (m, 9H), 0.82 (d, J=4.0 Hz, 6H). 4-oxobutyl octanoate was made using the same procedure as the synthesis method above. ¹H NMR (400 MHz, CDCl₃) δ 9.77 (t, J=1.3 Hz, 1H), 4.08 (t, J=6.3 Hz, 2H), 2.53 (td, J=7.2, 1.3 Hz, 2H), 2.30-2.22 (m, 2H), 1.96 (ddd, J=13.5, 7.2, 6.3 Hz, 2H), 1.67-1.53 (m, 2H), 1.31-1.21 (m, 9H), 0.88-0.83 (m, 3H).

### Synthesis of 4-oxobutyl oleate

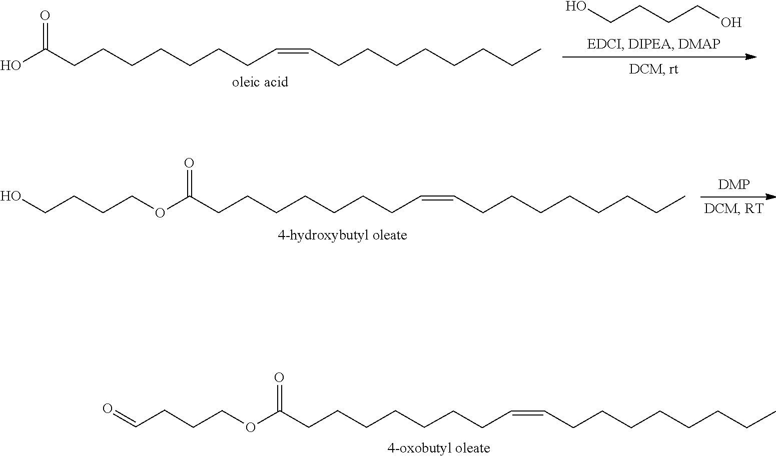 [SMILES: CCCCCCCC/C=C\CCCCCCCC(=O)O ; OCCCCO ; CCCCCCCC/C=C\CCCCCCCC(=O)OCCCCO ; CCCCCCCC/C=C\CCCCCCCC(=O)OCCCC=O]

4-hydroxybutyl oleate was made using the same procedure as the synthesis method above. ¹H NMR (400 MHz, CDCl₃) δ 5.42-5.25 (m, 2H), 4.12-4.09 (m, 2H), 3.68 (t, J=6.3 Hz, 2H), 2.29 (t, J=7.6 Hz, 2H), 2.00 (q, J=6.5 Hz, 4H), 1.76-1.60 (m, 6H), 1.31-1.24 (m, 20H), 0.87 (t, J=6.7 Hz, 3H). 4-oxobutyl oleate was made using the same procedure as the synthesis method above. ¹H NMR (400 MHz, CDCl₃) δ 9.70 (t, J=1.3 Hz, 1H), 5.45-5.15 (m, 2H), 4.14 (m, 2H), 2.52-2.18 (m, 4H), 2.12-1.78 (m, 6H), 1.70-1.50 (m, 2H), 1.41-1.11 (m, 20H), 1.02-0.78 (m, 3H).

### Synthesis of 1-isocyanoundecane

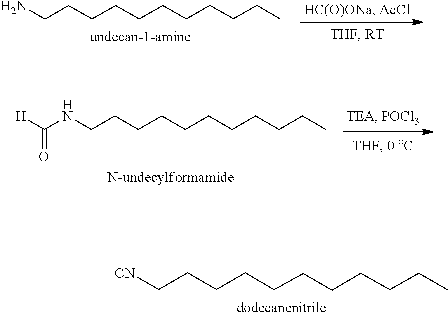

1-isocyanoundecane was made using the same procedure as the synthesis method above. ¹H NMR (400 MHz, CDCl₃) δ 3.42-3.27 (m, 2H), 1.63-1.52 (m, 2H), 1.31-1.24 (m, 16H), 0.88-0.86 (m, 3H).

### Synthesis of 1-isocyanoheptadecane

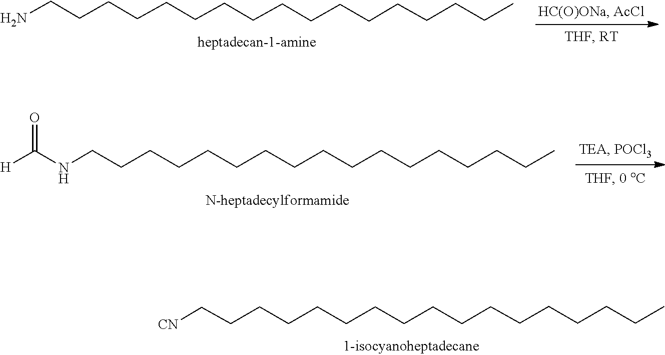

1-isocyanoheptadecane was made using the same procedure as the synthesis method above. ¹H NMR (400 MHz, CDCl₃) δ 3.44-3.29 (m, 2H), 1.74-1.37 (m, 4H), 1.25 (d, J=1.5 Hz, 15H), 0.87 (td, J=6.9, 1.8 Hz, 3H).

### Top-Performing Lipid Synthesis

High-Throughput Synthesis and Screening of a Combinatorial Ionizable Lipid Library for mRNA Delivery

The ionizable lipid structures were deconstructed into three distinct components: an amine head group, lipid tail A, and lipid tail B, aligning with the three reactants in a standard 3CR-Ugi reaction (amine, isocyanide, aldehyde), respectively. An ionizable lipid can thus be readily synthesized by simultaneously coupling the amine headgroup (A) to the aldehyde tail (B) and the isocyanide tail (C) as shown in the following Scheme:

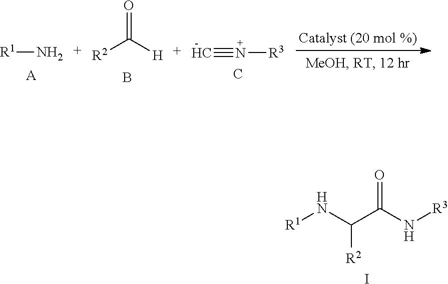

This design differentiates itself from conventional 3CR methodologies, which typically categorize ionizable lipids into three segments: amine head group, linker, and lipid tail. This new 3CR synthetic approach places a stronger emphasis on the significance of lipid tails by introducing an additional dimension to their design. This enables the exploration of a unique chemical space for ionizable lipids with diverse tails, accommodating both symmetrical and asymmetrical structures.

To address the limitation of low yield encountered in the previous method of lipid generation using the 3-CR-Ugi reaction, an investigation into various catalysts, including p-toluenesulfonic acid, phosphoric acid, boric acid, and phenylphosphinic acid for boosting the reaction efficiency. Remarkably, phenylphosphinic acid (20% mol) emerged as the most effective catalyst, increasing the reaction yield from 10% to 70% (See Table below). To validate this finding, multiple lipids were synthesized and purified, consistently showing high yields ranging from 75% to 90%. This improvement allow the establishment of a reliable platform for high throughput synthesis and screening of ionizable lipid libraries.

TABLE
Optimization of the reaction conditions of the test product

| Entry | Lewis acid | Condition | Solvent | Yield (%)^{a} |
|---|---|---|---|---|
| 1 | N/A | rt/48 | MeOH | 10 |
| 2 | TsOH (20%) | rt/24 | MeOH | 42 |
| 3 | H₃PO₄ (20%) | rt/24 | MeOH | 50 |
| 4 | H₃BO₄ (20%) | rt/24 | MeOH | 38 |
| 5 | 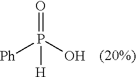 | rt/24 | MeOH | 75 |

a: isolated yield

### Synthesis Method of Lipid 1-2 (H278) (Z)-5-(octadec-9-en-1-ylamino)-5-oxo-4-(piperidin-1-ylamino)pentyl octanoate (I-2)

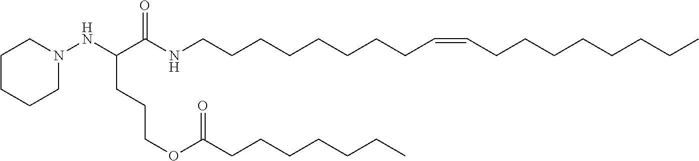 [SMILES: CCCCCCCC/C=C\CCCCCCCCNC(=O)C(CCCOC(=O)CCCCCCC)NN1CCCCC1]

The synthesis of 1-2 or H278 through 3CR-Ugi reaction. A mixture of piperidin-1-amine (25 mg, 249.59 μmol), 4-oxobutyl octanoate (53.49 mg, 249.59 μmol) and (Z)-1-isocyanooctadec-9-ene (69.26 mg, 249.59 μmol) in anhydrous solvent DCM (240 μL) and MeOH (160 μL) with the catalyst phosphoric acid (7.09 mg, 49.92 μmol) and was stirred in capped glass vials at room temperature overnight. The mixture was purified by flash column chromatography (0.1% ammonia hydroxide with 0-10% MeOH in DCM) to obtain compound 1-2 (H278) as colorless oil. (35.0 mg, 59.13 μmol, 23.69%). ¹H NMR (400 MHz, CDCl₃) δ 7.03 (t, J=5.8 Hz, 1H), 5.46-5.31 (m, 2H), 4.05 (t, J=6.1 Hz, 2H), 3.40-3.26 (m, 2H), 3.23-3.11 (m, 1H), 2.69 (s, 2H), 2.41 (s, 2H), 2.28 (t, J=7.6 Hz, 2H), 1.99 (dd, J=14.1, 7.7 Hz, 4H), 1.74-1.46 (m, 14H), 1.33-1.21 (m, 31H), 0.91-0.83 (m, 6H). Chemical Formula: C₃₆H₆₉N₃O₃ and MS calculated: m/z 591.53, observed (ESI ms) m/z 592.53.

### Synthesis Method of ((Z)-7-((1H-pyrazol-4-yl)amino)-8-(octadec-9-en-1-ylamino)-8-oxooctyl nonanoate) (I-7)

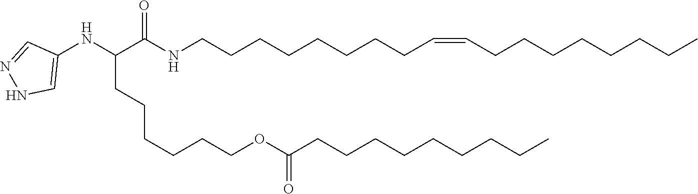 [SMILES: CCCCCCCC/C=C\CCCCCCCCNC(=O)C(CCCCCCOC(=O)CCCCCCCCC)Nc1cn[nH]c1]

The synthesis of the top-performing lipid in 1,200 screening through 3CR-Ugi reaction. A mixture of piperidin-1-amine (50 mg, 601.73 μmol), 6-oxobutyl octanoate (160.96 mg, 601.73 μmol) and (Z)-1-isocyanooctadec-9-ene (154.27 mg, 601.73 μmol) in anhydrous solvent DCM (240 μL) and MeOH (160 μL) and was stirred in capped glass vials at room temperature overnight. The mixture was purified by flash column chromatography (0.1% ammonia hydroxide with 0-10% MeOH in DCM) to obtain finial compound as colorless oil. (285.0 mg, 451.66 μmol, 75.06%). ¹H NMR (400 MHz, CDCl₃) δ 7.32-7.04 (m, 2H), 6.92 (t, J=4.9 Hz, 1H), 5.33 (p, J=3.6 Hz, 2H), 4.10 (tt, J=5.6, 4.1 Hz, 3H), 3.25 (td, J=5.9, 4.8 Hz, 2H), 2.39-2.24 (m, 3H), 2.03-1.90 (m, 4H), 1.68-1.53 (m, 3H), 1.48-1.20 (m, 41H), 0.94-0.81 (m, 6H).

### Synthesis Method of Lipid R080 2-((3-(diethylamino)propyl) amino)-N-undecylheptadecanamide (I-3)

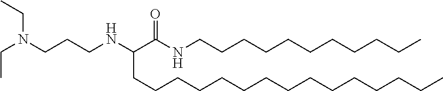 [SMILES: CCCCCCCCCCCCCCCC(NCCCN(CC)CC)C(=O)NCCCCCCCCCCC]

The synthesis of R080 through 3CR-Ugi reaction. A mixture of N, N-diethylpropane-1,3-diamine (25 mg, 191.96 μmol), palmitaldehyde (46.15 mg, 191.96 μmol) and 1-isocyanoundecane (34.81 mg, 191.96 μmol) in anhydrous solvent DCM (240 μL) and MeOH (160 μL) with the catalyst phosphoric acid (5.46 mg, 39.39 μmol) and was stirred in capped glass vials at room temperature overnight. The mixture was purified by flash column chromatography (0.1% ammonia hydroxide with 0-10% MeOH in DCM) to obtain compound R080 as colorless oil. (39.8 mg, 72.1 μmol, 37.56%). ¹H NMR (400 MHz, CDCl₃) δ 3.27-3.18 (m, 2H), 2.99 (dd, J=7.7, 4.7 Hz, 1H), 2.67-2.40 (m, 8H), 1.73-1.43 (m, 6H), 1.33-1.19 (m, 44H), 1.03 (td, J=7.1, 3.1 Hz, 7H), 0.91-0.84 (m, 6H). Chemical Formula: C₃₅H₇₃N₃O and MS calculated: m/z 551.58, observed (ESI ms) m/z 552.73.

### Synthesis Method of (Z)-2-((3-(dimethylamino)propyl)amino)-N-(octadec-9-en-1-yl)undecanamide (I-6)

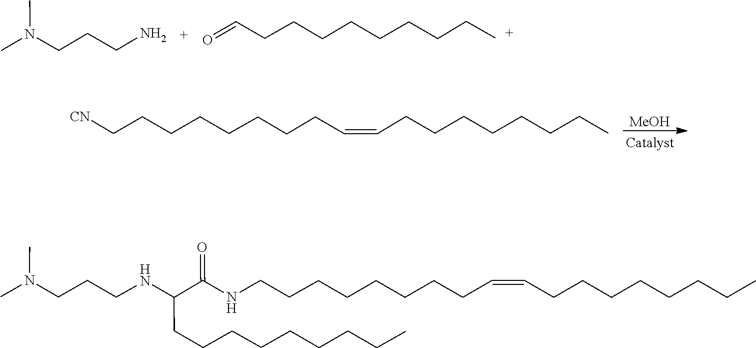

Follow the synthesis method above. The residue was purified by silica gel chromatography (0.1% ammonia hydroxide with 0-10% MeOH in DCM) to give the title lipid (Yield: 75%, determined by combi flash of BUCHI purification system) as a colorless oil. ¹H NMR (400 MHz, CDCl₃) δ 5.37-5.27 (m, 2H), 2.70 (t, J=7.2 Hz, 3H), 2.21 (d, J=24.8 Hz, 9H), 2.04-1.92 (m, 5H), 1.46 (d, J=7.3 Hz, 3H), 1.39-1.15 (m, 39H), 0.88 (t, J=6.7 Hz, 6H).

### Synthesis Method of ((Z)-2-((3-(dimethylamino)propyl)amino)-N-(octadec-9-en-1-yl)heptadecanamide (I-5)

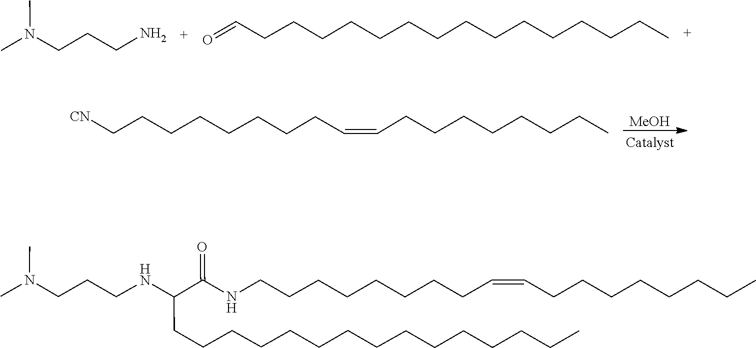

Follow the synthesis method above. The residue was purified by silica gel chromatography (0.1% ammonia hydroxide with 0-10% MeOH in DCM) to give the title lipid (Yield: 90%, determined by combi flash of BUCHI purification system) as a colorless oil. ¹H NMR (400 MHz, CDCl₃) δ 5.36-5.32 (m, 2H), 3.86 (s, 3H), 3.25 (dq, J=34.4, 6.7 Hz, 4H), 2.07-1.91 (m, 6H), 1.67-1.46 (m, 4H), 1.44-0.94 (m, 53H), 0.87 (t, J=6.7 Hz, 6H).

### Synthesis Method of (Z)—N-(octadec-9-en-1-yl)-2-((2-(pyrrolidin-1-yl)ethyl)amino)heptadecanamide (I-4)

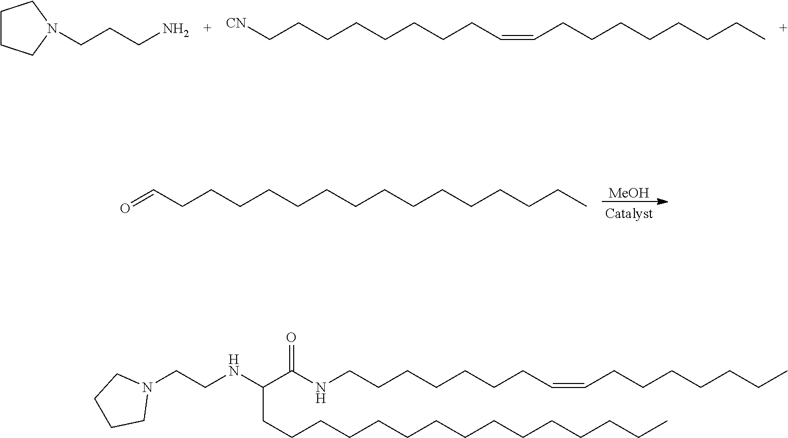

Follow the synthesis method above. The residue was purified by silica gel chromatography (0.1% ammonia hydroxide with 0-10% MeOH in DCM) to give the title lipid (Yield: 85%, determined by combi flash of BUCHI purification system) as a colorless oil ¹H NMR (400 MHz, CDCl₃) δ 5.35-5.33 (m, 2H), 2.67 (t, J=7.0 Hz, 4H), 2.00 (q, J=6.5 Hz, 7H), 1.59-1.10 (m, 57H), 0.87 (t, J=6.7 Hz, 6H).

### Combinatorial Lipid Library

To efficiently create diverse libraries of ionizable lipids, a high-throughput synthesis (HTS) platform, leveraging the one-pot Ugi 3-CR method (FIG. 6). Automated robotic liquid handling facilitated the synthesis of 1,200 ionizable lipids in just a day. The experimental lipid library comprised 20 unique head groups, 12 alkyl chains with biodegradable ester linkages, and 5 alkyl chains with isocyanide functional groups as follows:

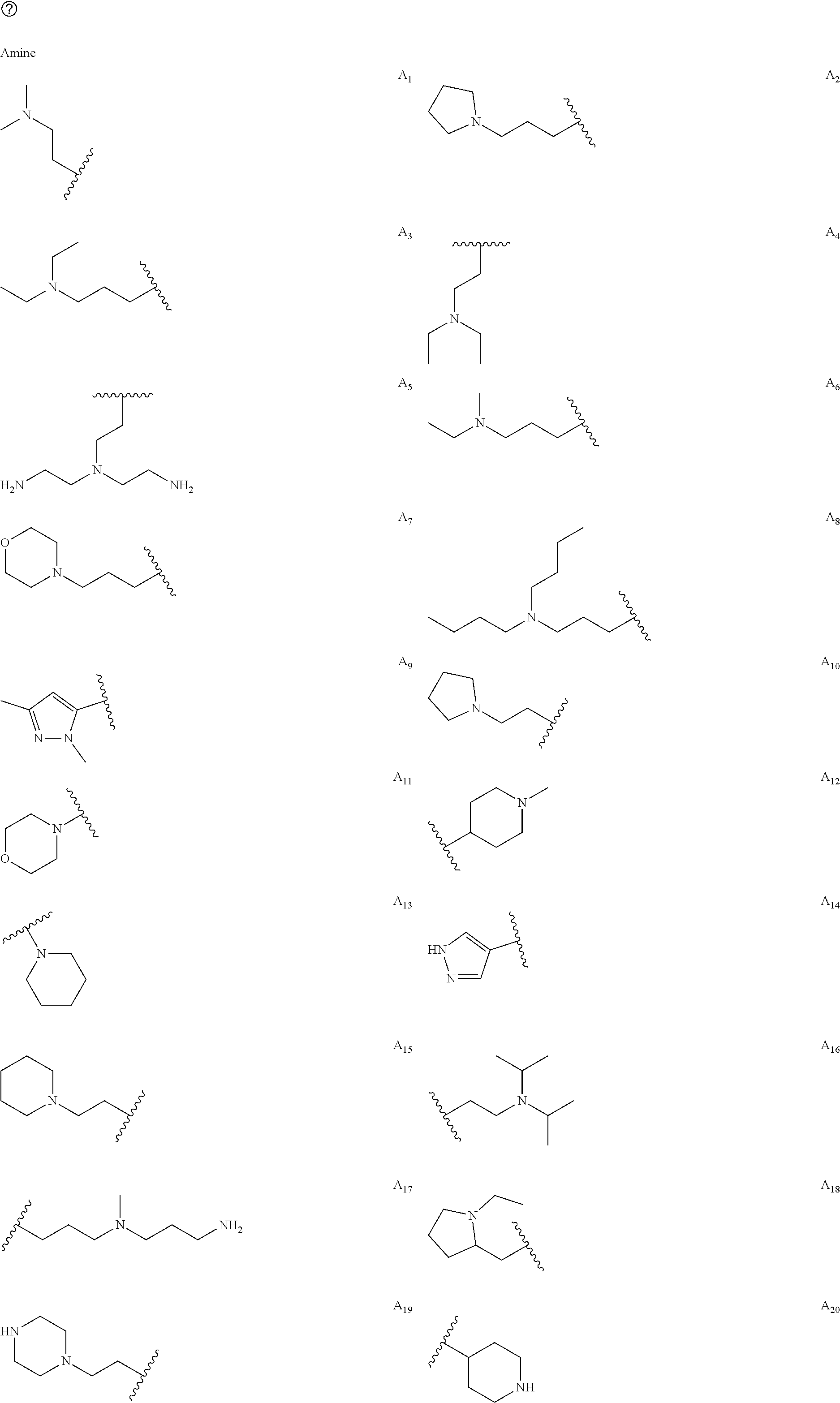

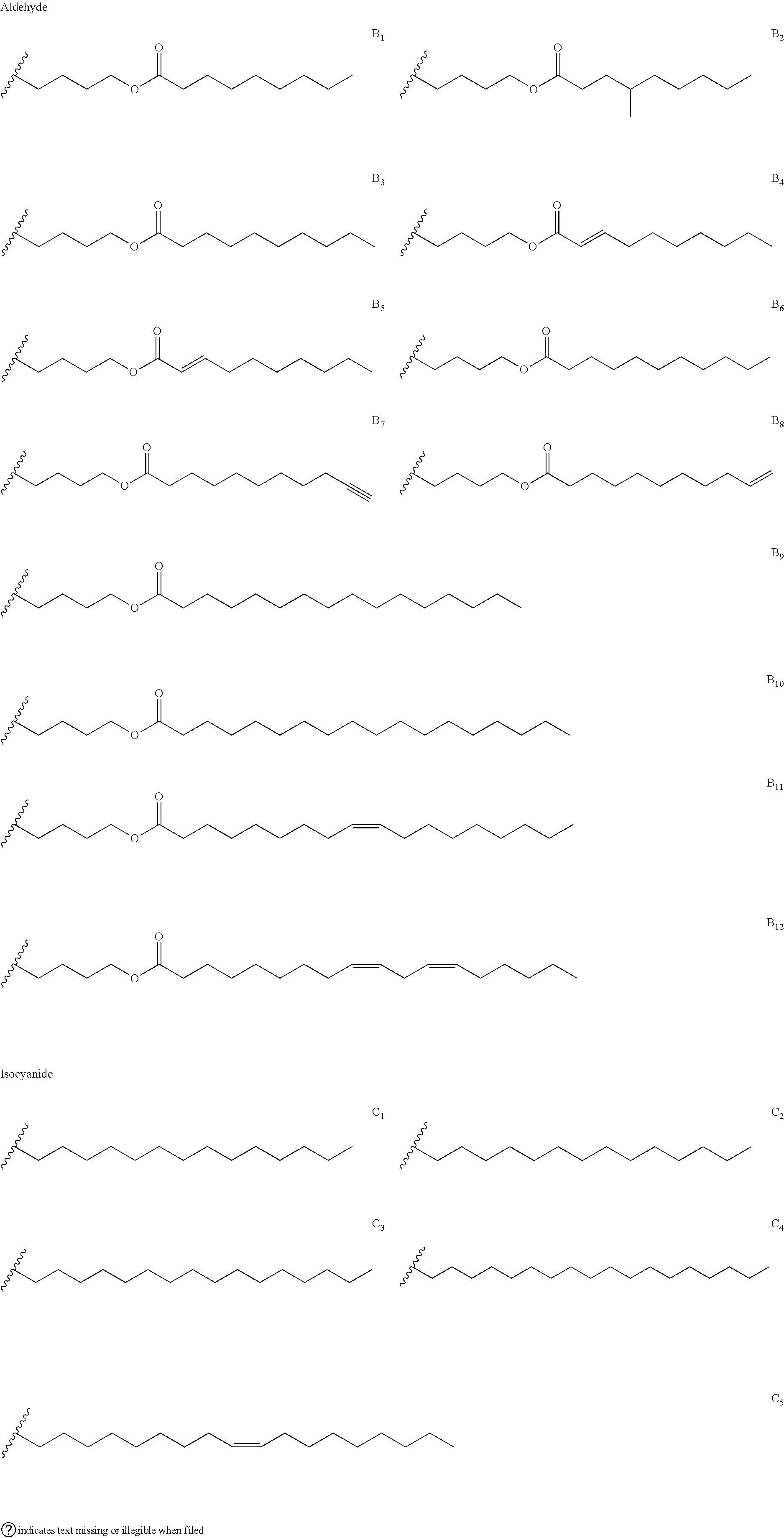

This automation also enabled rapid formulation of these new ionizable lipids into lipid nanoparticles (LNPs) using a standardized four-component formulation ratio, reducing operator variability (FIG. 7). The LNPs were loaded with firefly luciferase mRNA (mFluc) to quickly assess mTP in target cells, which herein was measured as the base 2 logarithm of the luminescence intensity ratio between transfected and untreated cells 24 hours post-treatment, a timeframe chosen to capture peak bioluminescent signal based on mFluc expression kinetics in cells. The entire processes of lipid synthesis, LNP formulation, and luciferase assay were streamlined and automated using the liquid handling robot. To validate the suitability of the automated liquid processor for LNP formulation, it was compared against the conventional manual pipette method. Analysis of the results found no significant differences between the automated liquid processor and manual pipette in terms of LNP properties.

The following additional lipids were prepared in a like manner:

| | Chemical | | Calcd. | Ob- |
|---|---|---|---|---|
| Cpd | Formula | Structure | (m/z) | served |
| I-1 or iso- A11B5C1 | C₄₁H₇₉N₃O₃ | 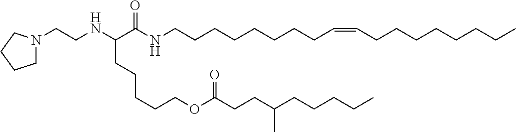 [SMILES: CCCCCCCC/C=C\CCCCCCCCNC(=O)C(CCCCCOC(=O)CCC(C)CCCCC)NCCN1CCCC1] | 662.10 | |
| H2 (I-8) | C₃₅H₆₉N₃O₃ | 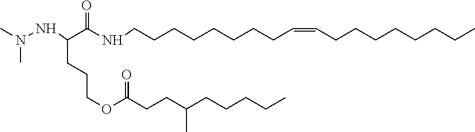 [SMILES: CCCCCCCC/C=C\CCCCCCCCNC(=O)C(CCCOC(=O)CCC(C)CCCCC)NN(C)C] | 580.53 | 580.56 |
| H3 (I-9) | C₃₉H₇₇N₃O | 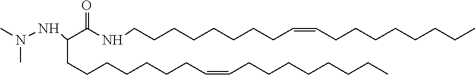 [SMILES: CCCCCCCC/C=C\CCCCCCCCNC(=O)C(CCCCCCC/C=C\CCCCCCCC)NN(C)C] | 604.61 | 604.82 |
| H4 (I-10) | C₄₄H₈₇N₃O₃ | 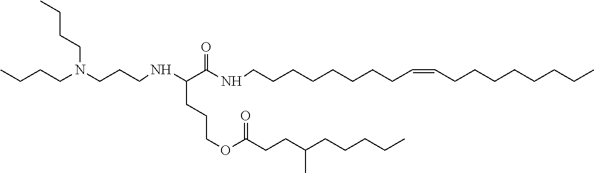 [SMILES: CCCCCCCC/C=C\CCCCCCCCNC(=O)C(CCCOC(=O)CCC(C)CCCCC)NCCCN(CCCC)CCCC] | 706.67 | 706.04 |
| H5 (I-11) | C₄₁H₈₃N₃O | 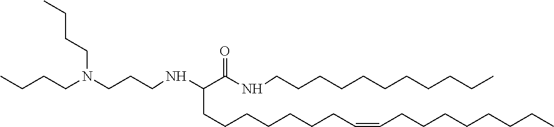 [SMILES: CCCCCCCC/C=C\CCCCCCCC(NCCCN(CCCC)CCCC)C(=O)NCCCCCCCCCCC] | 634.65 | 634.64 |
| H6 (I-12) | C₄₄H₈₅N₃O₃ | 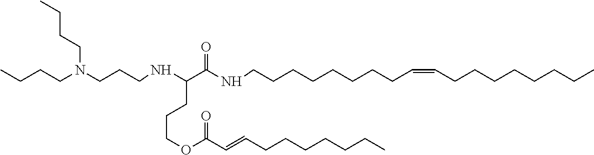 [SMILES: CCCCCCC/C=C/C(=O)OCCCC(NCCCN(CCCC)CCCC)C(=O)NCCCCCCCC/C=C\CCCCCCCC] | 704.66 | 704.55 |
| H7 (I-13) | C₃₈H₇₃N₃O₃ | 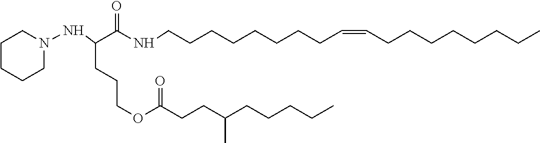 [SMILES: CCCCCCCC/C=C\CCCCCCCCNC(=O)C(CCCOC(=O)CCC(C)CCCCC)NN1CCCCC1] | 620.57 | 620.03 |
| H8 (I-14) | C₃₈H₇₁N₃O₃ | 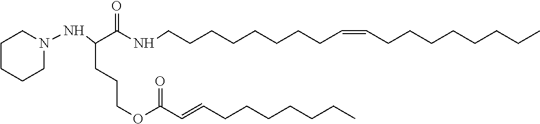 [SMILES: CCCCCCC/C=C/C(=O)OCCCC(NN1CCCCC1)C(=O)NCCCCCCCC/C=C\CCCCCCCC] | 641.55 | 641.55 |
| H9 (I-15) | C₃₆H₆₉N₃O₃ | 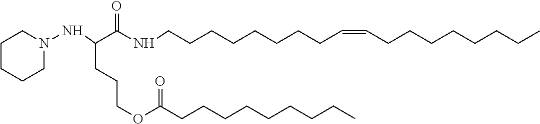 [SMILES: CCCCCCCC/C=C\CCCCCCCCNC(=O)C(CCCOC(=O)CCCCCCCCC)NN1CCCCC1] | 592.53 | 592.53 |
| H10 (I-16) | C₄₁H₈₁N₃O₃ | 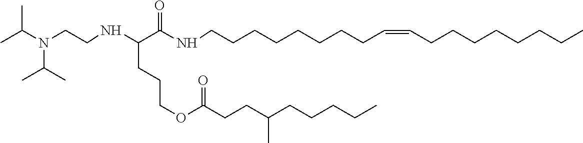 [SMILES: CCCCCCCC/C=C\CCCCCCCCNC(=O)C(CCCOC(=O)CCC(C)CCCCC)NCCN(C(C)C)C(C)C] | 664.63 | 664.94 |
| H11 (I-17) | C₄₁H₈₀N₃O₃ | 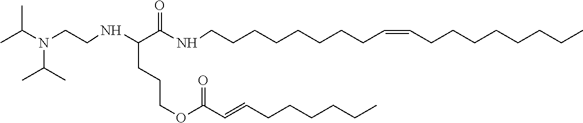 [SMILES: CCCCCC/C=C/C(=O)OCCCC(NCCN(C(C)C)C(C)C)C(=O)NCCCCCCCC/C=C\CCCCCCCC] | 663.61 | 663.29 |
| H12 (I-18) | C₃₇H₇₅N₃O₃ | 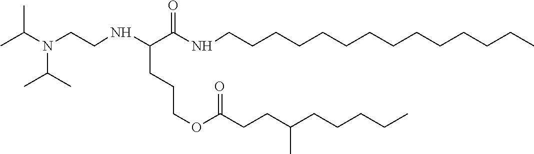 [SMILES: CCCCCCCCCCCCCCNC(=O)C(CCCOC(=O)CCC(C)CCCCC)NCCN(C(C)C)C(C)C] | 610.58 | 610.61 |
| H13 (I-19) | C₄₀H₇₉N₃O₃ | 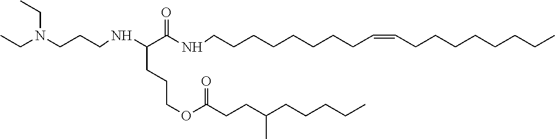 [SMILES: CCCCCCCC/C=C\CCCCCCCCNC(=O)C(CCCOC(=O)CCC(C)CCCCC)NCCCN(CC)CC] | 650.61 | 650.52 |
| H14 (I-20) | C₄₀H₇₇N₃O₃ | 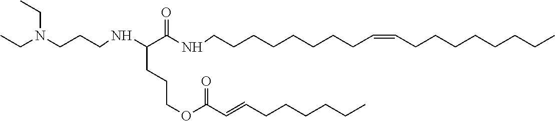 [SMILES: CCCCCC/C=C/C(=O)OCCCC(NCCCN(CC)CC)C(=O)NCCCCCCCC/C=C\CCCCCCCC] | 648.60 | 648.85 |
| H15 (I-21) | C₄₈H₉₃N₃O₃ | 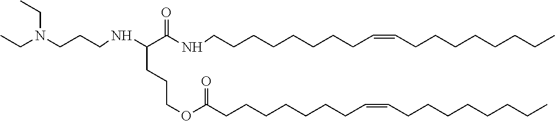 [SMILES: CCCCCCCC/C=C\CCCCCCCCNC(=O)C(CCCOC(=O)CCCCCCC/C=C\CCCCCCCC)NCCCN(CC)CC] | 760.72 | 760.52 |
| H31 (I-22) | C₄₀H₇₇N₃O₃ | 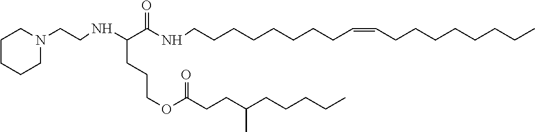 [SMILES: CCCCCCCC/C=C\CCCCCCCCNC(=O)C(CCCOC(=O)CCC(C)CCCCC)NCCN1CCCCC1] | 647.60 | 647.60 |
| H32 (I-23) | C₄₀H₇₅N₃O₃ | 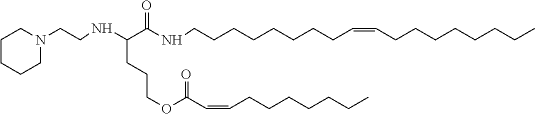 [SMILES: CCCCCCC/C=C\C(=O)OCCCC(NCCN1CCCCC1)C(=O)NCCCCCCCC/C=C\CCCCCCCC] | 645.58 | 645.82 |
| H33 (I-24) | C₄₀H₇₉N₃O₃ | 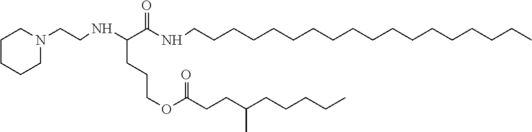 [SMILES: CCCCCCCCCCCCCCCCCCNC(=O)C(CCCOC(=O)CCC(C)CCCCC)NCCN1CCCCC1] | 649.61 | 649.14 |
| H34 (I-25) | C₄₆H₈₉N₃O₃ | 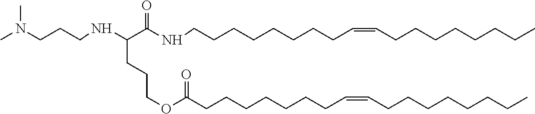 [SMILES: CCCCCCCC/C=C\CCCCCCCCNC(=O)C(CCCOC(=O)CCCCCCC/C=C\CCCCCCCC)NCCCN(C)C] | 731.69 | 731.85 |
| H35 (I-26) | C₃₈H₇₅N₃O₃ | 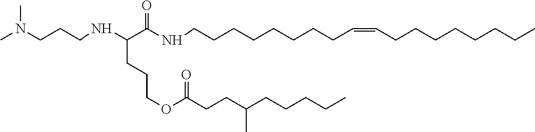 [SMILES: CCCCCCCC/C=C\CCCCCCCCNC(=O)C(CCCOC(=O)CCC(C)CCCCC)NCCCN(C)C] | 621.58 | 621.34 |
| H36 (I-27) | C₃₈H₇₃N₃O₃ | 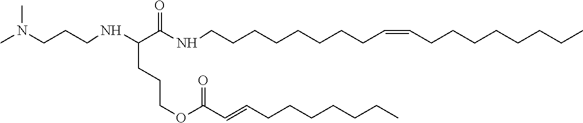 [SMILES: CCCCCCC/C=C/C(=O)OCCCC(NCCCN(C)C)C(=O)NCCCCCCCC/C=C\CCCCCCCC] | 619.57 | 619.79 |
| H37 (I-28) | C₃₇H₇₃N₃O₃ | 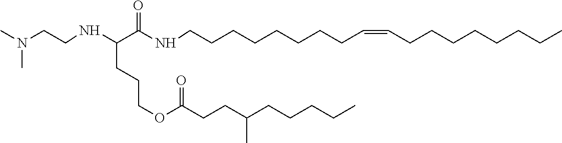 [SMILES: CCCCCCCC/C=C\CCCCCCCCNC(=O)C(CCCOC(=O)CCC(C)CCCCC)NCCN(C)C] | 607.57 | 607.66 |
| H38 (I-29) | C₃₃H₆₇N₃O₃ | 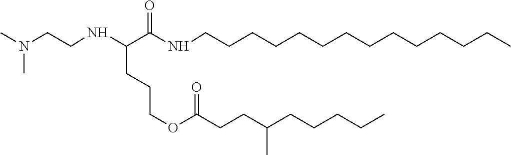 [SMILES: CCCCCCCCCCCCCCNC(=O)C(CCCOC(=O)CCC(C)CCCCC)NCCN(C)C] | 553.52 | 553.92 |
| H39 (I-30) | C₃₇H₇₁N₃O₃ | 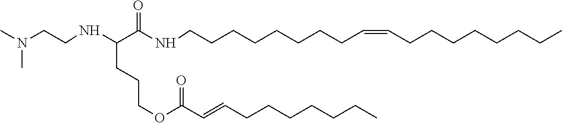 [SMILES: CCCCCCC/C=C/C(=O)OCCCC(NCCN(C)C)C(=O)NCCCCCCCC/C=C\CCCCCCCC] | 605.55 | 605.69 |
| H40 (I-31) | C₄₁H₇₉N₃O₃ | 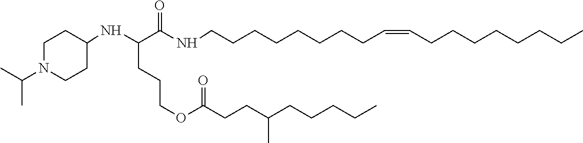 [SMILES: CCCCCCCC/C=C\CCCCCCCCNC(=O)C(CCCOC(=O)CCC(C)CCCCC)NC1CCN(C(C)C)CC1] | 661.61 | 661.61 |
| H41 (I-32) | C₄₁H₇₇N₃O₃ | 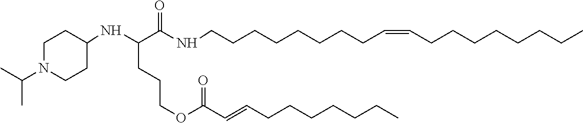 [SMILES: CCCCCCC/C=C/C(=O)OCCCC(NC1CCN(C(C)C)CC1)C(=O)NCCCCCCCC/C=C\CCCCCCCC] | 659.60 | 659.22 |
| H42 (I-33) | C₄₁H₈₁N₃O₃ | 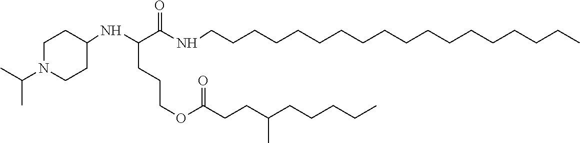 [SMILES: CCCCCCCCCCCCCCCCCCNC(=O)C(CCCOC(=O)CCC(C)CCCCC)NC1CCN(C(C)C)CC1] | 663.63 | 663.36 |
| H43 (I-34) | C₄₀H₇₇N₃O₄ | 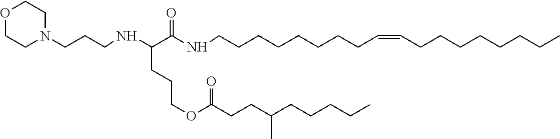 [SMILES: CCCCCCCC/C=C\CCCCCCCCNC(=O)C(CCCOC(=O)CCC(C)CCCCC)NCCCN1CCOCC1] | 663.59 | 663.08 |
| H44 (I-35) | C₄₀H₇₅N₃O₄ | 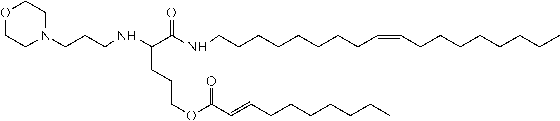 [SMILES: CCCCCCC/C=C/C(=O)OCCCC(NCCCN1CCOCC1)C(=O)NCCCCCCCC/C=C\CCCCCCCC] | 661.58 | 661.80 |
| H45 (I-36) | C₄₀H₇₉N₃O₄ | 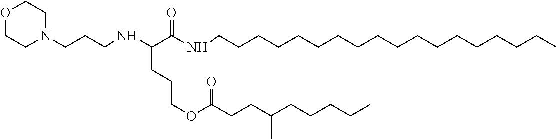 [SMILES: CCCCCCCCCCCCCCCCCCNC(=O)C(CCCOC(=O)CCC(C)CCCCC)NCCCN1CCOCC1] | 665.61 | 665.64 |
| I-37 | C₄₁H₈₁N₃O₃ | 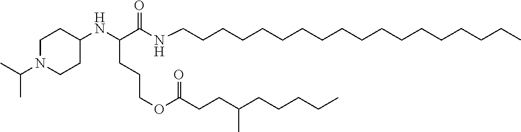 [SMILES: CCCCCCCCCCCCCCCCCCNC(=O)C(CCCOC(=O)CCC(C)CCCCC)NC1CCN(C(C)C)CC1] | 664.12 | |
| H1 (I-38) | C₃₂H₆₅N₃O | 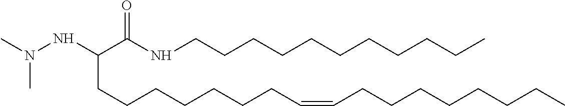 [SMILES: CCCCCCCC/C=C\CCCCCCCC(NN(C)C)C(=O)NCCCCCCCCCCC] | 508.51 | 508.52 |

### LNP Synthesis

LNPs were synthesized by mixing an aqueous phase containing the mRNA with an organic phase containing the lipids at a 3:1 volume ratio and a lipid/mRNA weight ratio of 10:1. LNPs were formulated by pipetting for all in vitro work without dialysis. LNPs for in vivo work were formulated using a T junction device via syringe pumps and buffer exchanged by dialysis against 1×PBS in a 10,000 MWCO cassette (Thermo Fisher) at 4° C. for 12 h before animal injection. The organic phase was prepared by dissolving a mixture of the synthesized ionizable lipid, helper phospholipid DSPC (Avanti) or DOPE (Avanti), cholesterol (Avanti), and C14-PEG2000 (Avanti) in ethanol at a predetermined molar ratio: 50:10:38.5:1.5 for DSPC formulation; 35:16:46.5:2.5 for DOPE formulation (35). SM-102 was purchased from Echelon Bioscience. The aqueous phase was prepared with mRNA in 10 mM citrate buffer (pH 4.0, Fisher). Three types of mRNA were included in the study: mLuc (firefly luciferase mRNA, TriLink), mCre (Cre-recombinase mRNA, 5-methoxyuridine (5moU), TriLink), and mOVA (full-length ovalbumin mRNA, 5moU, TriLink).

### Virtual Library

The Ugi combinatorial chemistry method was used to design diverse head groups, connecting groups, and two distinct alkyl chains. To be specific, the Markush Editor in the ChemAxon Marvin Suite (Marvin 23.4.0, ChemAxon) was used. The resulting virtual library contained approximately 60,000 lipid structures which were then exported into SMILES strings. This virtual library compromises multiple carbon chains, from C6 to C26. In addition, the presence or absence of ester bonds and their position in the carbon chain are used to improve the chemical diversity of the virtual library. The surface charge of LNP is usually determined by the lipids' head groups. In addition, the head group is important for mRNA binding. Amine groups are commonly used as lipids' head groups to form hydrogen bonds with mRNA, especially those containing tertiary amine.

### Experimental Library

The experimental library is described above and as noted, contained 20 head groups, 12 carbon chains with ester bonds, and 5 carbon chains with isocyanide head groups. 1200 lipids were selected for chemical synthesis and in vitro mTP experiments in HeLa and RAW 264.7 cell lines. The corresponding mTP was labelled in cells to each compound for the 1,200 lipids library. These data were generated by ChemAxon Marvin Suite into SMILE files (SMILE files in SI).

### Candidate Library

The final library used for model prediction was a filtered subset of the virtual library. The filtering contains three steps based on availability and rationality. First, the lipids containing tertiary amine structures were retained. Second, tail chains that were too long (>C18) or too short (<C10) based on expert knowledge of plausible ionizable lipid design were removed. Last, only those reagents commercially available for further validation of the model were selected. Upon completion of the filtering process, the final candidate library comprised approximately 12,000 lipids (SMILE files in SI), with the following 24 unique head groups:

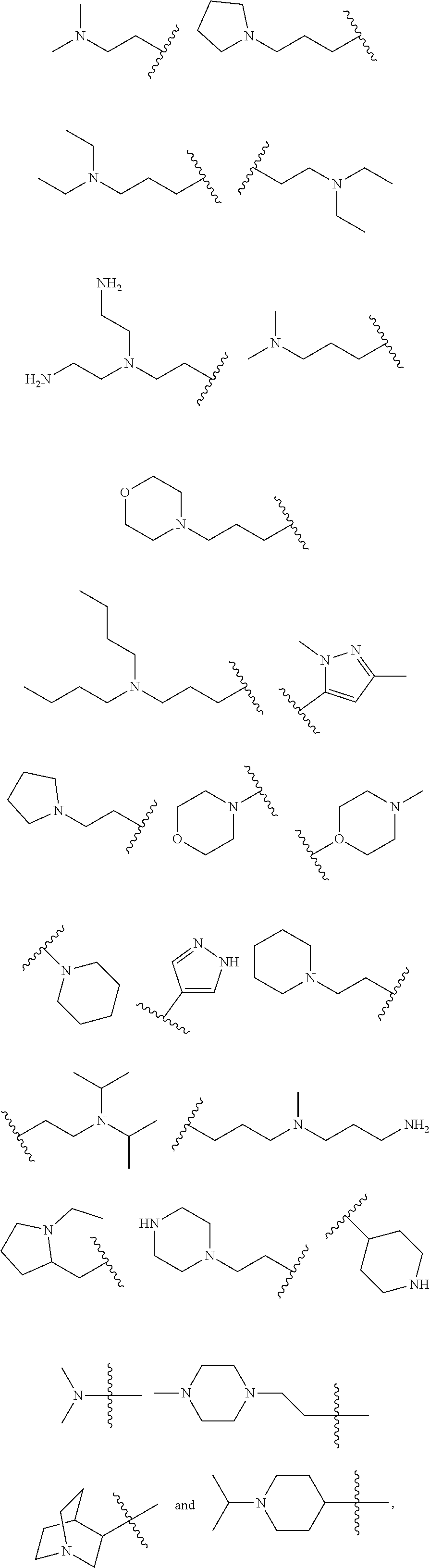

unique tail types of Tail B and Tail C in this arrangement (See for example, the tails noted above for the R² and R³ groups). In the prediction step of the platform, the model proposed the most promising lipids by predicting and ranking on the candidate library.

### Molecular Graph Construction

Molecular structures can be naturally represented as graphs where atoms are nodes and bonds are edges. For each molecule, the SMILES representation is converted into a molecular graph using RDKit, and later input to the neural network model in the platform. This representation captures the topological structure and properties of a molecule effectively. An ionizable lipid molecule graph G is defined as G=(V, E), where nodes V represent the atoms and edges E represent chemical bonds. The atom node features include the atom type (as on the periodic table) and a flag indicating whether the whole molecule it belongs to is chiral. For a node v, the features are constructed in a two-dimensional vector, hᵥ∈N². Edge features are constructed based on respective chemical bond types (i.e., single, double, triple, or aromatic bonds) and the stereochemical directionality (i.e., the rdchem.BondDir in RDKit. Similarly, the edge features form another two-dimensional vector for each bond between atom v and u, ϵ_{v,u}∈N².

### Materials and Methods for LNP Characterization and Screening

### In Vitro High-Throughput Screening

LNPs for in vitro screening were prepared in a 96-well plate by direct pipette mixing of the ethanol and the aqueous phase. LNPs/mLuc complex were added to 96-well plates pre-seeded with HeLa cells at the dosage level of 100 ng mLuc/10,000 cells. After overnight incubation, mLuc transfection efficiency was measured using the Bright-Glo Luciferase Assay System (Promega) according to the manufacturer's instructions. The relative luminescence unit (RLU) was quantified by the Cytation 1 Cell Imaging Multimode Reader (BioTek).

### Animals and Cells

HeLa and HepG2 cells were purchased from ATCC. THP-1 monocyte cell line (THP-1 Dual cells) was purchased from InvivoGen. B16-F10-Luc cells were kindly provided by Dr. Gang Zheng from Princess Margaret Cancer Center, University Health Network, Toronto. Cells were grown in RPMI 1640 media containing 10% fetal bovine serum (FBS) and Penicillin-Streptomycin (Gibco) according to manufacturer protocol in a humidified incubator containing 5% CO₂ at 37° C. Bone marrow dendritic cells (BMDCs) were prepared with the previously reported protocol (53). Briefly, bone marrow cells, flushed from the femurs of C57BL/6J mice, were cultured in the dendritic cell medium: RPMI 1640 supplemented with 10% FBS, Penicillin-Streptomycin (Gibco), and 20 ng ml⁻¹ GM-CSF (PeproTech). Then medium replacement was performed every 2 days. On day 7, immature dendritic cells that are nonadherent or loosely adhering were collected and seeded at 10,000 cells per well in a 96-well plate. All animal studies were approved and conducted in compliance with the University Health Network Animal Resources Centre guidelines. Female C57BL/6 and BALB/c mice (4-8 weeks) were obtained from the Jackson Laboratory. mTmG (Gt(ROSA)26Sor^{tm4(ACTB-tdTomato,-EGFP)Luo}/J) mice were kindly provided by Dr. Agostino Pierro from the Hospital for Sick Children, Toronto. For tumor model establishment, B16-F10-Luc cells (10⁶ cells/mouse) were implanted subcutaneously in the right flank of C57BL/6 mice (female, 6 weeks old). Tumor volumes were measured by IVIS Spectrum Imaging System (PerkinElmer). Luminescence was quantified using the Living Image software (PerkinElmer).

### Bioluminescence

At 6 h after the injection of the mRNA LNPs, mice were intraperitoneally (IP) injected with 0.2 ml D-luciferin (10 mg/ml in PBS). Mice were anesthetized in a ventilated anesthesia chamber with 1.5% isoflurane in oxygen and imaged 10 min after the injection using the IVIS Spectrum Imaging System (PerkinElmer). Luminescence was quantified using the Living Image software (PerkinElmer).

### LNP Formulation Optimization

Design of Experiment (DOE) methodology was used to optimize the six independent parameters known to influence the transfection and encapsulation efficiency of LNPs: (I) ratio of ionizable lipid to mRNA mass, (II) ionizable lipid molar composition, (Ill) phospholipid type, (IV) phospholipid molar composition, (V) PEGylated lipid molar composition, and (VI) aqueous-phase pH. DOE was carried out using JMP 13 software (SAS Institute). Results for LNP formulations with ionizable lipids H278 (I-A) and R080 are shown in the two tables below:

H278

| | Factor | | | | Response |
|---|---|---|---|---|---|
| | Lipid/mRNA | Ionizable | | | In Vitro |
| | weight | Lipid | DOPE | PEG | Hela |
| Nmuber | ratio | (mol %) | (mol %) | (mol %) | (Log₂) |
| 1 | 7.5 | 60 | 20 | 1.5 | 9.246 |
| 2 | 7.5 | 60 | 15 | 0.5 | 11.086 |
| 3 | 15 | 30 | 10 | 1.5 | 4.466 |
| 4 | 7.5 | 30 | 20 | 2.5 | 8.412 |
| 5 | 7.5 | 30 | 10 | 2.5 | 2.616 |
| 6 | 7.5 | 60 | 10 | 2.5 | −0.071 |
| 7 | 15 | 60 | 10 | 2.5 | 8.257 |
| *8 | 15 | 60 | 20 | 0.5 | 12.244 |
| 9 | 15 | 60 | 10 | 0.5 | 8.169 |
| 10 | 11.25 | 60 | 20 | 2.5 | 9.016 |
| 11 | 15 | 45 | 20 | 2.5 | 8.923 |
| 12 | 7.5 | 45 | 10 | 0.5 | 6.256 |
| 13 | 15 | 30 | 20 | 0.5 | 9.252 |
| 14 | 11.25 | 45 | 15 | 1.5 | 8.382 |
| 15 | 15 | 30 | 15 | 2.5 | 4.237 |
| 16 | 7.5 | 30 | 20 | 0.5 | 8.024 |
| 17 | 11.25 | 30 | 10 | 0.5 | 6.939 |

*Formulation 8 was the top performing for H278

R080

| | Factor | | | | Response |
|---|---|---|---|---|---|
| | Lipid/mRNA | Ionizable | | | In Vitro |
| | weight | Lipid | DOTAP | PEG | RAW 264.7 |
| Nmuber | ratio | (mol %) | (mol %) | (mol %) | (Log₂) |
| 1 | 7.5 | 60 | 20 | 1.5 | 6.518 |
| #2 | 7.5 | 60 | 15 | 0.5 | 6.680 |
| 3 | 15 | 30 | 10 | 1.5 | 4.327 |
| 4 | 7.5 | 30 | 20 | 2.5 | 4.414 |
| 5 | 7.5 | 30 | 10 | 2.5 | 2.705 |
| 6 | 7.5 | 60 | 10 | 2.5 | 2.825 |
| 7 | 15 | 60 | 10 | 2.5 | 3.102 |
| 8 | 15 | 60 | 20 | 0.5 | 5.172 |
| 9 | 15 | 60 | 10 | 0.5 | 5.574 |
| 10 | 11.25 | 60 | 20 | 2.5 | 5.115 |
| 11 | 15 | 45 | 20 | 2.5 | 4.621 |
| 12 | 7.5 | 45 | 10 | 0.5 | 6.123 |
| 13 | 15 | 30 | 20 | 0.5 | 5.604 |
| 14 | 11.25 | 45 | 15 | 1.5 | 3.333 |
| 15 | 15 | 30 | 15 | 2.5 | 3.628 |
| 16 | 7.5 | 30 | 20 | 0.5 | 6.443 |
| 17 | 11.25 | 30 | 10 | 0.5 | 5.150 |

#Formulation 2 was the top performing for R080

### LNP Characterization

The size, polydispersity index and zeta potentials of LNPs were measured using dynamic light scattering (ZetaPALS, Brookhaven Instruments). Diameters are reported as the intensity mean peak average. To calculate the nucleic acid encapsulation efficiency, a modified Quant-iT RiboGreen RNA assay (Invitrogen) was used as previously described (35).

### TNS Binding Assay for pKa

TNS binding assay was used to determine the LNP apparent pKa. LNPs were diluted and mixed with TNS in a total volume of 150 ul of buffered solution with the final concentrations: (LNP, 75 μM) and (TNS, 6 μM). The buffered solutions with pH ranging from 3 to 10 were made containing 20 mM boric acid, 10 mM imidazole, 10 mM sodium acetate, 10 mM glycylglycine, and 25 mM NaCl. The Cytation 5 Cell Imaging Multi-Mode Reader (Biotek) was used to read the fluorescence (Ex321/Em445). The pH was measured in each well after TNS addition. Mathematica (Wolfram Research) was used to fit the fluorescence data to the Henderson-Hasselbalch equation to provide the pKa.

### Immunohistochemistry

Gt(ROSA)26Sor^{tm4(ACTB-tdTomato,-EGFP)Luo}/j mTmG mice were intramuscularly injected with LNPs/mCre containing 10 μg mRNA formulated with iso-A111B5 or SM-102. Thirty-six hours after the injection, the mice were sacrificed for tissue collection: muscle (quadricep), liver, and spleen. All collected tissues were fixed in 4% buffered paraformaldehyde overnight at 4° C., then dehydrated in 30% sucrose overnight at 4° C. Next, cryosection was performed on tissues after being frozen in OCT. Tissue sections were mounted with DAPI (1:1000). For the tumor model, mice were sacrificed for tumor collection. For tumor sections, slides were stained with DAPI (1:1000), anti-CD8 (APC, BioLegend, 1:200), and anti-IFN-γ (AF488, BioLegend, 1:200). Zeiss LSM900 was used to obtain and process the images.

### Enzyme-Linked Immunosorbent Assay (ELISA) for Antibody Titer

Indirect ELISA was performed to measure the antibody titer. The high-binding 96-well plates (Corning) were covered with 100 μL of ovalbumin protein (InvivoGen) with a concentration of 20 μg/ml at 4° C. overnight. The plates were then washed four times with PBS-T and blocked by 1% bovine serum albumin solution (Sigma-Aldrich). The sera collected from immunized and control mice were initially diluted at 1:100. Then, a two-fold serial dilution was performed on all serum samples. 100 μL of samples were added to the plates for 1 h at 37° C. Then the plates were washed and then incubated with goat anti-mouse IgG (H+L) secondary antibody with HRP at 1:10,000 (InvitroGen) for 1 h at 37° C. Next, the plates were washed and 50 μL of TMB ELISA substrate solution was added for 15 min incubation at room temperature. Then 50 μL of stop solution (0.16M sulfuric acid) was added. Cytation 1 Cell Imaging Multimode Reader (BioTek) was used to measure the Optical Density (OD₄₅₀).

### Flow Cytometry

Antibodies purchased from BioLegend and Thermo Scientific for flow cytometry are listed in Supplementary Table 3. For the flow cytometry analysis of surface markers, cells from mouse BMDCs, tumors, splenocytes, and lymph nodes were pre-incubated with anti-CD16/32 antibody (BioLegend) and stained on ice with fluorophore-conjugated antibody. For the staining of intracellular markers (IFN-γ), cells were fixed and permeabilized using the Foxp3/Transcription Factor Staining Buffer Set (eBioscience). Then, cells were stained with anti-IFN-γ. Flow data were acquired on a CytoFlex S flow cytometer and analyzed using FlowJo software.

### Splenocyte Stimulation and Cytokine Study

BALB/c mice were immunized with full-length OVA mRNA (5moU, TriLink) encapsulated in either SM-102 LNP or iso-A11B5C1 LNP (n=4). Both vaccinated groups, as well as the unvaccinated group (PBS injection) (n=4), were sacrificed at D28 for spleen collection. Splenocytes were then disassociated as previously described (54). In brief, spleens were homogenized and passed through the 70 μm cell strainer (Fisherbrand). The cells were washed twice with PBS, followed by RBC cell lysis treatment. Next, splenocytes were plated at a density of 10 million cells per milliliter in 48-well culture plates with culture medium RPMI 1640 supplemented with 10% FBS, Penicillin-Streptomycin (Gibco). Then splenocytes were stimulated with either one of the OVA-derived peptides: H-2K^{b}-restricted MHC class I epitope (InvivoGen) or I-Ad-restricted OVA MHC class II epitope (InvivoGen) at the concentration of 10 μg/ml. Supernatants of splenocytes were collected for cytokine study after 72 h. The supernatants were diluted 4 times for 20 different cytokines measurements using Quantibody® Mouse Cytokine Array 1 Kit (RayBiotech, USA) according to the manufacturer's protocol. The assay was analyzed using GenePix® Professional 4200 Microarray Scanner at 530 nm.

### Statistics

GraphPad Prism version 9.4.1 (GraphPad Software) was used for all statistical analysis and for generating the plots. Data are reported as mean±SD. Significant differences between groups were analyzed by using Student's t-test or Tukey's multiple comparisons test based on test groups. *=p-value<0.05, **=p-value<0.01, ***=p-value<0.005, ****=p-value<0.001.

### Materials and Methods for the AGILE Platform Architecture

In one embodiment, the AGILE platform, such as system or platform 100 of FIG. 1, comprises three major components: (1) The embedding layers to project node and edge features into learnable vectors, (2) the graph encoder for modeling molecular structures, and (3) the descriptor encoder for modeling molecular properties.

The embeddings layers project the integer features in hᵥ and ϵ_{v,u} to learnable feature vectors

$${h}_{v}^{( 0 )} and {ϵ}_{v , u}^{( 0 )} ,$$

which can be optimized later during the training of the whole neural network. Here, both

$${h}_{v}^{( 0 )} and {ϵ}_{v , u}^{( 0 )}$$

are R^{d} vectors, and d is a predefined size of embedding dimensions. The embedding vectors were first obtained for both atom type and charity features in h_{v,u} and the two vectors were added elementwise to output the

$${h}_{v}^{( 0 )} :$$

$${h}_{v}^{( 0 )} = {Emb}_{h , 0}^{( 0 )} ( {h}_{v} [ 0 ] ) + {Emb}_{h , 1}^{( 0 )} ( {h}_{v} [ 1 ] ) , \qquad Eq . 3$$

here [i] denotes the i-th element in the vector. Emb is the embedding layer projection. In this work, PyTorch Embedding layers were used. Similarly, the

$${ϵ}_{v , u}^{( 0 )}$$

is computed as:

$${ϵ}_{v , u}^{( 0 )} = {Emb}_{ϵ , 0}^{( 0 )} ( {ϵ}_{v , u} [ 0 ] ) + {Emb}_{ϵ , 1}^{( 0 )} ( {ϵ}_{v , u} [ 1 ] ) . \qquad Eq . 4$$

With respect to the Graph encoder, Graph Isomorphism Network (GIN)⁶⁴, a type of graph neural network (GNN), was used to operate on the input molecule graphs and to learn a representation vector for each ionizable lipid molecule. GIN can directly propagate messages among nodes and edges on a graph structure and thus is suitable for processing molecular graphs. Additionally, the advantage of GIN over other GNNs is its ability to distinguish between different graph structures, including isomorphic graphs. This makes GIN more expressive than many other GNNs and a suitable tool for tasks involving molecular graph data. The implemented GIN model follows similar structures used in MolCLR, which underwent pre-training on a repertoire of over million small molecules, so as to benefit from the general pre-trained molecular model of MolCLR as a warm start for the platform. The update rule of GIN for a node representation on the k^{th} layer is given as:

$${h}_{v}^{( k )} = M L {P}^{( k )} ( ( 1 + {ε}^{( k )} ) . \cdot {h}_{v}^{( k - 1 )} + {\sum }_{u ∈ N ( v )} {m}_{u}^{( k - 1 )} ) , \qquad Eq . 5$$

where

$${h}_{v}^{( k )}$$

is the representation of node v at the k^{th} layer and N(v) denotes the set of neighbors of node v, and ε is a learnable parameter. MLP denotes the stacked fully connected neural network layers. The

$${m}_{u}^{( k - 1 )}$$

is the message propagated between a neighbor u to the current node. It is computed as the sum of node and edge contributions:

$${m}_{u}^{( k - 1 )} = {h}_{u}^{( k - 1 )} + {ϵ}_{v , u}^{( k - 1 )} , \qquad Eq . 6$$

$${ϵ}_{v , u}^{( k - 1 )} = {Emb}_{ϵ , 0}^{( k - 1 )} ( {ϵ}_{v , u} [ 0 ] ) + {Emb}_{ϵ , 1}^{( k - 1 )} ( {ϵ}_{v , u} [ 1 ] ) .$$

Notably,

$${h}_{v}^{( 0 )} and {ϵ}_{v , u}^{( 0 )}$$

from Eq. and Eq. are used for the first GIN layer.

A total of K GIN layers was stacked for the entire Graph Encoder. To extract the feature of the whole molecular graph h_{G}, a mean pooling operation was implemented on the final layer to integrate all the node features:

$${h}_{G} = Mean ( \{ {h}_{v}^{( K )} : v ∈ G \} ) . \qquad Eq . 7$$

Another fully connected layer was used to transform h_{G} to the final lipid representation z_{G}.

$${z}_{G} = MLP ( {h}_{G} ) . \qquad Eq . 8$$

In addition to the structure features encoded by the GIN, the platform utilized another descriptor encoder to explicitly model molecular properties. In one embodiment, the molecular descriptor encoder contributed a more stabilized training optimization. This benefit may come from the straight-forward utilization of computed properties during the optimization, which relieves the model from learning all information from the structure alone. In the implementation of the platform, the molecular descriptors derived from Mordred (32) calculations were used, which contained over 1,000 common descriptors for each molecule, including the num of atoms, num of bonds, etc. These features were encoded by gully connected layers into a representation for these properties, zₚ∈R^{d}^{ₚ}:

$${z}_{p} = MLP ( descripors ) . \qquad Eq . 9$$

The final representation of the molecule was the concatenation of the structure and property representations:

$$z = [ {z}_{G} , {z}_{p} ] , \qquad Eq . 10$$

where [,] denotes the concatenation of two vectors.

In one example, the Graph Encoder in the model consisted of a five-layer GIN with ReLU activation. To extract a 512-dimensional LNP representation, an average pooling layer was applied to each lipid molecular graph. A single hidden layer MLP was then employed to map the representation into a 256-dimensional latent space. During model pre-training, the contrastive loss was optimized using the Adam optimizer⁶⁵, with a weight decay of 10⁻⁵, and the temperature was set to 0.1. The pre-training process involved a batch size of 512 for 100 epochs.

For model fine-tuning, an additional MLP with one hidden layer was introduced to map the molecular descriptors into 100-dimensional latent vectors. These vectors were concatenated with the 256-dimensional LNP representation obtained from the GNN encoder. Subsequently, a two-layer MLP was utilized to derive the final prediction value from the concatenated vector. The fine-tuning process employed the Adam optimizer with a weight decay of 10⁻⁶ to optimize the loss (Error! Reference source not found). Each fine-tuned model was trained using a batch size of 128 for 30 epochs.

### Top 1-15 Lipid Candidate Structures Identified for Hela Cells by the AGILE Model:

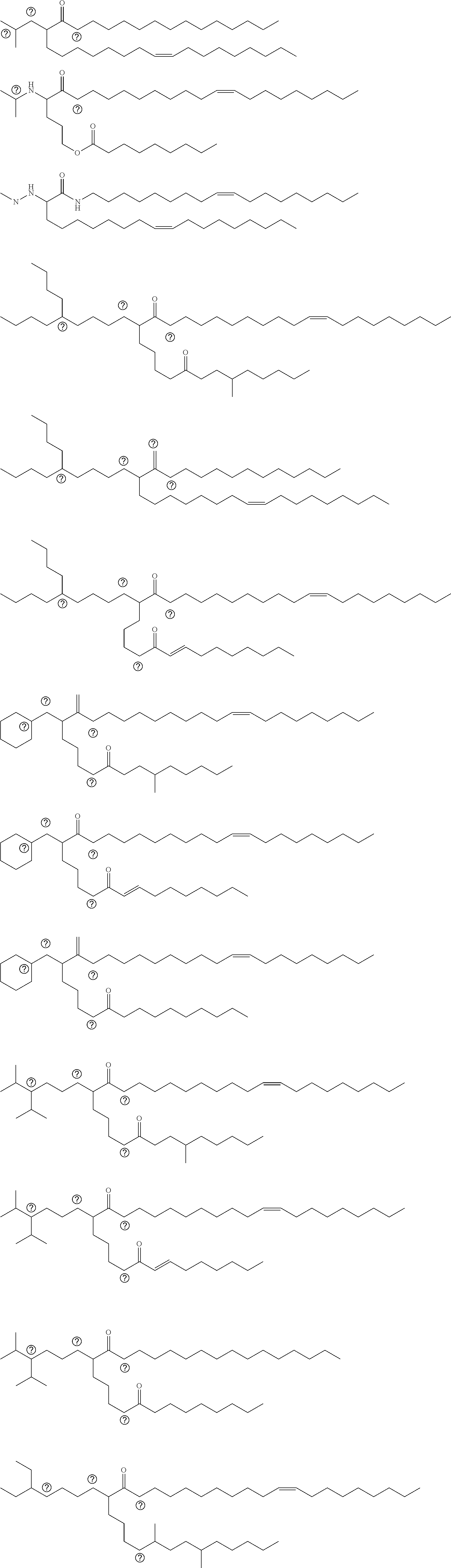

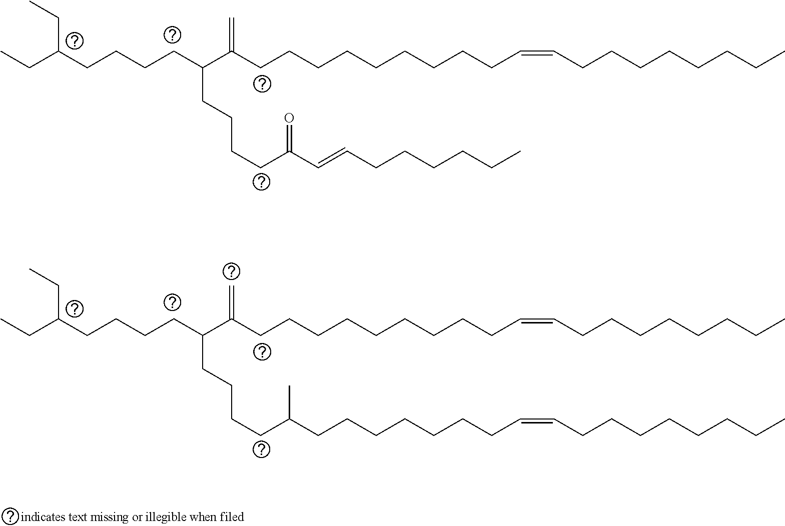

### Top 15 Predicted Lipids for Raw 264.7:

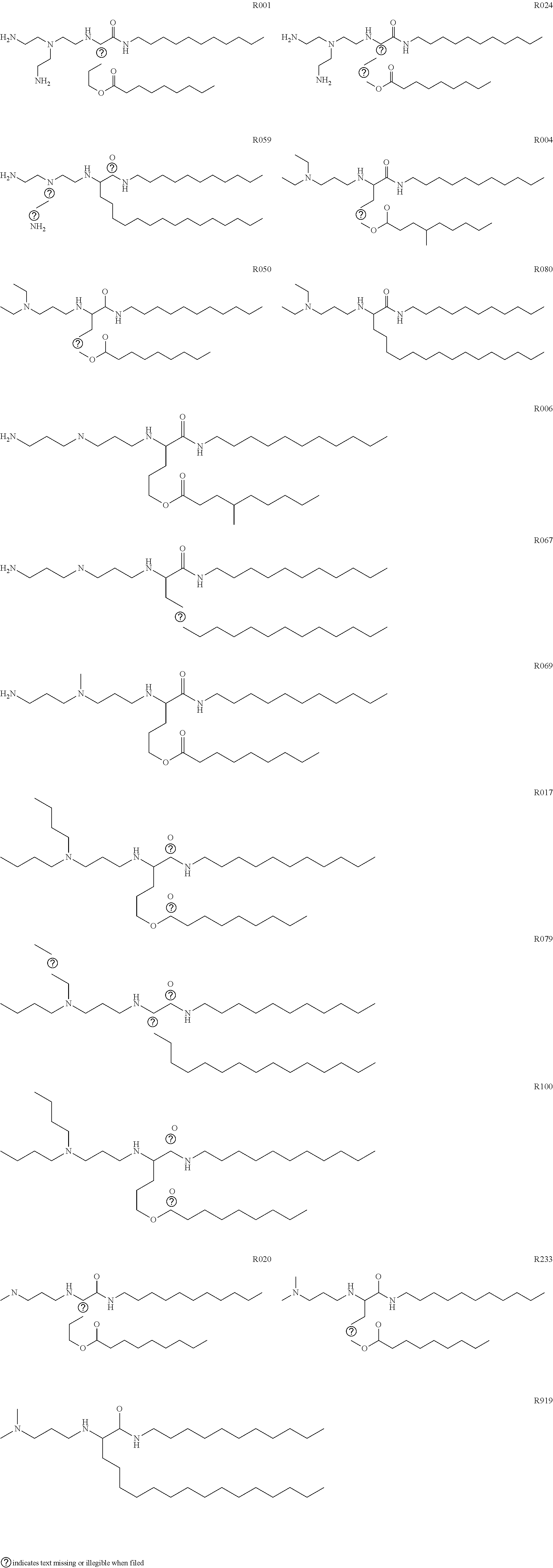

### FULL CITATIONS FOR DOCUMENTS REFERENCED IN THE APPLICATION

1. A. J. Barbier, A. Y. Jiang, P. Zhang, R. Wooster, D. G. Anderson, The clinical progress of mRNA vaccines and immunotherapies. Nature Biotechnology 40, 840-854 (2022).
2. N. Pardi, M. J. Hogan, D. Weissman, Recent advances in mRNA vaccine technology. Current Opinion in Immunology 65, 14-20 (2020).
3. X. Hou, T. Zaks, R. Langer, Y. Dong, Lipid nanoparticles for mRNA delivery. Nature Reviews Materials 6, 1078-1094 (2021).
4. J. C. Kaczmarek, P. S. Kowalski, D. G. Anderson, Advances in the delivery of RNA therapeutics: from concept to clinical reality. Genome Medicine 9, 60 (2017).
5. M. A. Maier et al., Biodegradable lipids enabling rapidly eliminated lipid nanoparticles for systemic delivery of RNAi therapeutics. Mol Ther 21, 1570-1578 (2013).
6. S. Sabnis et al., A Novel Amino Lipid Series for mRNA Delivery: Improved Endosomal Escape and Sustained Pharmacology and Safety in Non-human Primates. Mol Ther 26, 1509-1519 (2018).
7. A. Akinc et al., The Onpattro story and the clinical translation of nanomedicines containing nucleic acid-based drugs. Nature Nanotechnology 14, 1084-1087 (2019).
8. L. A. Jackson et al., An mRNA Vaccine against SARS-CoV-2—Preliminary Report. New England Journal of Medicine 383, 1920-1931 (2020).
9. F. P. Polack et al., Safety and Efficacy of the BNT162b2 mRNA Covid-19 Vaccine. New England Journal of Medicine 383, 2603-2615 (2020).
10. D. F. Li et al., Nanomaterials for <scp>mRNA</scp>-based Therapeutics: Challenges and Opportunities. Bioengineering &amp; Translational Medicine 10.1002/btm2.10492 (2023).
11. J. Seitzer, NTLA-2002: CRISPR/Cas9-mediated gene knockout of KLKB1 to treat hereditary angioedema. Journal of Allergy and Clinical Immunology 147, AB147 (2021).
12. E. M. Agency (2021) Assessment report on Comirnaty (Common Name: COVID-19 mRNA Vaccine)(Nucleoside-Modified) Procedure No. EMEA/H/C/005735/0000. (European Medicines Agency Amsterdam, The Netherlands). 13. H. Tanaka et al., Delivery of Oligonucleotides Using a Self-Degradable Lipid-Like Material. Pharmaceutics 13, 544 (2021).
14. H. Zhang et al., Rational design of anti-inflammatory lipid nanoparticles for mRNA delivery. Journal of Biomedical Materials Research Part A 110, 1101-1108 (2022).
15. K. J. Hassett et al., Optimization of Lipid Nanoparticles for Intramuscular Administration of mRNA Vaccines. Molecular Therapy—Nucleic Acids 15, 1-11 (2019).
16. X. Han et al., An ionizable lipid toolbox for RNA delivery. Nature Communications 12, 7233 (2021).
17. X. Han et al., An ionizable lipid toolbox for RNA delivery. Nature Communications 12 (2021).
18. K. A. Hajj, K. A. Whitehead, Tools for translation: non-viral materials for therapeutic mRNA delivery. Nature Reviews Materials 2, 1-17 (2017).
19. P. S. Kowalski, A. Rudra, L. Miao, D. G. Anderson, Delivering the messenger: advances in technologies for therapeutic mRNA delivery. Molecular Therapy 27, 710-728 (2019).
20. C. Meng, Z. Chen, G. Li, T. Welte, H. Shen, Nanoplatforms for mRNA therapeutics. Advanced Therapeutics 4, 2000099 (2021).
21. L. Miao et al., Delivery of mRNA vaccines with heterocyclic lipids increases anti-tumor efficacy by STING-mediated immune cell activation. Nat Biotechnol 37, 1174-1185 (2019).
22. C. Hulme, V. Gore, “Multi-component reactions: emerging chemistry in drug discovery” ‘from xylocain to crixivan’. Curr Med Chem 10, 51-80 (2003).
23. L. Miao et al., Delivery of mRNA vaccines with heterocyclic lipids increases anti-tumor efficacy by STING-mediated immune cell activation. Nature Biotechnology 37, 1174-1185 (2019).
24. H. Lv, S. Zhang, B. Wang, S. Cui, J. Yan, Toxicity of cationic lipids and cationic polymers in gene delivery. Journal of controlled release 114, 100-109 (2006).
25. M. C. Filion, N. C. Phillips, Toxicity and immunomodulatory activity of liposomal vectors formulated with cationic lipids toward immune effector cells. Biochimica et Biophysica Acta (BBA)-Biomembranes 1329, 345-356 (1997).
26. M. Sedic et al., Safety evaluation of lipid nanoparticle-formulated modified mRNA in the sprague-dawley rat and cynomolgus monkey. Veterinary pathology 55, 341-354 (2018).
27. B. Li et al., Combinatorial design of nanoparticles for pulmonary mRNA delivery and genome editing. Nature Biotechnology, 1-6 (2023).
28. S. Ali, J. M. Smaby, M. M. Momsen, H. L. Brockman, R. E. Brown, Acyl chain-length asymmetry alters the interfacial elastic interactions of phosphatidylcholines. Biophys J 74, 338-348 (1998).
29. D. Yuan, Z. Duan, Y. Rao, M.-W. Ding, New efficient synthesis of isoquinoline-1,3(2H,4H)-diones and isoindolin-1-ones via sequential Ugi/cyclization reaction. Tetrahedron 72, 338-346 (2016).
30. Z.-L. Ren, P. He, W.-T. Lu, M. Sun, M.-W. Ding, Synthesis of iminoisoindolinones via a cascade of the three-component Ugi reaction, palladium catalyzed isocyanide insertion, hydroxylation and an unexpected rearrangement reaction. Organic & Biomolecular Chemistry 16, 6322-6331 (2018).
31. M. H. R. Carvalho et al., Solvent Dependent Competitive Mechanisms for the Ugi Multicomponent Reaction: A Joint Theoretical and Experimental Study in the α-Acyl Aminocarboxamides vs α-Amino Amidines Formation. The Journal of Organic Chemistry 87, 11007-11020 (2022).
32. J. Zhang, S.-X. Lin, D.-J. Cheng, X.-Y. Liu, B. Tan, Phosphoric Acid-Catalyzed Asymmetric Classic Passerini Reaction. Journal of the American Chemical Society 137, 14039-14042 (2015).
33. A. Kumar, D. Saxena, M. K. Gupta, Boric acid catalyzed Ugi three-component reaction in aqueous media. RSC Advances 3, 4610-4612 (2013).
34. Y. Zhang, C. Sun, C. Wang, K. E. Jankovic, Y. Dong, Lipids and Lipid Derivatives for RNA Delivery. Chemical Reviews 121, 12181-12277 (2021).
35. K. J. Kauffman et al., Optimization of lipid nanoparticle formulations for mRNA delivery in vivo with fractional factorial and definitive screening designs. Nano letters 15, 7300-7306 (2015).
36. K. A. Whitehead et al., Degradable lipid nanoparticles with predictable in vivo siRNA delivery activity. Nature Communications 5, 4277 (2014).
37. Y. Li et al., Protein and mRNA Delivery Enabled by Cholesteryl-Based Biodegradable Lipidoid Nanoparticles. Angewandte Chemie 132, 15067-15074 (2020).
38. K. A. Hajj et al., Branched-Tail Lipid Nanoparticles Potently Deliver mRNA In Vivo due to Enhanced Ionization at Endosomal pH. Small 15, 1805097 (2019).
39. C. D. Sago et al., High-throughput in vivo screen of functional mRNA delivery identifies nanoparticles for endothelial cell gene editing. Proceedings of the National Academy of Sciences 115, E9944-E9952 (2018).
40. S. A. Dilliard, Q. Cheng, D. J. Siegwart, On the mechanism of tissue-specific mRNA delivery by selective organ targeting nanoparticles. Proceedings of the National Academy of Sciences 118, e2109256118 (2021).
41. M. J. Carrasco et al., Ionization and structural properties of mRNA lipid nanoparticles influence expression in intramuscular and intravascular administration. Communications Biology 4 (2021).
42. M. D. Muzumdar, B. Tasic, K. Miyamichi, L. Li, L. Luo, A global double-fluorescent Cre reporter mouse. genesis 45, 593-605 (2007).
43. D. L. Sellers et al., Targeted axonal import (TAxI) peptide delivers functional proteins into spinal cord motor neurons after peripheral administration. Proceedings of the National Academy of Sciences 113, 2514-2519 (2016).
44. J. S. Tregoning et al., Formulation, inflammation, and RNA sensing impact the immunogenicity of self-amplifying RNA vaccines. Molecular Therapy—Nucleic Acids 31, 29-42 (2023).
45. M. B. Bloom et al., Identification of tyrosinase-related protein 2 as a tumor rejection antigen for the B16 melanoma. The Journal of experimental medicine 185, 453-460 (1997).
46. X. Han et al., Ligand-tethered lipid nanoparticles for targeted RNA delivery to treat liver fibrosis. Nature Communications 14, 75 (2023).
47. M. J. Carrasco et al., Ionization and structural properties of mRNA lipid nanoparticles influence expression in intramuscular and intravascular administration. Communications Biology 4, 956 (2021).
48. E. Kenjo et al., Low immunogenicity of LNP allows repeated administrations of CRISPR-Cas9 mRNA into skeletal muscle in mice. Nature Communications 12, 7101 (2021).
49. G. Tilstra et al., Iterative Design of Ionizable Lipids for Intramuscular mRNA Delivery. Journal of the American Chemical Society 145, 2294-2304 (2023).
50. N. Chaudhary, D. Weissman, K. A. Whitehead, mRNA vaccines for infectious diseases: principles, delivery and clinical translation. Nature Reviews Drug Discovery 20, 817-838 (2021).
51. A. Heine, S. Juranek, P. Brossart, Clinical and immunological effects of mRNA vaccines in malignant diseases. Molecular Cancer 20 (2021).
52. Y. J. Kwon, S. M. Standley, A. P. Goodwin, E. R. Gillies, J. M. J. Frechet, Directed Antigen Presentation Using Polymeric Microparticulate Carriers Degradable at Lysosomal pH for Controlled Immune Responses. Molecular Pharmaceutics 2, 83-91 (2005).
53. M. Sauter et al., Protocol to isolate and analyze mouse bone marrow derived dendritic cells (BMDC). STAR Protocols 3, 101664 (2022).
54. W. Wang, X. Ai, Primary culture of immature, naïve mouse CD4(+) T cells. STAR Protoc 2, 100756 (2021).

1. An AI-based method for ionizable lipid engineering for lipid nanoparticle (LNP)-based cargo molecule delivery, the method comprising:
designing a plurality of ionizable lipid structures based on a combination of a plurality of lipid headgroups, a plurality of first tailgroups and a plurality of second tailgroups;
training a machine learning model based on the plurality of ionizable lipid structures;
subsequently, selecting a first subset of ionizable lipid structures from the plurality of ionizable lipid structures and designating the first subset of ionizable lipid structures as experimental ionizable lipid structures;
synthesizing, using a high throughput synthetic method, each of the experimental ionizable lipid structures;
preparing a first set of separate lipid nanoparticles (LNPs), each LNP comprising a unique experimental ionizable lipid structure and the cargo molecule;
determining a corresponding cargo molecule transfection efficiency value of each of the prepared LNPs in a target cell;
further training the machine learning model based on the prepared LNPs and the corresponding cargo molecule transfection efficiency values; and
deploying the further trained machine learning model to identify at least one lead ionizable lipid structure.

2. The method of claim 1, wherein deploying the further trained machine learning model comprises:
selecting a second subset of ionizable lipid structures from the plurality of ionizable lipid structures and designating the second subset of ionizable lipid structures as ionizable lipid candidate structures;
predicting the cargo molecule transfection efficiency values of each of the ionizable lipid candidate structures based on the further trained machine learning model;
ranking the ionizable lipid candidate structures based on the corresponding predicted cargo molecule transfection efficiency values in accordance with a ranking scheme; and
identifying the at least one lead ionizable lipid structure based on the ranking of the ionizable lipid candidate structures for in-vitro and/or in-vivo validation.

3. The method of claim 2, further comprising:
ranking the ionizable lipid candidate structures by first ranking the corresponding cargo molecule transfection efficiency values based on identity of lipid headgroups; and
subsequently, ranking the cargo molecule transfection efficiency values within each headgroup based on tailgroups.

4. The method of claim 1, wherein synthesizing the experimental ionizable lipid structures comprises:
utilizing a one pot method for synthesizing an ionizable lipid of Formula I: 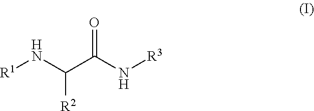
comprising reacting a compound of Formula A, a compound of Formula B and a compound of Formula C: 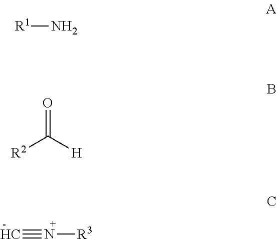
in the presence of a catalyst under conditions to provide the compound of Formula I,
wherein:
R¹ is a cyclic or acyclic group comprising at least one ionizable nitrogen atom;
R² is selected from C₁₋₃₀alkyl and C₂₋₃₀alkenyl, each of which is optionally interrupted by a hydrolysable group; and
R³ is selected from C₁₋₃₀alkyl and C₂₋₃₀alkenyl, each of which is optionally interrupted by a hydrolyzable group.

5. The method of claim 4, wherein the method of preparing the ionizable lipid of Formula I is performed using combinatorial chemistry procedures.

6. The method of claim 2, further comprising:
generating the ionizable lipid candidate structures from the plurality of ionizable lipid structures by identifying ionizable lipid structures comprising cationic lipids.

7. The method of claim 2, further comprising:
generating the ionizable lipid candidate structures from the plurality of ionizable lipid structures by identifying ionizable lipid structures comprising ionizable lipids with alkyl chains that are either below a first length threshold or above a second length threshold.

8. The method of claim 2, further comprising:
generating the ionizable lipid candidate structures from the plurality of ionizable lipid structures by identifying ionizable lipid structures comprising a predetermined group of reagents.

9. The method of claim 1, wherein the cargo molecule comprises siRNA, tRNA, circRNA or mRNA, or a combination thereof.

10. The method of claim 1, wherein the target cell is selected from: Hela cells, T-cells, stem cells, lung epithelial cells, pancreatic beta cells, natural killer cells, macrophages, dendritic cells, endothelial cells, chondrocytes, skeletal muscle cells, cardiomyocytes, neurons, retinal cells and hepatocytes.

11. An AI-based system for engineering ionizable lipids for lipid nanoparticle (LNP)-based cargo molecule delivery, the system comprising:
a processor,
a machine learning model coupled to the processor, and
a memory coupled to the processor and the machine learning model, the memory comprising a database;
the processor being configured to:
design a plurality of ionizable lipid structures based on combinatorial of a plurality of lipid headgroups, a plurality of first tailgroup and a plurality of second tailgroup;
store the plurality of ionizable lipid structures in the database in the memory;
train the machine learning model based on the plurality of ionizable lipid structures;
subsequently, select a first subset of ionizable lipid structures from the plurality of ionizable lipid structures and designate the first subset of ionizable lipid structures as experimental ionizable lipid structures;
store the first subset of ionizable lipid structures in the database of the memory;
receive a plurality of cargo molecule transfection efficiency values for a corresponding plurality of prepared lipid nanoparticles (LNPs), each LNP comprising a unique synthesized experimental ionizable lipid structure and the cargo molecule, wherein each experimental ionizable lipid structure is synthesized using a high throughput synthetic method and the cargo molecule transfection efficiency values are determined by testing the corresponding prepared lipid nanoparticle in a target cell;
store the received plurality of cargo molecule transfection efficiency values in the database of the memory;
further train the machine learning model based on the prepared LNPs and the corresponding cargo molecule transfection efficiency values; and
deploy the further trained machine learning model to identify at least one lead ionizable lipid structure.

12. The system of claim 11, wherein the processor is further configured to:
select a second subset of ionizable lipid structures from the plurality of ionizable lipid structures and designate the second subset of ionizable lipid structures as ionizable lipid candidate structures;
predict the cargo molecule transfection efficiency values of each of the ionizable lipid candidate structures based on the further trained machine learning model;
rank the ionizable lipid candidate structures based on the corresponding predicted cargo molecule transfection efficiency values in accordance with a ranking schema; and
identify the at least one lead ionizable lipid structure based on the ranking of the ionizable lipid candidate structures for in-vitro and/or in-vivo validation.

13. The system of claim 12, wherein the processor is further configured to:
rank the ionizable lipid candidate structures by first ranking the corresponding cargo molecule transfection efficiency values based on identity of lipid headgroups; and
subsequently, rank the cargo molecule transfection efficiency values within each headgroup based on tailgroups.

14. The system of claim 12, wherein the processor is further configured to:
generate the ionizable lipid candidate structures from the plurality of ionizable lipid structures by identifying ionizable lipid structures comprising cationic lipids.

15. The system of claim 12, wherein the processor is further configured to:
generate the ionizable lipid candidate structures from the plurality of ionizable lipid structures by identifying ionizable lipid structures comprising ionizable lipids with alkyl chains that are either below a first length threshold or above a second length threshold.

16. The system of claim 12, wherein the processor is further configured to:
generate the ionizable lipid candidate structures from the plurality of ionizable lipid structures by identifying ionizable lipid structures comprising a predetermined group of reagents.

17. The system of claim 11, wherein the machine learning model is selected from: Graph Neural Network (GNN), Convolutional Neural Network (CNN). Recurrent Neural Network (RNN), Transformer Model, Autoencoders, Capsule Network, Message Passing Neural Network (MPNN), Spectral CNN and Diffusion Neural Network.

18. The system of claim 11, wherein the processor is configured to train the machine learning model based on supervised, self-supervised, semi-supervised, unsupervised and/or reinforcement training.
